SEQUENCE LISTING (88 sequences)

SEQ ID NO: 1 - protein, 364 aa, Homo sapiens
MPLLLLLPLLWAGALAMDPNFWLQVQESVTVQEGLCVLVPCTFFHPIPYYDKNSPVHGYWFREGAIISRDSPVATNKLDQEVQEETQGRFRLLGDPSRNNCSLSIVDARRRDNGSYFFRMERGSTKYSYKSPQLSVHVTDLTHRPKILIPGTLEPGHSKNLTCSVSWACEQGTPPIFSWLSAAPTSLGPRTTHSSVLIITPRPQDHGTNLTCQVKFAGAGVTTERTIQLNVTYVPQNPTTGIFPGDGSGKQETRAGVVHGAIGGAGVTALLALCLCLIFFIVKTHRRKAARTAVGRNDTHPTTGSASPKHQKKSKLHGPTETSSCSGAAPTVEMDEELHYASLNFHGMNPSKDTSTEYSEVRTQ

SEQ ID NO: 2 - protein, 116 aa, synthetic construct [sequence omitted]

SEQ ID NO: 3 - protein, 111 aa, synthetic construct [sequence omitted]

SEQ ID NO: 4 - protein, 5 aa, synthetic construct
DSNIH

SEQ ID NO: 5 - protein, 17 aa, synthetic construct
YIYPYNGGTDYNQKFKN

SEQ ID NO: 6 - protein, 7 aa, synthetic construct
GNPWLAY

SEQ ID NO: 7 - protein, 15 aa, synthetic construct
RASESLDNYGIRFLT

SEQ ID NO: 8 - protein, 7 aa, synthetic construct
AASNQGS

SEQ ID NO: 9 - protein, 9 aa, synthetic construct
QQTKEVPWS

SEQ ID NO: 10 - protein, 117 aa, synthetic construct
QVQLVQSGAEVKKPGSSVKVSCKASGGTFSDYAISWVRQAPGQGLEWMGRIIPILGVADYAQKFQGRVTITADKSTRTAYMELSSLRSEDTAVYYCARNWADAFDIWGQGTMVTVSS

SEQ ID NO: 11 - protein, 106 aa, synthetic construct
DIQLTQSPSSLSASVGDRVTITCRASQGISSVLAWYQQKPGKAPKLLIYDASSLESGVPSRFSGSGSGTDFTLTISSLQPEDFATYYCQQFDSSITFGQGTKLEIK

SEQ ID NO: 12 - protein, 5 aa, synthetic construct
DYAIS

SEQ ID NO: 13 - protein, 17 aa, synthetic construct
RIIPILGVADYAQKFQG

SEQ ID NO: 14 - protein, 8 aa, synthetic construct
NWADAFDI

SEQ ID NO: 15 - protein, 11 aa, synthetic construct
RASQGISSVLA

SEQ ID NO: 16 - protein, 7 aa, synthetic construct
DASSLES

SEQ ID NO: 17 - protein, 8 aa, synthetic construct
QQFDSSIT

SEQ ID NO: 18 - protein, 116 aa, synthetic construct
QVQLVQSGAEVKKPGSSVKVSCKASGYTFTDYNMHWVRQAPGQGLEWIGYIYPYNGGTGYNQKFKSKATITADESTNTAYMELSSLRSEDTAVYYCARGRPAMDYWGQGTLVTVSS

SEQ ID NO: 19 - protein, 111 aa, synthetic construct
DIQMTQSPSSLSASVGDRVTITCRASESVDNYGISFMNWFQQKPGKAPKLLIYAASNQGSGVPSRFSGSGSGTDFTLTISSLQPDDFATYYCQQSKEVPWTFGQGTKVEIK

SEQ ID NO: 20 - protein, 5 aa, synthetic construct
DYNMH

SEQ ID NO: 21 - protein, 17 aa, synthetic construct
YIYPYNGGTGYNQKFKS

SEQ ID NO: 22 - protein, 7 aa, synthetic construct
GRPAMDY

SEQ ID NO: 23 - protein, 15 aa, synthetic construct
RASESVDNYGISFMN

SEQ ID NO: 24 - protein, 9 aa, synthetic construct
QQSKEVPWT

SEQ ID NO: 25 - protein, 122 aa, synthetic construct
QVQLVQSGAEVKKPGESVKVSCKASGYTFTNYGMNWVKQAPGQGLEWMGWINTYTGEPTYADKFQGRVTMTTDTSTSTAYMEIRNLGGDDTAVYYCARWSWSDGYYVYFDYWGQGTSVTVSS

SEQ ID NO: 26 - protein, 113 aa, synthetic construct
DIVMTQSPDSLTVSLGERTTINCKSSQSVLDSSTNKNSLAWYQQKPGQPPKLLLSWASTRESGIPDRFSGSGSGTDFTLTIDSPQPEDSATYYCQQSAHFPITFGQGTRLEIK

SEQ ID NO: 27 - protein, 5 aa, synthetic construct
NYGMN

SEQ ID NO: 28 - protein, 17 aa, synthetic construct
WINTYTGEPTYADKFQG

SEQ ID NO: 29 - protein, 13 aa, synthetic construct
WSWSDGYYVYFDY

SEQ ID NO: 30 - protein, 17 aa, synthetic construct
KSSQSVLDSSTNKNSLA

SEQ ID NO: 31 - protein, 7 aa, synthetic construct
WASTRES

SEQ ID NO: 32 - protein, 9 aa, synthetic construct
QQSAHFPIT

SEQ ID NO: 33 - protein, 117 aa, synthetic construct
QVQLVQSGAEVKKPGASVKVSCKASGYTFTNYDINWVRQAPGQGLEWIGWIYPGDGSTKYNEKFKAKATLTADTSTSTAYMELRSLRSDDTAVYYCASGYEDAMDYWGQGTTVTVSS

SEQ ID NO: 34 - protein, 107 aa, synthetic construct
DIQMTQSPSSLSASVGDRVTINCKASQDINSYLSWFQQKPGKAPKTLIYRANRLVDGVPSRFSGSGSGQDYTLTISSLQPEDFATYYCLQYDEFPLTFGGGTKVEIK

SEQ ID NO: 35 - protein, 5 aa, synthetic construct
NYDIN

SEQ ID NO: 36 - protein, 17 aa, synthetic construct
WIYPGDGSTKYNEKFKA

SEQ ID NO: 37 - protein, 8 aa, synthetic construct
GYEDAMDY

SEQ ID NO: 38 - protein, 11 aa, synthetic construct
KASQDINSYLS

SEQ ID NO: 39 - protein, 7 aa, synthetic construct
RANRLVD

SEQ ID NO: 40 - protein, 9 aa, synthetic construct
LQYDEFPLT

SEQ ID NO: 41 - protein, 118 aa, synthetic construct
QVQLQQPGAEVVKPGASVKMSCKASGYTFTSYYIHWIKQTPGQGLEWVGVIYPGNDDISYNQKFQGKATLTADKSSTTAYMQLSSLTSEDSAVYYCAREVRLRYFDVWGQGTTVTVSS

SEQ ID NO: 42 - protein, 112 aa, synthetic construct
EIVLTQSPGSLAVSPGERVTMSCKSSQSVFFSSSQKNYLAWYQQIPGQSPRLLIYWASTRESGVPDRFTGSGSGTDFTLTISSVQPEDLAIYYCHQYLSSRTFGQGTKLEIK

SEQ ID NO: 43 - protein, 5 aa, synthetic construct
SYYIH

SEQ ID NO: 44 - protein, 17 aa, synthetic construct
VIYPGNDDISYNQKFQG

SEQ ID NO: 45 - protein, 9 aa, synthetic construct
EVRLRYFDV

SEQ ID NO: 46 - protein, 17 aa, synthetic construct
KSSQSVFFSSSQKNYLA

SEQ ID NO: 47 - protein, 8 aa, synthetic construct
HQYLSSRT

SEQ ID NO: 48 - DNA, 21 bp, synthetic construct
  source 1..21: mol_type = other DNA
taaacaccccatggatctagg

SEQ ID NO: 49 - DNA, 20 bp, synthetic construct
  source 1..20: mol_type = other DNA
gcagtacccatgaacttccc

SEQ ID NO: 50 - DNA, 20 bp, synthetic construct
  source 1..20: mol_type = other DNA
ccacaggtcaagaagaggcc

SEQ ID NO: 51 - DNA, 20 bp, synthetic construct
  source 1..20: mol_type = other DNA
ttgaccttcccttgtggctg

SEQ ID NO: 52 - skipped

SEQ ID NO: 53 - DNA, 20 bp, synthetic construct
  source 1..20: mol_type = other DNA
tatggatccaaatttctggc

SEQ ID NO: 54-55 - skipped (2 entries)

SEQ ID NO: 56 - DNA, 20 bp, synthetic construct
  source 1..20: mol_type = other DNA
tccaaatttctggctgcaag

SEQ ID NO: 57 - DNA, 20 bp, synthetic construct
  source 1..20: mol_type = other DNA
aaatttctggctgcaagtgc

SEQ ID NO: 58 - DNA, 20 bp, synthetic construct
  source 1..20: mol_type = other DNA
gatccaaatttctggctgca

SEQ ID NO: 59-61 - skipped (3 entries)

SEQ ID NO: 62 - DNA, 20 bp, synthetic construct
  source 1..20: mol_type = other DNA
agaaatttggatccatagcc

SEQ ID NO: 63 - DNA, 20 bp, synthetic construct
  source 1..20: mol_type = other DNA
agccagaaatttggatccat

SEQ ID NO: 64-66 - skipped (3 entries)

SEQ ID NO: 67 - DNA, 20 bp, synthetic construct
  source 1..20: mol_type = other DNA
gctgcaagtgcaggagtcag

SEQ ID NO: 68 - DNA, 20 bp, synthetic construct
  source 1..20: mol_type = other DNA
tggctgcaagtgcaggagtc

SEQ ID NO: 69 - skipped

SEQ ID NO: 70 - DNA, 20 bp, synthetic construct
  source 1..20: mol_type = other DNA
tgggatggaagaaagtgcag

SEQ ID NO: 71 - DNA, 20 bp, synthetic construct
  source 1..20: mol_type = other DNA
atggaagaaagtgcagggca

SEQ ID NO: 72-74 - skipped (3 entries)

SEQ ID NO: 75 - DNA, 20 bp, synthetic construct
  source 1..20: mol_type = other DNA
ttccatcccataccctacta

SEQ ID NO: 76 - skipped

SEQ ID NO: 77 - DNA, 20 bp, synthetic construct
  source 1..20: mol_type = other DNA
gtagtagggtatgggatgga

SEQ ID NO: 78 - skipped

SEQ ID NO: 79 - DNA, 20 bp, synthetic construct
  source 1..20: mol_type = other DNA
tactacgacaagaactcccc

SEQ ID NO: 80 - DNA, 20 bp, synthetic construct
  source 1..20: mol_type = other DNA
tgtcgtagtagggtatggga

SEQ ID NO: 81 - DNA, 20 bp, synthetic construct
  source 1..20: mol_type = other DNA
gtcgtagtagggtatgggat

SEQ ID NO: 82 - skipped

SEQ ID NO: 83 - DNA, 20 bp, synthetic construct
  source 1..20: mol_type = other DNA
gcactaccagagctaactca

SEQ ID NO: 84 - DNA, 20 bp, synthetic construct
  source 1..20: mol_type = other DNA
ttgcagccagaaatttggat

SEQ ID NO: 85 - DNA, 20 bp, synthetic construct
  source 1..20: mol_type = other DNA
tgcagccagaaatttggatc

SEQ ID NO: 86 - DNA, 20 bp, synthetic construct
  source 1..20: mol_type = other DNA
gcagccagaaatttggatcc

SEQ ID NO: 87 - DNA, 20 bp, synthetic construct
  source 1..20: mol_type = other DNA
cagccagaaatttggatcca

SEQ ID NO: 88 - DNA, 20 bp, synthetic construct
  source 1..20: mol_type = other DNA
agccagaaatttggatccat

(19) United States
(12) Patent Application Publication
(10) Pub. No.: US 2026/0242735 A1
(43) Pub. Date: Aug. 20, 2026

(54) DISCERNIBLE CELL SURFACE PROTEIN VARIANTS OF CD33 FOR USE IN CELL THERAPY

(71) Applicants: Universität Basel, Basel (CH); Cimeio Therapeutics AG, Basel (CH)

(72) Inventors: Stefanie URLINGER, Munich (DE); Rosalba LEPORE, Basel (CH); Mathilde TESTUT, Village Neuf (FR); Lukas JEKER, Riehen (CH); Anna CAMUS, Basel (CH); Thomas BURGOLD, Riehen (CH); Simon GARAUDE, Basel (CH); Astrid BEERLAGE, Basel (CH)

(21) Appl. No.: 19/164,484

(22) PCT Filed: Apr. 2, 2024
(86) PCT No.: PCT/EP2024/058954

§ 371 Date: Sep. 11, 2025

(30) Foreign Application Priority Data
Apr. 3, 2023 (EP) 23166347.7
Nov. 9, 2023 (EP) 23208873.2

(51) Int. Cl. C12N 5/0789; A61K 35/17; A61K 40/11; A61K 40/31; A61K 40/42; A61P 35/02; C07K 14/705; C07K 16/28; C12N 5/0783

(52) U.S. Cl. CPC C12N 5/0647; A61K 35/17; A61K 40/11; A61K 40/31; A61K 40/4224; A61P 35/02; C07K 14/70596; C07K 16/2896; C12N 5/0636; A61K 2239/13; A61K 2239/48; C07K 2317/34; C07K 2317/565; C12N 2510/00

(57) ABSTRACT

The present disclosure relates to the use of cells having discernible surface protein with engineered or naturally occurring mutation(s) but functional surface protein for use in therapy. The present invention also relates to the use of cells having discernible CD33 surface protein variants but functional surface protein for use in therapy, in particular adoptive cell therapy.

The present disclosure relates to the use of cells having discernible surface protein with engineered or naturally occurring mutation(s) but functional surface protein for use in therapy. The present invention also relates to the use of cells having discernible CD33 surface protein variants but functional surface protein for use in therapy, in particular adoptive cell therapy.

### STATEMENT REGARDING FUNDING

The project leading to this application has received funding from the European Research Council (ERC) under the European Union's Horizon 2020 research and innovation programme (grant agreement No 818806).

### BACKGROUND OF THE INVENTION

Cell based immunotherapy is emerging as the third pillar of medicine after small molecule therapy and treatments based on biologics such as recombinant proteins including antibodies. Cellular therapy can be used in oncology for treating hematopoietic malignant diseases, but also other applications such as the treatment of genetic diseases, solid organ tumors and autoimmune diseases are under development. However, cellular therapy can be associated with severe unwanted side effects. Indeed, while cancer immunotherapy with chimeric antigen receptor (CAR) T cells has been successful in targeting and eradicating malignant cells expressing a specific antigen, it does often not discriminate between normal and malignant cells and thus induces destruction of the normal hematopoietic system. Targeted therapies, which include antibody-based therapies, such as conventional monoclonal antibodies, multispecific antibodies, such as T cell engagers (e.g. BiTEs) and cellular therapies, such as CAR cells (e.g. CAR T-cells, CAR NK cells or CAR macrophages), eliminate all cells expressing the target molecule. However, most cancer cell surface antigens are shared with normal hematopoietic or other cells. Thus, to identify targets to kill diseased cells including tumors while avoiding damage to healthy cells is a major challenge for targeted therapies (Perna et al., Cancer Cell (2017) 32:506-519). In particular in myeloid diseases including myeloid malignancies such as myelodysplastic syndrome (MDS), acute myeloid leukemia (AML) or Chronic Myeloid Leukemia (CMML) cell surface antigens such as CD117, CD33, or CD123 are shared with normal myeloid progenitors and even hematopoietic stem cells. Therefore, immunotherapy targeting CD117, CD33 or CD123 antigen for MDS, AML or CMML can be associated with depletion of normal hematopoietic cells in addition to malignant cells in patients (Gill S. I. Best practice & Research Clinical Hematology, 2019). As a consequence, targeted immunotherapy including mAbs, T cell engagers or CAR T have in many cases been elusive, in part owing to the absence of truly disease-specific surface antigens (Gill S. I. Best practice & Research Clinical Hematology, 2019).

To regenerate normal hematopoiesis depleted through CD33-CAR T cell transfer, CD33 CAR T cell resistant hematopoietic cells are being engineered in such a way that the entire CD33 gene is knocked out (Kim et al. 2018. Cell. 173:1439-53). However, CD33 has a constitutive inhibitory effect on myeloid cells through its immunoreceptor tyrosine-based inhibitory motif (ITIM) signaling domain. Thus, it remains unclear how well the loss of CD33 may be tolerated (Wißfeld et al. Glia (2021) 69:1393-1412). CD33-knock-out (CD33 KO) engineered cells transplanted in patients could present long-term functional defects (WO2018/160768, Kim et al. 2018. Cell. 173:1439-53, Borot et al. 2019. PNAS. 116:11978-87, Humbert et al. 2019. Leukemia. 33:762-808). In fact, the frequency of CD33 KO cells decreased in the two monkeys for which a long-term observation was reported. This could indicate functional impairment of CD33 KO cells, for instance through reduced engraftment of CD33 KO long-term repopulating HSC (LT-HSC) or through a competitive disadvantage (Kim et al. 2018. Cell. 173:1439-53). In addition, the number of cell surface antigens with dispensable function is very limited and loss of said redundant cell surface antigen can induce antigen negative relapse. CD19-negative relapses are observed in approximately 30% of patients receiving CD19-targeted CAR T therapy (Orlando et al. 2018 Nat Med 24: 1504-6). Dual targeting of CD19 and CD123 can prevent antigen-loss relapses (Ruella et al. 2016 J Clin Invest 126:3814-26).

The inventors in previous patent applications showed that a single amino acid difference in surface protein variants can be genetically engineered into hematopoietic cells to change the antigenicity and be discriminated by specific and selective antibodies (WO2017/186718, WO2018/083071). Contrary to the approach where a surface protein is removed (KO cells), the surface protein variants in these cells retain their normal expression and function and enable to target surface proteins with important non-redundant functions.

CD33, also known as SIGLEC3 or P67, is a transmembrane receptor expressed on cells of the myeloid lineage but can also be found on some lymphoid cells, such as subsets of NK cells. The extracellular part of CD33 contains two immunoglobulin domains (one IgV and one IgC2 domain), and CD33 therefore belongs to the immunoglobulin superfamily. The intracellular part of CD33 contains certain motifs, so called immunoreceptor tyrosine-based inhibitory motifs or ITIM's, that have a role in the inhibition of cellular activity.

CD33 can be stimulated by sialic acids residues, which can for example be found on glycoproteins or glycolipids. Upon binding of sialic acid the cytosolic portion of CD33 is phosphorylated and acts as a binding site for Src homology 2 (SH2)-domain containing proteins, like SHP phosphatases. This results in a cascade that inhibits phagocytosis in the cell.

The present disclosure aimed to identify amino acid residues of CD33 that are exposed on the cell surface and that can be substituted in a manner such that a) the function of CD33 is not, or at least not substantially, altered, i.e. the variant of CD33 is functionally indistinguishable from the wild type version of CD33, and b) a moiety, such as an antibody or a CAR T cell, that binds to the wild type version of CD33, but shows a substantially decreased or no binding to the altered version of CD33, i.e. the variant of CD33 is immunologically distinguishable from the wild type version of CD33. Most single amino acid substitution in any given target protein will only affect the binding of a moiety, if the amino acid substitution is part of, or is close to, the epitope of the binding moiety. As also will be appreciated, single amino acid substitutions that do affect binding of a binding moiety to a target antigen can also impact the functionality of the target antigen. It is therefore a highly sophisticated and unpredictable task to identify those amino acid substitutions that fulfill both requirements that do affect binding a moiety to a target antigen and which at the same time do not, or not substantially, affect its function.

The present disclosure also aimed to identify amino acid residues of CD33 that are exposed on the cell surface and that can be substituted in a manner such that a) the expression of CD33 is not, or at least not substantially, altered, i.e. the level of CD33 expression is indistinguishable or substantially indistinguishable from the wild type version of CD33, and b) a moiety, such as an antibody or a CAR T cell, that binds to the wild type version of CD33, but shows a substantially decreased or no binding to the altered version of CD33, i.e. the variant of CD33 is immunologically distinguishable from the wild type version of CD33.

Several anti-CD33 moieties are known in the art. CD33 is the target of gemtuzumab ozogamicin (Mylotarg), an antibody-drug conjugate for the treatment of acute myeloid leukemia (AML) which was developed by Pfizer/Wyeth-Ayerst). CD33 is also the target of vadastuximab talirine, an antibody-drug conjugate developed by Seagen Inc. Lintuzumab is a humanized antibody against CD33 developed by Seagen Inc. for the treatment of acute myeloid leukemia (AML). BI-836858 is a fully human IgG1 anti-CD33 monoclonal antibody with antineoplastic activity developed by Boehringer Ingelheim. AMG330 is a bispecific T cell engager with specificity for CD33 and CD3 which is developed by Amgen for the treatment of AML. IMG779 is an antibody-drug conjugate against CD33 developed by ImmunoGen Inc.

### SUMMARY OF THE INVENTION

One of the objectives of the present disclosure is to develop a safer method for the treatment of malignancies, in particular cancer, hematological malignancies, and myeloid diseases. The inventors thus sought variations of the surface protein CD33, which are immunologically distinguishable while retaining or substantially retaining normal function and/or expression, and where amino acid changes originate from a single or multiple amino acid or nucleotide variations. In particular, the inventors identified variants of CD33 which change the antigenicity of CD33 to a specific antibody while retaining its normal expression and function, such as the binding to sialic acids, the stimulation by sialic acids, phosphorylation upon binding to sialic acids, the capability to act as a docking site for Src homology 2 (SH2) domain-containing proteins and/or are the capability to trigger the inhibition of phagocytosis. This was achieved via a sophisticated campaign involving a screening project, a rational design approach and a comparison to naturally occurring polymorphisms.

The present disclosure relates to a mammalian cell or a population of cells expressing a first isoform of CD33 for use in a medical treatment in a patient in need thereof, said patient having cells expressing a second isoform of CD33, wherein said cell expressing said first isoform comprises genomic DNA with at least one polymorphism or genetically engineered allele, wherein said polymorphism or genetically engineered allele is not present in the genome of the patient having cells expressing said second isoform of CD33 and preferably wherein said first and second isoform are functional. Alternatively said first isoform is generated via RNA editing.

The present disclosure also relates to a mammalian cell or a population of cells expressing a first isoform of CD33 for use in a medical treatment in a patient in need thereof, said patient having cells expressing a second isoform of CD33, wherein said cell expressing said first isoform comprises genomic DNA with at least one polymorphism or genetically engineered allele, wherein said polymorphism or genetically engineered allele is not present in the genome of the patient having cells expressing said second isoform of CD33 and preferably wherein said first and second isoform are expressed at the same level or substantially the same level.

In a particular embodiment, the present disclosure relates to the mammalian cell or population of cells, preferably hematopoietic stem cells for use in a medical treatment in a patient in need thereof wherein said medical treatment comprises: administering a therapeutically efficient amount of said cell or population of cells expressing said first isoform of CD33 to said patient in need thereof, in combination with a therapeutically efficient amount of a depleting agent comprising at least a first antigen-binding region that binds specifically to said second isoform of CD33 to specifically deplete patient cells expressing said second isoform of CD33, preferably to restore normal hematopoiesis after immunotherapy in the treatment of hematopoietic disease, and preferably in the treatment of malignant hematopoietic disease such as acute myeloid leukemia (AML), myelodysplastic syndrome (MDS), chronic myeloid leukemia (CML), chronic myelomonocytic leukemia (CMML), polycythemia vera (PV), essential thrombocythemia (ET), primary myelofibrosis (PMF), blastic plasmacytoid dendritic cell neoplasm (BPDCN) and other myeloproliferative neoplasms.

In other embodiments the medical treatment relates to the restoration of the hematopoietic or the immune function in genetic diseases of the hematopoietic or immune system, such as severe combined immunodeficiency syndrome (SCID), sickle cell disease (SCD), beta-thalassemia, Fanconi anemia or Diamond-Blackfan anemia.

In other embodiments the medical treatment relates to the restoration of the normal function in genetic diseases that are not originating in the hematopoietic and immune system but that can be treated by use of modified hematopoietic cells.

In other embodiments the medical treatment relates to the restoration of the normal immune function in autoimmune diseases, such as systemic lupus erythematosus (SLE), systemic sclerosis (SSc) or multiple sclerosis (MS).

In another particular embodiment, the present disclosure relates to the mammalian cell or population of cells for use in a medical treatment in a patient in need thereof, wherein said medical treatment comprises: administering a therapeutically efficient amount of said cell or population of cells expressing said first isoform to said patient in need thereof in combination with a therapeutically efficient amount of a depleting agent comprising at least a second antigen-binding region that binds specifically to said first isoform to specifically deplete transferred cells expressing first isoform, preferably for use in adoptive cell transfer therapy, more preferably for the treatment of malignant hematopoietic disease such as acute myeloid leukemia (AML), myeloblastic syndrome (MDS), chronic myeloid leukemia (CML), chronic myelomonocytic leukemia (CMML), polycythemia vera (PV), essential thrombocythemia (ET), primary myelofibrosis (PMF), blastic plasmacytoid dendritic cell neoplasm (BPDCN) and other myeloproliferative neoplasms, again more preferably wherein said depleting agent is administered subsequently to said cell or population of cells expressing said first isoform of surface protein to avoid eventual severe side effects such as graft-versus-host disease due to the transplantation.

In another aspect, the present disclosure relates to a pharmaceutical composition comprising a mammalian cell, preferably a hematopoietic stem cell or an immune cell such as a myeloid or NK-cell as described above and preferably a depleting agent and a pharmaceutically acceptable carrier.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33.

In certain embodiments said depleting agents binds substantially weaker to said second isoform of CD33.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform are substantially functionally identical. In certain embodiments said depleting agent binds substantially weaker to said second isoform of CD33.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform are expressed at substantially the same level. In certain embodiments said depleting agent binds substantially weaker to said second isoform of CD33.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform bind to sialic acids.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform are stimulated by sialic acids.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform act as a docking site for Src homology 2 (SH2) domain-containing proteins.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform have the capability to trigger the inhibition of phagocytosis

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform lead to essentially the same modulation of myeloid cell function and/or NK cell function.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform lead to essentially the same modulation of microglia function.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, and wherein said first and second isoform lead to the normal differentiation of hematopoietic cells.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, wherein said polymorphic or genetically engineered allele is characterized by at least one substitution of an amino acid in position N20, F21, W22, Q24, F43, F44, H45, P48, Y49 and/or Y50 of SEQ ID NO: 1, preferably an amino acid at position N20, F21, W22 and/or Y50 of SEQ ID NO: 1. In certain embodiments said depleting agents bind substantially weaker to said second isoform of CD33.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, wherein said polymorphic or genetically engineered allele is characterized by at least one substitution of an amino acid in position N20, F21, W22, Q24, F43, F44, H45, P48, Y49 and/or Y50. Among these substitutions, substitutions N20, F21, W22 and Y50 are particularly preferred. In certain embodiments said depleting agents binds substantially weaker to said second isoform of CD33.

The present disclosure also relates to a depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind to said second isoform of CD33, wherein residue N20 is substituted with G, S, D, E, K, V, R or H, residue F21 is substituted with V, L, I, S, M, N Q or H, residue W22 is substituted with S, T, E, R, D, K or H, Q24 is substituted with R, F43 is substituted with S, F44 is substituted with P or S, H45 is substituted with Y, residue P48 is substituted with F, D or S, residue Y49 is substituted with A, and/or residue Y50 is substituted with S, L, R, D, E, A, K or H.

The present disclosure also relates to a method for improving engraftment of hematopoietic stem cell transplants. Conditioning (depletion of HSCs) prior to hematopoietic stem cell transplantation (HSCT) is used to promote engraftment. Indeed, conditioning efficacy is associated with improved engraftment. Avoiding toxic conditioning is an important goal that can be achieved with the present disclosure. Current methods for conditioning involve the use of intravenous busulfan. Busulfan is a DNA alkylating drug originally designed to treat hematologic diseases, such as acute myeloid leukemia (AML). However, busulfan carries the risk of significant side effects, including sterility, primary or secondary malignancy, and additional acute and chronic toxicities.

### FIGURE LEGENDS

FIG. 1 shows the binding of anti-CD33 antibodies to HEK293-T cells transfected with human wild-type CD33 or with an empty vector. Serial dilutions of each antibody were tested for immunoreactivity via flow cytometry. All eight antibodies in Mab format bind to human CD33 in a concentration dependent manner. Cell transfected with the empty vector did not show any binding to human CD33.

FIG. 2 shows the binding of anti-CD33 Fab fragments to HEK293-T cells transfected with human wild-type CD33 or with an empty vector. Like the full-length antibodies, also the Fab fragments bind to human CD33 in a concentration dependent manner. Cells transfected with the empty vector did not show any binding to human CD33.

FIG. 3 shows the result of an alanine scan on human CD33 for six antibodies tested. For each mutant clone, the mean binding value determined by flow cytometry was plotted as a function of expression. CD33 clones with alanine substitutions that were identified as critical are circled. Secondary clones, i.e., alanine substitutions that did not meet the initially set thresholds but whose decreased binding activity and proximity to critical residues suggested that the mutated residue may be part of the antibody epitope, are squared.

FIG. 4 shows the 3D structure of CD33 with the residues identified in the alanine scan indicated for six of the antibodies tested.

FIG. 5 shows of an assessment of the surface exposure of the certain amino acid residues of CD33.

FIG. 6 depicts the binding of antibodies to the variants which show a binding to Refmab #1 of less than 10% as compared to wild type CD33.

FIG. 7 depicts the binding of antibodies to the variants which show a binding to Refmab #3 of less than 10% as compared to wild type CD33.

FIG. 8 depicts the binding of antibodies to the variants which show a binding to Refmab #4 of less than 10% as compared to wild type CD33.

FIG. 9 shows histograms of binding to Refmab #3 after base editing of human CD34+ HSPC with various sgRNAs. sgRNA-E showed the highest loss of binding to Refmab #3, followed by sgRNA-S.

FIG. 10 shows the binding of edited HSPCs to Refmab #3 as measured by flow cytometry. It can be seen that 95.8% of the non-target control cells did bind to Refmab #3, whereas 89.0% of the cells transfected with sgRNA-E did not bind to Refmab #3.

FIG. 11 confirm that successful base editing is linked to a loss of binding to Refmab #3, as determined by NGS sequencing. Essentially all edited cells (A) showed a loss of binding to Refmab #3, whereas essentially all unedited cells (C) were still reactive to Refmab #3. Only half of the intermediate cell population (B) showed the intended gene editing, consistent with a heterozygous mutation leading only to partial loss of binding.

FIG. 12 shows that edited cells differentiated to the same degree into myeloid cell and erythroid cells as unedited cells in a colony-forming unit assay, demonstrating that gene editing had no impact on the differentiation of cells.

FIG. 13 further confirms that edited HSPCs are functionally indistinguishable from unedited HSPCs, as measured in an in vitro differentiation assay.

FIG. 14 shows engraftment of edited and unedited HSPCs in NBSGW mice after 13 weeks. Mouse HSPCs are nearly completely replaced by human HSPCs (A). Absolute cell counts of hCD45+ cells are comparable in between unedited and edited cells (B). The majority of sgRNA-E edited HSPC loose binding to Refmab #3 as a surrogate for successful editing (C).

FIG. 15 shows differentiation of edited and unedited HSPC in the peripheral blood after 13 weeks, comparable numbers of myeloid cells (A), B- and T-lymphocytes (B,C), plasmacytoid and classical dendritic cells (D, E) and monocytes (F) are present.

FIG. 16 shows an outline of the experiment of Example 23.

FIG. 17 shows luminescence of mice injected with tumor cells on day 0 (A: MOLM-14, B: OCI-AML2). After treatment initiation on day 10, rapid tumor growth is seen in animals treated with isotype control or saline, whereas the tumor diminishes in animals treated with Refmab #1 and Refmab #3. Luminescence in these animals is comparable to a control mouse without tumor cell injection.

FIG. 18 show flow cytometry of peripheral blood (A, B) and bone marrow 19 days after injection of tumor cells. Refmab #1 and Refmab #3 eliminate both MOLM-14 (A,C) and OCI-AML (B,D) cells in comparison to an isotype control and saline.

FIG. 19 shows the binding of CD33 wildtype and variants to sialic acid ligands.

### DETAILED DESCRIPTION OF THE INVENTION

Immunotherapy is a promising therapy to treat cancer, genetic and autoimmune diseases. Immunodepleting agent such as antibodies or engineered immune cells directed to tumor antigen are administered into a patient to target and kill tumor cells. However, as tumor surface proteins are also expressed at the surface of normal cells including hematopoietic cells, this strategy can induce severe side effects to the patients, e.g., by altering hematopoiesis. To restore hematopoiesis in the patient, hematopoietic cells can be subsequently transplanted into the patient. However, the binding of the depleting agent not only to the diseased cells but also to the newly transplanted healthy cells can limit the maximal tolerated dose or limit the use to treatment before transplantation of healthy cells. Alternatively, transplanted cells need to be resistant to said immunodepleting agent in order not to be targeted and eliminated by it. One approach is therefore to select cells resistant to said immunodepleting agent used in immunotherapy while retaining their function to restore normal hematopoiesis in the patient.

The inventors develop a method to identify functional allelic variants in the genetic sequence encoding the surface protein region responsible for the binding of a specific depleting agent. Such variants can be naturally occurring polymorphisms and/or designed and engineered variants. Different isoforms of surface proteins can be selected or generated. Said first isoform of a surface protein encoded by a nucleic acid with said polymorphism is not recognized by a specific depleting agent. This variant allele particularly does not alter or does not substantially alter the function of the surface protein and/or is expressed at the same or substantially the same level. Thus, said depleting agent can be used to bind specifically to the one isoform and not, or not substantially, the other isoform thereby depleting specifically cells expressing one isoform. For example, if the depleting agent binds specifically to the second isoform, but not the first isoform, said depleting agent will specifically deplete cells expressing said second isoform. In another embodiment, said first isoform can be recognized by a second agent and thus this second agent can be used to deplete specifically cells expressing the first isoform, but not second isoform. The cells expressing the first isoform of the surface protein encoded by at least one variant allele is advantageously used in medical treatment in a patient having cells expressing a second isoform, in particular for depleting specifically transplanted or patient cells by using a second or first agent respectively.

It is impossible to predict, which mutation in a surface antigen can be used in such an approach. First, the mutations need to lie on a surface exposed stretch of the surface antigen that is accessible for the depleting agent. Second, the depleting agent needs to bind to this stretch on the exposed area of the surface antigen. Third, binding needs to be affected sufficiently enough so that the depleting agent can discriminate the first isoform from the second isoform. Residual binding to the other isoform should be minimal or, better, be completely absent. Fourth, the mutation should not affect, or only marginally affect, the function of the surface antigen. The mutated isoform should fulfill its biological function at least to an extent that is tolerable in a given therapeutic setting. Although certain tools exist to predict three-dimensional protein structure, only experimental testing can prove the usefulness of any given mutation.

### Depleting Agent

The present disclosure relates to an agent comprising an antigen binding region which binds specifically to one isoform of CD33 on a cell and does not bind or binds substantially weaker to another isoform of CD33. Such agent is referred to herein as “depleting agent”. Both isoforms of CD33 are functional, i.e., CD33 is functional with respect to at least one relevant property. Preferably both isoforms of CD33 have that same function, i.e., they are functionally indistinguishable, or both isoforms are expressed at the same level or substantially the same level.

The two isoforms of CD33 differ however with respect to binding to the depleting agent. The depleting agent only binds specifically to one of the isoforms of CD33. The isoforms can therefore be described as functionally identical (or functionally substantially identical), but immunologically distinguishable.

The first isoform and the second isoform of CD33 may be polymorphic alleles. Preferably, the first isoform and the second isoform of CD33 are naturally occurring polymorphic alleles. Also preferably, the first isoform and the second isoform of CD33 are single nucleotide polymorphism (SNP) alleles.

The first isoform and the second isoform of CD33 may also be genetically engineered alleles. Preferably the first isoform and the second isoform of CD33 differ by one, two, three, four or five amino acids. Most preferably the first isoform and the second isoform of CD33 differ by one amino acid.

Various methods can be used to determine the mutation that is to be introduced into CD33 to generate the second isoform. For example, mutations can be randomly inserted, followed by the functional and immunological screening of the variants generated. Alternatively, mutations can be rationally designed, for example by analysis of the secondary or tertiary protein structure of CD33.

The depleting agent comprises an antigen binding region, which binds specifically to one isoform of CD33 on a cell and does not bind or binds substantially weaker to another isoform. The depleting agent of the present disclosure can be divided into two main categories.

First, the depleting agent can be a polypeptide comprising an antigen binding region. Said polypeptide may consist of one or more polypeptide chains. Preferably said polypeptide comprising an antigen binding region is an antibody. Said polypeptide comprising an antigen binding region may also be an antibody fragment, an antibody drug conjugate, or another variant of an antibody or scaffold. Exemplary antibody fragments and scaffolds include single domain antibodies, maxibodies, minibodies, intrabodies, diabodies, triabodies, tetrabodies, v-NAR, igNAR, bis-scFv, camelid antibodies, ankyrins, centyrins, domain antibodies, lipocalins, small modular immuno-pharmaceuticals, maxybodies, Protein A and affilins.

Said depleting agent can also be coupled to chemical drug, such as a cytotoxic payload or a proteolysis targeting chimera (PROTAC).

Said polypeptide comprising an antigen binding region may also be a bispecific, biparatopic or multispecific antibody. Such molecules may also contain additional functional domains. For example, said polypeptide comprising an antigen binding region may be a T cell engager, for example a BiTE. Said polypeptide comprising an antigen binding region may also be fused to a cytokine or a chemokine, a toxin or to the extracellular domain of a cell surface receptor.

Alternative, the depleting agent can be a cell comprising an antigen binding region. For example, the depleting agent can be a chimeric antigen receptor (CAR). In certain embodiments of the present disclosure said cell comprising an antigen binding region is a CAR T-cell, CAR NK cells or CAR macrophages. In a preferred embodiment of the present disclosure said cell comprising an antigen binding region is a CAR T-cell. In another preferred embodiment of the present disclosure said cell comprising an antigen binding region is a primary T cell comprising a CAR.

The depleting agent binds specifically to one isoform of CD33, but not the second isoform and thus specifically depletes cells expressing one isoform.

In certain embodiments, the present disclosure relates to an agent comprising a first antigen binding region which binds specifically to a second isoform of CD33 and does not bind a first isoform. In other embodiments, the present disclosure also relates to an agent comprising a second antigen binding region which binds specifically to the first isoform of CD33 and does not bind a second isoform. In certain embodiments said agents binds substantially weaker to said second isoform of CD33.

The first and the second isoform of CD33 may differ from each other by only one amino acid substitution. Said one amino acid difference between the first and the second isoform may also be the result of the presence of a single nucleotide polymorphism, such as a naturally occurring single nucleotide polymorphism. The first and the second isoform of CD33 may also differ from each other by more than one amino acid, such as by two, by three or by more than three amino acids. The first and the second isoform of CD33 may also differ from each other in that one of the isoforms has an insertion of one, of two, of three or of more than three amino acids compared to the other isoform. The first and the second isoform of CD33 may also differ from each other in that one of the isoforms has a deletion of one, of two, of three or of more than three amino acids compared to the other isoform. The two isoforms may also differ from each other by combinations of amino acid substitutions, insertions and/or deletions. In a preferred embodiment, said depleting agent is an antibody or an antigen-binding fragment. If the two isoforms of CD33 differ by more than one amino acid, then the amino acids changed may be adjacent to each other, i.e., direct neighboring amino acids, or they may be separated.

The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments as well as variants (including derivatives) of antibodies.

In natural antibodies of rodents and primates, two heavy chains are linked to each other by disulfide bonds, and each heavy chain is linked to a light chain by a disulfide bond. There are two types of light chains, lambda (λ) and kappa (κ). There are five main heavy chain classes (or isotypes) which determine the functional activity of an antibody molecule: IgM, IgD, IgG, IgA and IgE. Each chain contains distinct sequence domains. In typical IgG antibodies, the light chain includes two domains, a variable domain (VL) and a constant domain (CL). The heavy chain includes four domains, a variable domain (VH) and three constant domains (CH1, CH2 and CH3, collectively referred to as CH). The variable regions of both light (VL) and heavy (VH) chains determine binding recognition and specificity to the antigen. The constant region domains of the light (CL) and heavy (CH) chains confer important biological properties such as antibody chain association, secretion, trans-placental mobility, complement binding, and binding to Fc receptors (FcR).

The Fv fragment is the N-terminal part of the Fab fragment of an immunoglobulin and consists of the variable portions of one light chain and one heavy chain variable region. The specificity of the antibody resides in the structural complementarity between the antibody combining site and the antigenic determinant. Antibody combining sites are made up of residues that are primarily from the hypervariable or complementarity determining regions (CDRs). Occasionally, residues from non-hypervariable or framework regions (FR) can participate in the antibody binding site or influence the overall domain structure and hence the combining site. Complementarity Determining Regions or CDRs refer to amino acid sequences which together define the binding affinity and specificity of the natural Fv region of a native immunoglobulin binding site. The light and heavy chains of an immunoglobulin each have three CDRs, designated L-CDR1, L-CDR2, L-CDR3 and H-CDR1, H-CDR2, H-CDR3, respectively. An antigen-binding site, therefore, typically includes six CDRs, comprising the CDRs set from each of a heavy and a light chain V region. Framework Regions (FRs) refer to amino acid sequences interposed between CDRs. Accordingly, the variable regions of the light and heavy chains typically comprise 4 framework regions and 3 CDRs of the following sequence: FR1-CDR1-FR2-CDR2-FR3-CDR3-FR4.

The residues in antibody variable domains are conventionally numbered according to a system devised by Kabat et al. This system is set forth in Kabat et al., 1987, in Sequences of Proteins of Immunological Interest, US Department of Health and Human Services, NIH, USA (Kabat et al., 1992, hereafter “Kabat et al.”). This numbering system is used in the present specification. The Kabat residue designations do not always correspond directly with the linear numbering of the amino acid residues in SEQ ID sequences. The actual linear amino acid sequence may contain fewer or additional amino acids than in the strict Kabat numbering corresponding to a shortening of, or insertion into, a structural component, whether framework or complementarity determining region (CDR), of the basic variable domain structure. The correct Kabat numbering of residues may be determined for a given antibody by alignment of residues of homology in the sequence of the antibody with a “standard” Kabat numbered sequence. The CDRs of the heavy chain variable domain are located at residues 31-35 (H-CDR1), residues 50-65 (H-CDR2) and residues 95-102 (H-CDR3) according to the Kabat numbering system. The CDRs of the light chain variable domain are located at residues 24-34 (L-CDR1), residues 50-56 (L-CDR2) and residues 89-97 (L-CDR3) according to the Kabat numbering system.

In specific embodiments, an antibody provided herein is an antibody fragment, and more particularly any protein including an antigen-binding domain of an antibody as disclosed herein. The antigen-binding domain may also be integrated into another protein scaffold Antibody fragments and scaffolds include, but are not limited to, Fv, Fab, F(ab′)2, Fab′, dsFv, scFv, sc(Fv)2, diabodies, single domain antibodies, maxibodies, minibodies, intrabodies, diabodies, triabodies, tetrabodies, v-NAR, IgNAR, bis-scFv, camelid antibodies, ankyrins, centyrins, domain antibodies, lipocalins, small modular immuno-pharmaceuticals, maxybodies, Protein A and affilins.

As used herein, an “antigen binding region” or “antigen-binding fragment of an antibody” means a part of an antibody, i.e. a molecule corresponding to a portion of the structure of the antibody, that exhibits antigen-binding capacity for a specific antigen, possibly in its native form; such fragment especially exhibits the same or substantially the same antigen-binding specificity for said antigen compared to the antigen-binding specificity of the corresponding four-chain antibody. The antigen-binding capacity can be determined by measuring the affinity between the antibody and the target fragment. This antigen-binding region may also be designated as “functional fragments” of antibodies.

The agents of the disclosure comprise antibodies and fragments thereof but also comprise artificial proteins with the capacity to bind antigens mimicking that of antibodies, also termed herein antigen-binding antibody mimetic. Antigen-binding antibody mimetics are organic compounds that specifically bind antigens, but that are not structurally related to antibodies. They are usually artificial peptides or small proteins with a molar mass of about 3 to 20 kDa.

The phrases “an antigen binding region recognizing an antigen” and “an antigen binding region having specificity for an antigen” are used interchangeably herein with the term “an antigen binding region which binds specifically to an antigen”. As used herein, the term “specificity” refers to the ability of an agent comprising an antigen binding region such as an antibody to detectably bind an epitope presented on an antigen.

“Specific binding” or “specifically bind to” includes binding with a monovalent affinity of about 10⁻⁸ M (KD) or stronger. Preferably, binding is considered specific when the binding affinity is between 10⁻⁸ M (KD) and 10⁻¹² M (KD), optionally between 10⁻⁸ M (KD) and 10⁻¹ M (KD), in particular at least 10⁻⁸ M (KD). The affinity can be determined by various methods well known from the one skilled in the art. These methods include, but are not limited to, surface plasmon resonance (SPR), biolayer interferometry (BLI), microscale thermophoresis (MST) and Scatchard plot. Whether a binding domain specifically reacts with or binds to a target can be tested readily by, inter alia, comparing the reaction of said binding domain with a target protein or antigen with the reaction of said binding domain with proteins or antigens other than the target protein.

As used herein, the term “epitope” means the part of an antigen to which the antibody or antigen binding region thereof binds. The epitopes of protein antigens can be divided into two categories, conformational epitope and linear epitope. A conformational epitope corresponds to discontinuous sections of the antigen's amino acid sequence. A linear epitope corresponds to a continuous sequence of amino acids from the antigen.

In another aspect, it is further disclosed herein bispecific or multispecific molecules, such as bispecific antibodies or multispecific antibodies. For example, an antibody can be derivatized or linked to another functional molecule, e.g., another peptide or protein (e.g., another antibody or ligand for a receptor) to generate a bispecific molecule that binds to at least two different binding sites or target molecules. The antibody may in fact be derivatized or linked to more than one other functional molecule to generate multi-specific molecules that bind to more than two different binding sites and/or target molecules; such multi-specific molecules are also intended to be encompassed by the terms “bispecific molecule”, “bispecific antibody”, “biparatopic molecule”, “biparatopic antibody”, “multispecific molecule” and “multispecific antibody” as used herein. To create a bispecific molecule, an antibody of the disclosure can be functionally linked (e.g., by chemical coupling, genetic fusion, disulfide bonds, noncovalent association or otherwise) to one or more other binding molecules, such as another antibody, antibody fragment, peptide or binding mimetic, cytokine, chemokine, toxin, PROTAC or a receptor extracellular domain, such that a bispecific molecule results. Specific bispecific and multispecific molecules contemplated by the present disclosure are T cell engagers, such as bispecific T cell engager, for example a BiTE.

As used herein, an agent which does not bind or binds substantially weaker to a particular isoform of CD33 includes an agent which is not able to bind to cells expressing said particular isoform. For experimental testing said agent may be labelled with a fluorescent marker or may be detected with a secondary antibody directed against said agent, and the percentage of cells presenting said fluorescent marker or said secondary antibody is determined by FACS analysis. Typically testing is done in cell lines expressing the recombinant target protein, i.e. CD33. The target protein may be expressed in its entirety. Alternatively, a truncated version may be used, wherein said truncated version at a minimum needs to include the extra cellular domain or the regions of the extracellular domain containing the respective antibody epitope. In order to monitor the expression of the variant isoforms, cells may be stained with two agents simultaneously, one binding the epitope where variants were introduced and a second one that binds an epitope that is different from the one bound by the first agent. The second epitope remains unaltered and thus this staining serves as an expression control. As a non-binding control, cells are used that do not express the protein of interest. As a maximum binding control, cells that normally do not express the protein of interest are transfected with the wildtype isoform. Different cell lines have different expression levels, but the expression is controlled through endogenous control elements such as promoters. Such cell lines can also be used to study the mode-of-action of a depleting agent, the effective shielding against a different mode-of-action, to test cytotoxicity and shielding/resistance from cytotoxicity or to test the function of the engineered receptors. Western Blot, ELISA or FACS can be used to analyze phosphorylation of signaling molecules. Analysis of gene expression changes can serve to analyse gene expression compared to normal function. Cells can also be used to demonstrate the feasibility of editing a specific variant via different approaches, e.g., homology directed repair (HDR), base editing or prime editing.

Binding of said agent can result in depletion of the cell expressing the first isoform of CD33. Various mechanisms can lead to cell depletion. Antibody dependent cellular cytotoxicity (ADCC) results from binding of the agent to a target protein and activation of NK cells through the Fc part on the agent bound by an FcR expressed by NK cells. The Fc part of an immunoglobulin refers to the C-terminal region of an immunoglobulin heavy chain. The Fc part can be wildtype or engineered. Mutations of enhanced, engineered Fc parts are known in the art. For certain therapeutic situations, it is desirable to reduce or abolish the normal binding of the wild-type Fc region of an antibody, such as of a wild-type IgG Fc region to one or more or all of Fc receptors and/or binding to a complement component, such as C1 q in order to reduce or abolish the ability of the antibody to induce effector function. For instance, it may be desirable to reduce or abolish the binding of the Fc region of an antibody to one or more or all of the Fcy receptors, such as: FcγRI, FcγRIIa, FcγRIIb, FcγRIIIa. Effector function can include, but is not limited to, one or more of the following: complement dependent cytotoxicity (CDC), antibody-dependent cell-mediated cytotoxicity (ADCC), antibody-dependent cellular phagocytosis (ADCP), cytokine secretion, immune complex-mediated antigen uptake by antigen-presenting cells, binding to NK cells, binding to macrophages, binding to monocytes, binding to polymorphonuclear cells, direct signaling inducing apoptosis, crosslinking of target-bound antibodies, dendritic cell maturation, or T cell priming. Binding of said agent may also lead to the blocking of binding of the natural receptor ligand and thereby result in cell death and apoptosis without cell-mediated depletion.

A reduced or abolished binding of an Fc region to an Fc receptor and/or to C1q is typically achieved by mutating a wild-type Fc region, such as of an IgG1 Fc region, more particular a human IgG1 Fc region, resulting in a variant or engineered Fc region of said wild-type Fc region, e.g., a variant human IgG1 Fc region. Substitutions that result in reduced binding can be useful. For reducing or abolishing the binding properties of an Fc region to an Fc receptor, non-conservative amino acid substitutions, i.e., replacing one amino acid with another amino acid having different structural and/or chemical properties and/or charges, are preferred.

In certain embodiments of the present disclosure the Fc region of the antibody is of the IgG1 isotype, carrying mutations, i.e. the constant region carries a L234A, a L235A and a P329G mutation (PG-LALA mutations), i.e. the constant region carries a L234A, a L235A and a P329A mutation (PA-LALA mutations), or i.e. the constant region carries a L234A, a L235E, G237A, A330S and a P331S mutation (AEASS mutations). The skilled person will be aware of possibilities to engineer the Fc region to obtain a desired effect.

Surrogate ADCC assays constitute an industry standard to quantitate an agent's potency to mediate ADCC as described in the experimental part. Engineered Jurkat reporter cells carry an NFAT-responsive luciferase gene and an Fc receptor, such as human FcgRI, FcgRIIa or FcgRIIIa. Binding of the Fc receptor to bound antibody results in NFAT induction through receptor clustering and therefore a luciferase signal. Absence of binding and therefore clustering does not result in a luciferase signal. Cells expressing either no target protein (e.g. HEK or chicken DF-1 cells) or human hematopoietic cancer cells such as TF-1, KG-1, KASUMI-1, U937, THP-1 or MV4-11 engineered to be CD33-deficient (e.g. a CD33 knock-out), or expressing the wildtype protein (e.g. HEK-CD33 or DF-1− CD33, or TF-1, KG-1, KASUMI-1, U937, THP-1 or MV4-11 cell lines) or expressing individual variants (e.g. CD33 variants) were incubated with the test agent (e.g. antibody Refmab #1, Refmab #3 or Refmab #4) and mixed with the ADCC reporter cells. Then luciferase was measured to quantify the ADCC signal. The luciferase luminescence signals were normalized to the maximal signal observed in HEK-CD33, DF-1− CD33 or the corresponding myeloid or T cell cancer cell line. ADCC was measured with an ADCC Reporter Assay (Promega, Cat. No. G7015).

Other potential modes-of-action in line with the present disclosure are possible as well. This includes antibody internalization in conjunction with the use of an antibody drug conjugate. An alternative way of depleting target cells is through the use of T cell engager molecules. For example, a bispecific T cell engager using a CD33 binding site derived from antibody Refmab #1, Refmab #3 or Refmab #4 and a CD3 (OKT3) binding site may be used. The same target cells as used for the ADCC assay are used. Primary human T cells and the bispecific T cell engager are added. Activation of human T cells is quantified by FACS by determining the frequency of, e.g., CD25 or CD69 upregulation and/or cytokine release, or T-cell mediated cell killing.

The depleting agent according to the present disclosure binds specifically to one isoform of CD33 and allows the depletion of cells expressing said isoform.

More preferably, in specific embodiments, said depleting agent according to the present disclosure does not bind or binds substantially weaker to a first isoform of CD33 but binds specifically to a second isoform of CD33 and allows the depletion of said cells expressing said second isoform of CD33, in particular in methods of use as disclosed herein. In particular, said depleting agent which does not bind or binds substantially weaker to a first isoform of CD33 but binds specifically to a second isoform of CD33 expressed in patient's cell is used to deplete patient's cells but not hematopoietic stem cells or their progeny expressing said first isoform of CD33 transplanted to restore hematopoiesis in said patient.

In another specific embodiments, said depleting agent according to the present disclosure does not bind or binds substantially weaker to a second isoform of CD33 but binds specifically to a first isoform of CD33 and allows the depletion of cells expressing said first isoform of CD33, in particular in methods of use as disclosed herein. In particular, said depleting agent which does not bind or binds substantially weaker to a second isoform of CD33 but binds specifically to a first isoform of CD33 expressed in transplanted cells is used to deplete specifically transplanted cells to avoid eventual severe side effects such as graft-versus-host disease due to transplantation.

Selective depletion of cells expressing a specific isoform of CD33 can be achieved without limitation by complement-dependent cytotoxicity (CDC), antibody-dependent cellular cytotoxicity (ADCC) or antibody-dependent cellular phagocytosis (ADCP).

In certain embodiments, the antigen binding region is coupled to an effector compound such as a drug or a toxin. Such conjugates are referred to herein as “immunoconjugates”, “antibody-drug conjugates” or “ADCs”. A cytotoxin or cytotoxic agent includes any agent that is detrimental to (e.g., kills) cells. Examples include taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, maytansinoids, calicheamicins, indolinobenzodiazepines, pyrolobenzodiazepines, pyrridinobenzodiazepines, camptothecins, topotecan, irinotecan, belotecan, deruxtecan, alpha-amanitin, microcystins, auristatins and puromycin and analogs or homologs thereof.

In certain embodiments, the antigen binding region is coupled to a PROTAC. The term “PROTAC” as used herein refers to proteolysis-targeting chimeras. PROTAC generally have three components—an E3 ubiquitin ligase binding group (E3LB), a linker, and a protein binding group. PROTACs and PROTAC binding domains are known to the skilled person (see e.g. An et al, EBioMedicine. 2018 October; 36: 553-562).

In another particular embodiment, said depleting agent is an immune cell harboring an antigen receptor such as a chimeric antigen receptor (CAR). See for example Myburgh et al. Leukemia (2020) 34: 2688-703. Said immune cell may express a recombinant antigen binding region, also named antigen receptor on its cell surface.

By “recombinant” is meant an antigen binding region which is not encoded by the cell in its native state, i.e. it is heterologous, non-endogenous. Expression of the recombinant antigen binding region can thus be seen to introduce a new antigen specificity to the immune cell, causing the cell to recognise and bind a previously unrecognised antigen. The antigen receptor may be isolated from any useful source. In certain embodiments of the present disclosure said cell comprising an antigen binding region is a CAR T-cell, a CAR NK cell, CAR Treg or a CAR macrophage. In a preferred embodiment of the present disclosure said cell comprising an antigen binding region is a CAR T-cell. In another preferred embodiment of the present disclosure said cell comprising an antigen binding region is a primary T cell comprising a CAR.

In a particular embodiment, said recombinant antigen receptor is a chimeric antigen receptor (CAR). CARs are fusion proteins comprising an antigen-binding region, typically derived from an antibody, linked to the signaling domain of the TCR complex. CARs can be used to direct immune cells such T-cells or NK cells against a target antigen, if a suitable antigen-binding region is selected.

The antigen-binding region of a CAR is typically based on a scFv (single chain variable fragment) derived from an antibody. In addition to an N-terminal, extracellular antibody-binding region, CARs typically may comprise a hinge domain, which functions as a spacer to extend the antigen-binding region away from the plasma membrane of the immune effector cell on which it is expressed, a transmembrane (TM) domain, an intracellular signaling domain (e.g. the signaling domain from the zeta chain of the CD3 molecule (CD3) of the TCR complex, or an equivalent) and optionally one or more co-stimulatory domains which may assist in signaling or functionality of the cell expressing the CAR. Signaling domains from co-stimulatory molecules including CD28, OX-40 (CD134), CD27, ICOS and 4-1BB (CD137) can be added alone (second generation) or in combination (third generation) to enhance survival and increase proliferation of CAR modified immune cells.

The skilled person is able to select an appropriate antigen binding region as described above with which to redirect an immune cell to be used according to the disclosure. In a particular embodiment, the immune cell for use in the method of the disclosure is a redirected T-cell, e.g., a redirected CD8+ T-cell or a redirected CD4+ T-cell, or a redirected NK cell.

Methods by which immune cells can be genetically modified to express a recombinant antigen binding region are well known in the art. A nucleic acid molecule encoding the antigen receptor may be introduced into the cell in the form of, e.g., a vector, or any other suitable nucleic acid construct, or by inserting the nucleic acid molecule into the genome using genome editing technologies. Vectors, and their required components, are well known in the art. Nucleic acid molecules encoding antigen binding region can be generated using any method known in the art, e.g., molecular cloning using PCR. Antigen binding region sequences can be modified using commonly used methods, such as site-directed mutagenesis.

### CD33

CD33 (UniProt: P20138; also known as SIGLEC3, SIGLEC-3, P67, sialic acid-binding Ig-like lectin 3, FL00391 or Gp67), is a transmembrane receptor expressed on cells of mainly the myeloid lineage. CD33 binds sialic acids and therefore is a member of the SIGLEC family of lectins. The extracellular portion of this receptor contains two immunoglobulin domains (one IgV and one IgC2 domain), placing CD33 within the immunoglobulin superfamily. The intracellular portion of CD33 contains immunoreceptor tyrosine-based inhibitory motifs (ITIMs) that are implicated in inhibition of cellular activity. CD33 can be stimulated by molecules with sialic acid residues, such as glycoproteins or glycolipids. Upon binding, the immunoreceptor tyrosine-based inhibition motif (ITIM) of CD33, present on the cytosolic portion of the protein, is phosphorylated and acts as a docking site for Src homology 2 (SH2) domain-containing proteins like SHP phosphatases. This results in a cascade that inhibits phagocytosis in the cell.

Human CD33 has the following amino acid sequence (SEQ ID No. 1):
MPLLLLLPLLWAGALAMDPNFWLQVQESVTVQEGLCVLVPCTFFHPIPYYDKNSPVHGYW
FREGAIISRDSPVATNKLDQEVQEETQGRFRLLGDPSRNNCSLSIVDARRRDNGSYFFRM
ERGSTKYSYKSPQLSVHVTDLTHRPKILIPGTLEPGHSKNLTCSVSWACEQGTPPIFSWL
SAAPTSLGPRTTHSSVLIITPRPQDHGTNLTCQVKFAGAGVTTERTIQLNVTYVPQNPTT
GIFPGDGSGKQETRAGVVHGAIGGAGVTALLALCLCLIFFIVKTHRRKAARTAVGRNDTH
PTTGSASPKHQKKSKLHGPTETSSCSGAAPTVEMDEELHYASLNFHGMNPSKDTSTEYSE
VRTQ

In a particular embodiment, said surface protein is CD33. In other embodiments said surface protein is CD33 comprising the amino acid sequence of SEQ ID No. 1. In other embodiments said surface protein is CD33 consisting of the amino acid sequence of SEQ ID No. 1.

In certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and preferably wherein said first and second isoform are functional.

In certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein, wherein said first and second isoform are functional.

In certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein, wherein said first and second isoform are substantially functionally identical.

Knowledge about the exact function of CD33 is still limited, although certain activities of CD33 have been described. In certain embodiments the present disclosure related to a first and a second isoform of CD33 wherein both isoforms are functional. In certain embodiments the present disclosure related to a first and a second isoform of CD33 wherein both isoforms are functionally indistinguishable. In the present invention “functionally indistinguishable” refers to a first and a second isoform of CD33 that are equally capable of performing the same function within a cell without significant impairment. In other words, the first and the second isoform are functionally largely indistinguishable. A slight functional impairment may be acceptable. In a preferred embodiment, said first isoform of CD33 remains functional and retain the capacity of performing the same function as the corresponding wild type isoform within a cell without significant impairment.

One function of CD33 is the binding of sialic acids. Therefore, in certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform bind sialic acids.

CD33 is stimulated by molecules comprising sialic acids. Therefore, in certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform are stimulated by a molecule comprising sialic acids.

Upon binding of sialic acids the immunoreceptor tyrosine-based inhibition motif (ITIM) of CD33 is phosphorylated. Therefore, in certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform are phosphorylated upon binding of sialic acids. In certain embodiments, said phosphorylation of CD33 occurs at the immunoreceptor tyrosine-based inhibition motif (ITIM) of CD33.

Upon phosphorylation, CD33 can acts as a docking site for Src homology 2 (SH2) domain-containing proteins like SHP phosphatases. Therefore, in certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform upon phosphorylation can acts as a docking site for Src homology 2 (SH2) domain-containing proteins. In certain embodiments, said Src homology 2 (SH2) domain-containing protein is a SHP phosphatase.

Upon phosphorylation, CD33 can also trigger a cascade that inhibits phagocytosis. Therefore, in certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform can inhibit phagocytosis. In certain embodiments, phagocytosis is inhibited upon phosphorylation of CD33.

In certain embodiments the present disclosure relates to a mammalian cell or a population of cells expressing a first isoform of the surface protein CD33 for use in a medical treatment in a patient in need thereof, said patient having cells expressing a second isoform of said surface protein, wherein said cell expressing said first isoform comprises genomic DNA with at least one polymorphism or genetically engineered allele, wherein said polymorphism or genetically engineered allele is not present in the genome of the patient having cells expressing said second isoform of said surface protein and wherein said first and said second isoform are expressed substantially to the same degree. In certain embodiments, said first and said second isoform are expressed to the same degree.

In line with the present disclosure, it is also possible to combine additional variants or isoforms of CD33 within the methods and compositions of the present disclosure. Such isoforms may for example include double mutants. Such isoforms may for example also include single and double mutants. The methods and compositions of the present disclosure may also be used in the depletion of myeloid cells in solid tumors in order to enhance tumor responses.

The methods and compositions of the present disclosure may also be combined with cells combinations, in particular, when said surface protein is CD33 with knock out of other targets, such as CD117, CD123, DLL-1, CD45, CD47, CD7, CLEC12A, CD44, FLT3, CD300LF, EVI2B, TPO and combinations thereof.

The methods and compositions of the present disclosure may also comprise cells expressing first isoform of CD33 and other surface protein variants such as CD117 variants, CD123 variants, DLL-1 variants, CD45 variants, CD47 variants, CD7 variants, CLEC12A (CD371) variants, CD44 variants, FFLT3 (CD135) variants, CD300LF variants, EVI2B variants, TPO variants and any combination thereof.

### Polymorphism of CD33

The cell expressing the first isoform of CD33 according to the present disclosure comprises genomic DNA with at least one polymorphic allele in the nucleic acid encoding said CD33 isoform. In particular, said polymorphism induces at least one mutation involved in the binding of a specific agent in comparison to said second isoform.

Said polymorphism is preferably within a nucleic acid sequence encoding the surface protein region of CD33 involved in binding of the first agent, preferably located in the extracellular portion of CD33, in particular, in a solvent-exposed secondary structure element. More particularly, said polymorphism is within a nucleic acid sequence encoding at least one specific amino acid residue involved in binding of the first agent. Said polymorphism can be a mutation such as a deletion, a substitution, an insertion, or a combination thereof of at least 1, 2, 3, 4, 5, 6, 7, 8, 9, 10, 12, 15 or 20 nucleotides. In a particular embodiment, said polymorphism is a single nucleotide polymorphism.

The term “isoform” refers to a variant of a protein which differs from another variant of the same protein by at least one amino acid difference. In the context of the present disclosure such difference may be a substitution of a single amino acid, but such differences may also be double, triple or multiple amino acid substitutions, or insertions or deletions. Also naturally occurring SNPs are isoforms.

The difference in the sequence of the two isoforms may also be genetically introduced. Also, here the sequence difference is preferably within a nucleic acid sequence encoding the CD33 region involved in binding of the first agent, preferably located in the extracellular portion of said surface protein, in particular, in a solvent-exposed secondary structure element. More particularly, said sequence difference is within a nucleic acid sequence encoding at least one specific amino acid residue involved in binding of the first agent. Said sequence difference can be a mutation such as a deletion, a substitution, an insertion or a combination thereof of at least 1, 2, 3, 4, 5, 6, 7, 8, 9, 10, 12, 15 or 20 nucleotides. In a particular embodiment, said sequence difference is a single point mutation.

The present disclosure provides polymorphisms in CD33, including in particular polymorphisms including substitution of the residues N20, F21, W22, Q24, F43, F44, H45, P48, Y49 and/or Y50. Particular preferred polymorphisms include substitution of the residues N20, F21, W22 and/or Y50.

Particular preferred polymorphisms include substitutions of the residue N20, wherein N20 is substituted with G, S, D, E, K, V, R or H. Other preferred polymorphisms include substitutions of the residue F21, wherein F21 is substituted with V, L, I, S, M, N, Q or H. Other preferred polymorphisms include substitutions of the residue W22, wherein W22 is substituted with S, T, E, R, D, K or H. Other preferred polymorphisms include substitutions of the residue Q24, wherein Q24 is substituted with R. Other preferred polymorphisms include substitutions of the residue F43, wherein F43 is substituted with S. Other preferred polymorphisms include substitutions of the residue F44, wherein F44 is substituted with P or S. Other preferred polymorphisms include substitutions of the residue H45, wherein H45 is substituted with Y. Other preferred polymorphisms include substitutions of the residue P48, wherein P48 is substituted with F, D or S. Other preferred polymorphisms include substitutions of the residue Y49, wherein Y49 is substituted with A. Other preferred polymorphisms include substitutions of the residue Y50, wherein Y50 is substituted with S, L, R, D, E, A, K or H. Other preferred polymorphisms include substitutions of the residue Y49 and Y50, wherein Y49 is substituted with A and Y50 is substituted with A.

In certain embodiments the present disclosure relates to a variant CD33 polypeptide, wherein said variant CD33 polypeptide comprises at least one mutation in an amino selected from N20, F21, W22, Q24, F43, F44, H45, P48, Y49 and/or Y50 of wild type human CD33. In certain embodiments the present disclosure relates to a variant CD33 polypeptide, wherein said variant CD33 polypeptide comprises at least one mutation in an amino selected from N20, F21, W22 and/or Y50 of wild type human CD33.

It will be appreciated that amino acid may be designated by the 3-letter code or the 1-letter code, which both are familiar to the skilled person.

Table 1 shows the 20 natural occurring amino acids:

| Amino acid | Three letter code | One letter code |
|---|---|---|
| alanine | Ala | A |
| arginine | Arg | R |
| asparagine | Asn | N |
| aspartic acid | Asp | D |
| cysteine | Cys | C |
| glutamic acid | Glu | E |
| glutamine | Gln | Q |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

In the experiments of the present disclosure certain variants of specific residues were identified. For practical reasons it is impossible to test any and all possible variants. It will however be understood that an identified variant may be substituted with a similar amino acid residue. For example, an acidic amino acid can be replaced by another acidic amino acid, since it can be expected to have the same effect. Likewise, a charged amino acid can be replaced by another charged amino acid. As an example, T264E is expected to be equivalent to T264D, since both, D and E are acidic amino acids.

### Natural Polymorphism

In a particular embodiment, said cell according to the present disclosure is selected from a subject comprising native genomic DNA with at least one natural polymorphism allele, preferably single nucleotide polymorphism (SNP) in the nucleic acid encoding said isoform.

In a particular embodiment, cells are selected from a subject that comprises native genomic DNA with at least one natural polymorphism allele, in particular SNP, in a nucleic acid sequence encoding CD33 region involved in anti-CD33 agent binding, preferably located in the extracellular portion of said surface protein, more preferably in a solvent-exposed secondary structure element.

Certain naturally occurring SNPs are described in the literature. These naturally SNPs may be used within the spirit of the present disclosure with a respective binding agent which is able to discriminate such SNP from another isoform of CD33. A list of natural occurring SNPs of CD33 can be found in any respective database, such as gnomAD (https://gnomad.broadinstitute.org/), dbSNP (https://www.ncbi.nlm.nih.gov/snp/=) or GeneCards (https://www.genecards.org/).

### Gene Editing

In another particular embodiment, said cell expressing the first isoform of CD33 according to the present disclosure is obtained by gene editing, preferably by changing the sequence encoding said surface protein in the patient's native genomic DNA.

The cell can be genetically engineered by introducing into the cell a gene editing system to induce said polymorphism resulting in insertion, deletion and/or substitution of amino acids of the surface protein. Said gene editing modality targets a nucleic acid sequence, named herein target sequence encoding surface protein region involved in first agent binding as described above. In particular, when said surface protein is CD33, said gene editing modality targets a nucleic acid encoding at least one amino acid residue in position N20, F21, W22, Q24, F43, F44, H45, P48, Y49 and/or Y50 of SEQ ID NO: 1. Preferably amino acid residue N20 is substituted with G, S, D, E, K, V, R or H, and/or residue F21 is substituted with V, L, I, S, M, N Q or H, and/or residue W22 is substituted with S, T, E, R, D, K or H, and/or residue Q24 is substituted with R, and/or residue F43 is substituted with S, and/or residue F44 is substituted with P or S, and/or residue H45 is substituted with Y, and/or residue P48 is substituted with F, D or S, and/or residue Y49 is substituted with A or D, and/or residue Y50 is substituted with S, L, R, D, E, K, A or H. Gene editing enzymes may be sequence-specific nucleases, base editors, prime editors or CRISPR-transposon based systems.

The term “nuclease” refers to a wild type or variant enzyme capable of catalyzing the hydrolysis (cleavage) of phosphodiester bonds between nucleotides of a nucleic acid (DNA or RNA) molecule, preferably a DNA molecule. By “cleavage” is intended a double-strand break or a single-strand break event.

The term “sequence-specific nuclease” refers to a nuclease which cleaves nucleic acid in a sequence-specific manner. Different types of site-specific nucleases can be used, such as Meganucleases, TAL-nucleases (TALEN), Zing-finger nucleases (ZFN), or RNA/DNA guided endonucleases like Clustered Regularly Interspaced Short Palindromic Repeats (CRISPR)/Cas system and Argonaute (Review in Li et al., Nature Signal transduction and targeted Therapy, 5, 2020; Guha et al., Computational and Structural Biotechnology Journal, 2017, 15, 146-160).

According to the present disclosure, the nuclease generates a DNA cleavage within a target sequence, said target sequence encodes a surface protein region involved in first agent binding as described above. In particular embodiments, the inventors use the CRISPR system to induce a cleavage within a target sequence encoding surface protein region recognized by first agent as described above.

By “target sequence”, it is intended targeting a part of the sequence encoding the region on CD33 involved in first agent binding as described as described above and/or sequences adjacent to said region on CD33 involved in first agent binding, in particular at least one (one or two) sequence of up to 50 nucleotides adjacent to said region on CD33 involved in first agent binding, preferably 20, 15, 10, 9, 8, 7, 6 or 5 nucleotides adjacent to said agent binding site.

CRISPR system involves two or more components, Cas protein (CRISPR-associated protein) and a guide RNA. The guide RNA can be a single guide RNA or a dual guide RNA. Cas protein is a DNA endonuclease that uses guide RNA sequence as a guide to recognize and generate double-strand cleavage in DNA that is complementary to the target sequence. Cas systems that generate single strand breaks require only one nuclease domain. Cas systems that generate double strand breaks require two nuclease domains. Cas protein may comprise two active cutting sites, such as HNH nuclease domain and RuvC-like nuclease domain.

By Cas protein is also meant an engineered endonuclease, homologue or orthologue of Cas 9 which is capable of cleaving target nucleic acid sequence. In particular embodiments, Cas protein may induce a cleavage in the nucleic acid target sequence which can correspond to either a double-stranded break or a single-stranded break. Cas protein variant may be a Cas endonuclease that does not naturally exist in nature and that is obtained by protein engineering or by random mutagenesis. The Cas protein can be one type of the Cas proteins known in the art. Non-limiting examples of Cas proteins include Cas1, Cas1B, Cas2, Cas3, Cas4, Cas5, Cas6, Cas7, Cas8, Cas9 (also known as CsnI and CsxI2), SaCas9, Cas12, Cas12a (Cpf1), CasIO, CsyI, Csy2, Csy3, CseI, Cse2, CscI, Csc2, Csa5, Csn2, Csm2, Csm3, Csm4, Csm5, Cmrl, Cmr3, Cmr4, Cmr5, Cnrr6, CsbI, Csb2, Csb3, CsxI7, CsxM, Csx IO, Cs I6, CsaX, Csx3, Cs I, Csx15, CsfI, Csf2, CsO, Csf4, homologs, orthologs thereof, or modified versions thereof. Preferably Cas protein is Streptococcus pyogenes Cas 9 protein.

Cas is contacted with a guide RNA (gRNA) designed to comprise a complementary sequence to the target sequence to specifically induce DNA cleavage within said target sequence, in particular according to the present disclosure a complementary sequence of a part of the target sequence encoding the surface protein region recognized by agent as described above.

As used herein, a “guide RNA”, “gRNA”, “sgRNA” or “single guide RNA” refers to a nucleic acid that promotes the specific targeting or homing of a gRNA/Cas complex to a target nucleic acid.

In particular, gRNA refers to RNA that comprises a transactivating crRNA (tracrRNA) and a crRNA. Preferably, said guide RNA corresponds to a crRNA and tracrRNA which can be used separately or fused together to generate a single guide RNA. The complementary sequence pairing with the target sequence recruits Cas to bind and cleave the DNA at the target sequence.

According to the present disclosure, crRNA is engineered to comprise a complementary sequence to a part of a target sequence as described above encoding surface protein region recognized by agent, such that it is capable of targeting said region. In a preferred embodiment sgRNA is used to target the binding site of the said binding agent. In another preferred embodiment, the guide RNA contains chemically modifications known to the person skilled in the art.

In a particular embodiment, the crRNA comprises a sequence of 5 to 50 nucleotides, preferably 15 to 30 nucleotides, more preferably 20 nucleotides which is complementary to the target sequence. As used herein, the terms “complementary sequence” refers to the sequence part of a polynucleotide (e.g., part of crRNA or tracRNA) that can hybridize to another part of polynucleotides under standard low stringent conditions. Preferentially, the sequences are complementary to each other pursuant to the complementarity between two nucleic acid strands relying on Watson-Crick base pairing between the strands, i.e., the inherent base pairing between adenine and thymine (A-T) nucleotides and guanine and cytosine (G-C) nucleotides. Said gRNA can be designed by any methods known by one of skill in the art in view of the present disclosure.

According to the present disclosure said target sequence encodes surface protein region on CD33 involved in first agent binding, preferably located in the extracellular portion of CD33, more preferably in an extracellular loop in comparison to said second isoform, again more preferably comprising amino acid residues involved in agent binding.

In a preferred embodiment, when surface protein is CD33, said target sequence encodes a CD33 region involved in binding of a first agent, such as anti-CD33 agent binding as disclosed above. Preferably said target sequence encodes at least one residue in position N20, F21, W22, Q24, F43, F44, H45, P48, Y49 and/or Y50 of SEQ ID NO: 1.

The DNA strand break that is introduced by the nuclease according to the disclosure can result in mutation of the DNA at the cleavage site via non-homologous end joining (NHEJ) which often results in small insertions and/or deletions or replacement of the DNA surrounding the cleavage site via homology-directed repair (HDR).

In a preferred embodiment, said polymorphism within nucleic acid encoding the isoform of CD33 is induced via HDR repair following the DNA cleavage and the introduction of an exogeneous nucleotide sequence, named herein HDR template.

HDR template comprises a first and a second portion of sequence which are homologous to regions 5′ and 3′ of the target sequence, respectively and a middle sequence portion comprising polymorphism. Following cleavage of the target sequence, a homologous recombination event is achieved between the genome containing the target sequence and the HDR template and the genomic sequence containing the target sequence is replaced by the exogeneous sequence.

Preferably, homologous sequences of at least 20 bp, preferably more than 30 bp, more preferably more than 50 bp and most preferably less than 200 bp are used. Homologous sequences may be dsDNA or ssDNA. Preferably the homologous sequences are ds DNA. Indeed, shared DNA homologies are located in regions flanking upstream and downstream the site of the break and the exogeneous sequence to be introduced should be located between the two arms. The flanking sequences may be symmetrical or asymmetrical. Both strands of the target nucleic acid, i.e., the plus strand or the minus strand, may be targeted. Optionally, a PAM sequence may be used, which may be silenced to improve HDR.

In a preferred embodiment, the cell according to the present disclosure is genetically engineered by introducing into said cell said site-specific nuclease which targets the sequence encoding the region on CD33 recognized by said first agent as described above and a HDR template.

In another particular embodiment, said gene editing enzyme is a DNA base editor as described in Komor et al., Nature 533, 420-424, and in Rees H A, Liu D R. Nat Rev Genet. 2018; 19: 770-788, or a prime editor as described in Anzalone et al. Nature, 2019, 576: 149-157, Matsoukas et al., Front Genet. (2020) 11: 528, Chen et al. Cell (2021) 184: 5635-52, Koblan et al, Nat Biotechnol (2021) 39: 1414-25 and Kantor A. et al. Int. J. Mol. Sci. 2020, 21(6240). Base editor or prime editor can be used to introduce mutations at specific sites in the target sequence.

According to the present disclosure, the base editor or prime editor generates a mutation within the target sequence by sequence-specific targeting of the sequence encoding the region on CD33 involved in first agent binding.

In particular, said base editor or prime editor are CRISPR base or prime editors. Said CRISPR base or prime editor may comprise as catalytically inactive sequence specific nuclease a dead Cas protein (dCas). It may also comprise Cas9 with a mutated nuclease domain. dCas refers to a modified Cas nuclease which lacks endonucleolytic activity. Nuclease activity can be inhibited or prevented in dCas proteins by one or more mutations and/or one or more deletions in the HNH and/or RuvC-like catalytic domains of the Cas protein. The resulting dCas protein lacks nuclease activity but bind to a guide RNA (gRNA)-DNA complex with high specificity and efficiency to specific target sequence. In particular embodiment, said dCas may be a Cas nickase wherein one catalytic domain of the Cas is inhibited or prevented.

Said base editor is complexed with a guide RNA (gRNA) designed to comprise a complementary sequence of the target nucleic acid sequence to specifically bind said target sequence as described above.

Said gRNA can be designed by any methods known by one of skill in the art in view of the present disclosure. In a particular embodiment, said gRNA may target the sequence encoding the region on CD33 recognized by said first agent as described above.

As non-limiting examples said base editor is a nucleotide deaminase domain fused to a dead Cas protein, in particular Cas nickase. Said nucleotide deaminase may be an adenosine deaminase or cytidine deaminase. Said nucleotide deaminase may be natural or engineered deaminase.

In a particular embodiment, said base editor may be as non-limiting examples selected from the group consisting of: BE1, BE2, BE3, BE4, HF-BE3, Sa-BE3, Sa-BE4, BE4-Gam, saBE4-Gam, YE1-BE3, EE-BE3, YE2-BE3, YEE-BE3, VQR-BE3, VRER-BE3, SaKKH-BE3, cas12a-BE, Target-AID, Target-AID-NG, xBE3, eA3A-BE3, A3A-BE3, BE-PLUS, TAM, CRIPS-X, ABE7.9, ABE7.10, ABE7.10* xABE, ABESa, ABEmax, ABE8e, VQR-ABE, VRER-ABE and SaKKH-ABE.

Said prime editor consists of a fusion of a catalytically inactive sequence specific nuclease as described above, particularly a Cas nickase and a catalytically active engineered reverse transcriptase (RT) enzyme. Said fusion protein is used in combination with a prime editing guide RNA (pegRNA) which contains the complementary sequence to the target sequence as described above, particularly when surface protein is CD33 comprises one of the sequences described herein and also an additional sequence comprising a sequence that binds to the primer binding site region on the DNA. In particular embodiment, said reverse transcriptase enzyme is a Maloney murine leukemia virus RT enzyme and variants thereof. Said prime editor may be as non-limiting examples selected from the group consisting of: PE1, PE2, PE3 and PE3b, or any of the prime editors described in Chen et al. Cell (2021) 184: 5635-52 or Koblan et al, Nat Biotechnol (2021) 39: 1414-25.

### Anti-CD33 Agents

Several anti-CD33 moieties are known in the art, some of which are currently in development. WM53 (BioLegend; #303402) and 2337A (R&D Systems; #MAB11373-100) are commercially available research-grade monoclonal antibodies. Other anti-CD33 moieties include gemtuzumab ozogamicin (Mylotarg), an antibody-drug which was developed by Pfizer/Wyeth-Ayerst, vadastuximab talirine, an antibody-drug conjugate developed by Seagen Inc, lintuzumab a humanized antibody developed by Seagen Inc., BI-836858, a fully human IgG1 anti-CD33 monoclonal antibody developed by Boehringer Ingelheim, AMG330, a bispecific T cell engager with specificity for CD33 and CD3 which is developed by Amgen, and IMG779, an antibody-drug conjugate developed by ImmunoGen Inc. VCAR33 is a CD33-targeted CAR-T cell product developed by VOR Therapeutics. These and other anti-CD33 moieties may be used in the context of the present disclosure. Several anti-CD33 antibodies were also generated in the present disclosure, in full length antibody format, as well as in Fab format. Details are provided in Example 1.

In a particular embodiment, said depleting agent which binds to said second isoform of CD33 and does not bind or binds substantially weaker to said first isoform of CD33 as described above binds specifically to an epitope including the amino acids N20, F21, W22, Q24, F43, F44, H45, P48, Y49 and/or Y50 of SEQ ID NO: 1. In certain preferred embodiments said depleting agents binds specifically to an epitope including the amino acids N20, F21, W22 and/or Y50 of SEQ ID NO: 1. In other preferred embodiments said depleting agents binds specifically to an epitope including the amino acids N20, F21 and W22 of SEQ ID NO: 1. In other preferred embodiments said depleting agents binds specifically to an epitope including the amino acid Y50 of SEQ ID NO: 1.

In a preferred embodiment, said anti-CD33 agent comprises an antigen binding region comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8 and VLCDR3 is SEQ ID NO: 9.

In a preferred embodiment, said anti-CD33 agent comprises an antigen binding region derived from and retaining the binding specificity of an antibody binding comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8 and VLCDR3 is SEQ ID NO: 9.

In a preferred embodiment, said anti-CD33 agent comprises an antigen binding region competing with an antibody binding comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8 and VLCDR3 is SEQ ID NO: 9.

In another preferred embodiment, said anti-CD33 agent comprises an antigen binding region comprising:
a) an antibody heavy chain variable domain (VH) comprising the variable heavy chain of SEQ ID NO: 2; and
b) an antibody light chain variable domain (VL) comprising variable light chain of SEQ ID NO: 3.

In a preferred embodiment, said anti-CD33 agent comprises an antigen binding region comprising:
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8 and VLCDR3 is SEQ ID NO: 24.

In a preferred embodiment, said anti-CD33 agent comprises an antigen binding region derived from and retaining the binding specificity of an antibody binding comprising:
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8 and VLCDR3 is SEQ ID NO: 24.

In a preferred embodiment, said anti-CD33 agent comprises an antigen binding region competing with an antibody binding region comprising:
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8 and VLCDR3 is SEQ ID NO: 24.

In another preferred embodiment, said anti-CD33 agent comprises an antigen binding region comprising:
a) an antibody heavy chain variable domain (VH) comprising the variable heavy chain of SEQ ID NO: 18; and
b) an antibody light chain variable domain (VL) comprising variable light chain of SEQ ID NO: 19.

In a preferred embodiment, said anti-CD33 agent comprises an antigen binding region comprising:
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31 and VLCDR3 is SEQ ID NO: 32.

In a preferred embodiment, said anti-CD33 agent comprises an antigen binding region derived from and retaining the binding specificity of an antibody binding comprising:
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31 and VLCDR3 is SEQ ID NO: 32.

In a preferred embodiment, said anti-CD33 agent comprises an antigen binding region competing with an antibody binding region comprising:
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31 and VLCDR3 is SEQ ID NO: 32.

In another preferred embodiment, said anti-CD33 agent comprises an antigen binding region comprising:
a) an antibody heavy chain variable domain (VH) comprising the variable heavy chain of SEQ ID NO: 25; and
b) an antibody light chain variable domain (VL) comprising variable light chain of SEQ ID NO: 26.

In another preferred embodiment, said anti-CD33 agent is an antibody selected from Refmab #1, Refmab #2, Refmab #3, Refmab #4, Refmab #5, Refmab #6, Rafmab #7 and Refmab #8.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in an amino acid selected from N20, F21 and/or W22 of SEQ ID NO: 1.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in amino acid N20 of SEQ ID NO: 1, and wherein said mutation is N20K.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21S.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in an amino acid selected from N20, F21 and/or W22 of SEQ ID NO: 1.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in amino acid N20 of SEQ ID NO: 1, and wherein said mutation is N20K.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21S.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in an amino acid selected from N20, F21 W22 and/or Q24 of SEQ ID NO: 1.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid N20 of SEQ ID NO: 1, and wherein said mutation is N20G.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21L.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21S.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in an amino acid selected from N20, F21 W22 and/or Q24 of SEQ ID NO: 1.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid N20 of SEQ ID NO: 1, and wherein said mutation is N20G.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21L.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24,
   wherein said depleting agent binds to one isoform of CD33 but not, or substantially weaker, to a second isoform of CD33, wherein one of said isoform is wild type CD33 and the other isoform has a mutation in amino acid F21 of SEQ ID NO: 1, and wherein said mutation is F21S.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1, and wherein said mutation is Y50D.

In certain preferred embodiments, said depleting agent comprises an antigen binding region comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1, and wherein said mutation is Y50R.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1, and wherein said mutation is Y50D.

In certain preferred embodiments, said depleting agent comprises an antigen binding region derived from and retaining the binding specificity of an antibody binding comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32,
   wherein said depleting agent binds to one isoform of CD33 but not, or substantially weaker, to a second isoform of CD33, wherein one of said isoform is wild type CD33 and the other isoform has a mutation in amino acid Y50 of SEQ ID NO: 1, and wherein said mutation is Y50R.

It is further contemplated that the antigen-binding region of the anti-CD33 antibody may be further screened or optimized for their binding properties as above defined. In particular, it is contemplated that said antigen binding region thereof may have 1, 2, 3, 4, 5, 6, or more alterations in the amino acid sequence of 1, 2, 3, 4, 5, or 6 CDRs of monoclonal antibodies provided herein. It is contemplated that the amino acid in position 1, 2, 3, 4, 5, 6, 7, 8, 9, or 10 of CDR1, CDR2, CDR3, CDR4, CDR5, or CDR6 of the VJ or VDJ region of the light or heavy variable region of antigen binding region may have an insertion, deletion, or substitution with a conserved or non-conserved amino acid. Such amino acids that can either be substituted or constitute the substitution are disclosed above.

In some embodiments, the amino acid differences are conservative substitutions, i.e., substitutions of one amino acid with another having similar chemical or physical properties (size, charge or polarity), which substitution generally does not adversely affect the biochemical, biophysical and/or biological properties of the antibody. In particular, the substitution does not disrupt the interaction of the antibody with the CD33 antigen. Said conservative substitution(s) are advantageously chosen within one of the following five groups: Group 1-small aliphatic, non-polar or slightly polar residues (A, S, T, P, G); Group 2-polar, negatively charged residues and their amides (D, N, E, Q); Group 3-polar, positively charged residues (H, R, K); Group 4-large aliphatic, nonpolar residues (M, L, I, V, C); and Group 5-large, aromatic residues (F, Y, W).

In a more particular embodiment, said first antigen-binding region comprises a heavy chain variable domain comprising or consisting of any one of amino acid sequences selected from SEQ ID NO: 2, 10, 18, 25, 33 and 41 and/or a light chain variable domain comprising or consisting of any one of amino acid sequences selected from SEQ ID NO: 3, 11, 19, 26, 34 and 42.

Said first antigen binding region thereof with amino acid sequences having at least 90%, for example, at least 95%, 96%, 97%, 98%, or 99% identity to any one of the above defined amino acid sequences are also part of the present disclosure, typically first antigen binding region have at least equal or higher binding activities than said first antigen binding region consisting of heavy chain consisting of any one of amino acid sequences selected from SEQ ID NO: 2, 10, 18, 25, 33 and 41 and/or a light chain variable domain comprising or consisting of any one of amino acid sequences selected from SEQ ID NO: 3, 11, 19, 26, 34 and 42.

In a particular embodiment, said anti-CD33 agent can be a bispecific CD33 antibody, comprising at least one first binding specificity for CD33, for example, one antigen-binding region of anti-CD33 as described herein and a second binding specificity for a second target epitope or target antigen.

According to the present disclosure, said anti-CD33 agent can be an immune cell harboring an antigen receptor targeting CD33, such as a CAR targeting CD33, said antigen receptor comprising an antigen binding region as described herein.

In specific embodiments, said immune cell (e.g. T cell) harboring a CAR targeting CD33 recognizes a second isoform of CD33 as expressed in a patient in need thereof, and does not recognize a first isoform of CD33. In particular said immune cell may bind specifically to an epitope including the amino acids N20, F21, W22, Q24, F43, F44, H45, P48, Y49 and/or Y50 of SEQ ID NO: 1. In other particular said immune cell may bind specifically to an epitope including the amino acids N20, F21, W22 and/or Y50 of SEQ ID NO: 1.

In specific embodiments, said anti-CD33 agent can be an immune cell (e.g. T cell) harboring a CAR, said CAR comprising or derived from an antigen-binding region comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9.

In a more particular embodiment, said anti-CD33 agent can be an immune cell (e.g. T cell) harboring a CAR comprising said first antigen-binding region e.g. scFv comprising a heavy chain variable domain comprising or consisting the amino acid sequence of SEQ ID NO: 2 and/or a light chain variable domain comprising or consisting the amino acid sequence of SEQ ID NO: 3.

In specific embodiments, said anti-CD33 agent can be an immune cell (e.g. T cell) harboring a CAR, said CAR comprising or derived from an antigen-binding region comprising
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8 and VLCDR3 is SEQ ID NO: 24.

In a more particular embodiment, said anti-CD33 agent can be an immune cell (e.g. T cell) harboring a CAR comprising said first antigen-binding region e.g. scFv comprising a heavy chain variable domain comprising or consisting of an amino acid sequence of SEQ ID NO: 18 and a light chain variable domain comprising or consisting of the amino acid sequence of SEQ ID NO: 19.

In specific embodiments, said anti-CD33 agent can be an immune cell (e.g. T cell) harboring a CAR, said CAR comprising or derived from an antigen-binding region comprising
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31 and VLCDR3 is SEQ ID NO: 32.

In a more particular embodiment, said anti-CD33 agent can be an immune cell (e.g. T cell) harboring a CAR comprising said first antigen-binding region e.g. scFv comprising a heavy chain variable domain comprising or consisting of an amino acid sequence of SEQ ID NO: 25 and a light chain variable domain comprising or consisting of the amino acid sequence of SEQ ID NO: 26.

In another preferred embodiment said anti-CD33 agent can be an immune cell harboring a CAR targeting a specific isoform of CD33 as described in the examples.

In particular, the disclosure also relates to depleting anti-CD33 agents as disclosed above (for example CAR cell composition or antibodies) comprising a first or a second antigen binding region for use in selectively depleting the host cells or transferred cells respectively, in a subject in need thereof.

### Cells Expressing a First Isoform of CD33

The present disclosure relates to a mammalian cell, preferably a hematopoietic cell, or a population of cells expressing a first isoform of CD33 wherein said cell or population of cells express a first isoform of CD33, wherein said first isoform is not recognized by the depleting agent comprising a first antigen binding region as described herein.

The present disclosure also relates to a mammalian cell, preferably a hematopoietic cell, or a population of cells expressing a first isoform of CD33 wherein said cell or population of cells express a first isoform of CD33 comprising at least one polymorphic allele in the nucleic acid encoding said first isoform, and wherein said first isoform is not recognized by the depleting agent comprising a first antigen binding region as described herein.

The cell expressing said first isoform of CD33 which is not recognized by the depleting agent comprising a first antigen binding region as described herein may not necessarily comprise said polymorphism or genetically engineered allele in the genomic DNA.

Said first isoform of CD33 may also be transiently expressed in said cell by any methodology known to the person skilled in the art.

Said first isoform of CD33 may also be generated in said cell by in vivo editing by any appropriate means known to the person skilled in the art.

Said cell or population of cells are particularly useful in medical treatment in a patient expressing a second isoform of CD33.

In a particular embodiment, said cells (e.g. hematopoietic stem cell) encoding or expressing said first isoform of CD33 not recognized by a depleting agent (e.g. hematopoietic cells) are particularly useful in medical treatment to restore normal hematopoiesis after immunotherapy, such as adoptive cell transfer in a patient expressing said second isoform, in particular wherein the treatment comprises administering a therapeutically efficient amount of said hematopoietic cells expressing said first isoform of CD33 in combination with a therapeutically efficient amount of a depleting agent targeting said second isoform of CD33. In particular, said hematopoietic cells, preferably hematopoietic stem cells are administered subsequently to said depleting agent. In another particular embodiment, said hematopoietic cells, preferably hematopoietic stem cells can be administered before or concurrently to said depleting agent.

In another particular embodiment, said cells expressing said first isoform of CD33 specifically recognized by depleting agent which does not bind or binds substantially weaker second isoform of CD33 are particularly useful in medical treatment in a patient expressing said second isoform of CD33, in particular to avoid severe side-effect related to transplanted cells carrying the first isoform (safety switch), wherein the treatment comprises administering a therapeutically efficient amount of a depleting agent targeting said first isoform of CD33. In particular, said hematopoietic cells, preferably immune cells harboring a CAR are administered prior to said depleting agent.

As used herein, the term cell relates to mammalian cells, preferably human cells.

In a particular embodiment, said cells are hematopoietic cells. Hematopoietic cells comprise immune cells including lymphocytes, such as B cells and T cells, natural killer cells, myeloid cells, such as monocytes, macrophages, eosinophils, mast cells, basophils, granulocytes, dendritic cells (DC) and plasmacytoid dendritic cells (pDCs).

In certain embodiments, said immune cells are T cells. In another preferred embodiment, said immune cells are primary T cells. As used herein, the term “T cell” includes cells bearing a T cell receptor (TCR) or a cell derived from a T cell bearing a TCR. T-cells according to the disclosure can be selected from the group consisting of inflammatory T-lymphocytes, cytotoxic T-lymphocytes, regulatory T-lymphocytes, memory T-lymphocytes, tumor infiltrating lymphocytes or helper T-lymphocytes included both type 1 and 2 helper T cells and Th17 helper cells. In another embodiment, said cell can be derived from the group consisting of CD4+T-lymphocytes and CD8+T-lymphocytes or non-classical T cells such as MR1 restricted T cells, MAIT cells, NKT cells, gamma delta T cells or innate-like T cells.

T-cells can be obtained from a number of non-limiting sources, including peripheral blood mononuclear cells, bone marrow, lymph node tissue, cord blood, thymus tissue, tissue from a site of infection, ascites, pleural effusion, spleen tissue, and tumors. In certain embodiments, T-cells can be obtained from a unit of blood collected from a subject using any number of techniques known to the skilled person. Alternatively, T cells can be differentiated from iPS cells.

In another preferred embodiment, said hematopoietic cells are hematopoietic stem cells. The stem cells can be adult stem cells, embryonic stem cells, more particularly non-human stem cells, cord blood stem cells, progenitor cells, bone marrow stem cells, induced pluripotent stem cells, totipotent stem cells or hematopoietic stem cells. Representative human stem cells are CD34+ cells. Hematopoietic stem cells can be differentiated from iPS cells or can be harvested from umbilical cord blood, from bone marrow or from mobilized or not mobilized peripheral blood.

In certain embodiments, the cell is an allogeneic cell which refers to a cell derived from a donor that presents with an HLA genotype that is identical, similar or different to the HLA genotype of the person receiving the cell. The donor may be a related or unrelated person. In certain embodiments, the cell is an autologous cell which refers to a cell derived from the same person that is receiving the cell.

Said cells may originate from a healthy donor or from a patient, in particular from a patient diagnosed with cancer, genetic disease or an auto-immune disease or from a patient diagnosed with an infection. Hematopoietic cells can be extracted from blood, bone marrow or derived from stem cells. HSC's can for example be derived from iPS (induced pluripotent stem cells.

A person skilled in the art will choose the more appropriate cells according to the patient or subject to be transplanted.

The disclosure further relates to a composition of cells or a population of cells for use in the therapy as disclosed herein.

In certain embodiments, the present disclosure relates to a mammalian cell or a population of cells expressing a first isoform of CD33 for use in a medical treatment in a patient in need thereof, wherein said patient has cells expressing a second isoform of CD33, wherein said cell expressing said first isoform comprises genomic DNA with at least one polymorphism or genetically engineered allele, wherein said polymorphism or genetically engineered allele is not present in the genome of the patient having cells expressing said second isoform of CD33, and wherein said polymorphic or genetically engineered allele is characterized by at least one substitution of an amino acid in position N20, F21, W22, Q24, F43, F44, H45, P48, Y49 and/or Y50 of SEQ ID NO: 1. In certain preferred embodiments, said polymorphic or genetically engineered allele is characterized by at least one substitution of an amino acid in position N20, F21, W22 and/or Y50 of SEQ ID NO: 1.

In other preferred embodiments, said polymorphic or genetically engineered allele is characterized by at least one substitution of an amino acid in position N20, F21 and W22 of SEQ ID NO: 1. In yet other preferred embodiments, said polymorphic or genetically engineered allele is characterized by a substitution of amino acid in position Y50 of SEQ ID NO: 1.

### CAR

For use in adoptive cell transfer therapy, said cell expressing first isoform of CD33 according to the present disclosure may be modified to display desired specificities and enhanced functionalities. In a particular embodiment, said cell may express a recombinant antigen binding region, also named antigen receptor on its cell surface as described above. In a particular embodiment, said recombinant antigen receptor is a chimeric antigen receptor (CAR). According to the present disclosure, said immune cell expressing a first isoform of CD33 and a CAR can be specifically depleted by the administration of a therapeutically efficient amount of an agent which comprises a second antigen binding region which specifically binds to said first isoform of CD33 but not to the second isoform of CD33, thereby avoiding eventual severe side effects due to transplantation of said immune cells.

In a particular embodiment, the immune cell is redirected against a cancer antigen. By “cancer antigen” is meant any antigen (i.e., a molecule capable of inducing an immune response) which is associated with cancer. An antigen as defined herein may be any type of molecule which induces an immune response, e.g., it may be a polysaccharide or a lipid, but most preferably it is a peptide (or protein). Human cancer antigens may be human or human-derived. A cancer antigen may be a tumor-specific antigen, by which is meant an antigen which is not found in healthy cells. Tumor-specific antigens generally result from mutations, in particular frame-shift mutations which generate a wholly new amino acid sequence not found in the healthy human proteome.

Cancer antigens also include tumor-associated antigens, which are antigens whose expression or production is associated with, but not limited to, tumor cells. Examples of tumor-associated antigens include for instance Her2, prostate stem cell antigen (PSCA), alpha-fetoprotein (AFP), carcinoembryonic antigen (CEA), cancer antigen-125 (CA-125), CA19-9, calretinin, MUC-1, epithelial membrane protein (EMA), epithelial tumor antigen (ETA), tyrosinase, melanoma-associated antigen (MAGE), CD34, CD45, CD99, CD117, CD123, chromogranin, cytokeratin, desmin, glial fibrillary acidic protein (GFAP), gross cystic disease fluid protein (GCDFP-15), HMB-45 antigen, protein melan-A (melanoma antigen recognized by T lymphocytes; MART-1), myo-DI, muscle-specific actin, neurofilament, neuron-specific enolase (NSE), placental alkaline phosphatase, synaptophysin, thyroglobulin, thyroid transcription factor-1, the dimeric form of the pyruvate kinase isoenzyme type M2 (tumor M2-PK), CD 19, CD22, CD45, CD123, CD27, CD30, CD70, GD2 (ganglioside G2), EGFRvIII (epidermal growth factor variant III), sperm protein 17 (Spl7), mesothelin, PAP (prostatic acid phosphatase), prostein, TARP (T cell receptor gamma alternate reading frame protein), Trp-p8, STEAP1 (six-transmembrane epithelial antigen of the prostate 1), an abnormal ras protein, or an abnormal p53 protein. In another specific embodiment, said tumor-associated antigen or tumor-specific antigen is integrin αvβ3 (CD61), galactin, K-Ras (V-Ki-ras2 Kirsten rat sarcoma viral oncogene), or Ral-B.

In a particular embodiment, for use in adoptive cell transfer therapy, preferably for the treatment of malignant hematopoietic disease such as acute myeloid leukemia (AML) or B-acute lymphoblastic leukemia (B-ALL), the immune cell according to the present disclosure expresses a recombinant antigen binding region such as a CAR targeting CD33. Said cell expressing the first isoform and expressing the CAR (e.g. CAR-CD33) can be further specifically depleted by administering a depleting agent comprising a second antigen-binding region which binds specifically to the first isoform of CD33, but does not bind or binds substantially weaker to the second isoform of CD33, thereby avoiding eventual severe side effects such as graft-versus-host disease due to the transplantation.

In specific embodiments, said immune cell expressing the first isoform harbors a CAR targeting CD33, said CAR comprising an antigen-binding region, e.g. scFv, comprising an antigen-binding region which binds specifically to an epitope of CD33 located within the N-terminal domain, or within the polypeptide including the amino acids N20, F21, W22, Q24, F43, F44, H45, P48, Y49 and/or Y50 of SEQ ID NO: 1.

In particular, said immune cell expressing first isoform harbors a CAR targeting CD33 comprising an antigen-binding region, e.g. scFv, comprising or derived from
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9,
   more preferably comprising an antigen-binding region comprising a heavy chain variable domain comprising or consisting of the amino acid sequence of SEQ ID NO: 2 and/or a light chain variable domain comprising or consisting of the amino acid sequence of SEQ ID NO: 3.

In particular, said immune cell expressing first isoform harbors a CAR targeting CD33 comprising an antigen-binding region, e.g. scFv, comprising or derived from
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8 and VLCDR3 is SEQ ID NO: 24,
   more preferably comprising an antigen-binding region comprising a heavy chain variable domain comprising or consisting of the amino acid sequence of SEQ ID NO: 18 and/or a light chain variable domain comprising or consisting of the amino acid sequence of SEQ ID NO: 19.

In particular, said immune cell expressing first isoform harbors a CAR targeting CD33 comprising an antigen-binding region, e.g. scFv, comprising or derived from
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31 and VLCDR3 is SEQ ID NO: 32,
   more preferably comprising an antigen-binding region comprising a heavy chain variable domain comprising or consisting of the amino acid sequence of SEQ ID NO: 25 and/or a light chain variable domain comprising or consisting of the amino acid sequence of SEQ ID NO: 26.

### In Vitro Method for Preparing Cell Expressing First Isoform

The cell expressing the first isoform of CD33 according to the present disclosure can be genetically engineered by introducing into said cell a nucleic acid construct (e.g., mRNA) encoding at least one gene editing enzyme or ribonucleoprotein complex comprising gene editing enzyme and/or HDR template as described above. Alternatively, the gene editing system is transduced into said cells via a viral system, such as an adenoviral system. Said cell can also be genetically engineered by further introducing into said cell a nucleic acid construct encoding a CAR as described above. In particular, said method is an ex vivo method performed on a culture of cells.

The term “nucleic acid construct” as used herein refers to a nucleic acid molecule resulting from the use of recombinant DNA technology. A nucleic acid construct is a nucleic acid molecule, either single- or double-stranded, which has been modified to contain segments of nucleic acid sequences, which are combined and juxtaposed in a manner, which would not otherwise exist in nature. A nucleic acid construct usually is a “vector”, i.e., a nucleic acid molecule which is used to deliver exogenously created DNA into a host cell.

Preferably, the nucleic acid construct comprises said gene editing enzyme, HDR template and/or CAR, operably linked to one or more control sequences. Said control sequences may be a ubiquitous, tissue-specific or inducible promoter which is functional in cells of target organs (i.e., hematopoietic cell). Such sequences which are well-known in the art include in particular a promoter, and further regulatory sequences capable of further controlling the expression of a transgene, such as without limitation, enhancer, terminator, intron, silencer.

The nucleic acid construct as described above may be contained in an expression vector. The vector may be an autonomously replicating vector, i.e., a vector that exists as an extra-chromosomal entity, the replication of which is independent of chromosomal replication, e.g., a plasmid, an extra-chromosomal element, a mini-chromosome, or an artificial chromosome. The vector may contain any means for assuring self-replication. Alternatively, the vector may be one that, when introduced into the host cell, is integrated into the genome and replicated together with the chromosome(s) into which it has been integrated.

Examples of appropriate vectors include, but are not limited to, recombinant integrating or non-integrating viral vectors and vectors derived from recombinant bacteriophage DNA, plasmid DNA or cosmid DNA. Preferably, the vector is a recombinant integrating or non-integrating viral vector. Examples of recombinant viral vectors include, but not limited to, vectors derived from herpes virus, retroviruses, lentivirus, vaccinia viruses, adenoviruses, adeno-associated viruses or bovine papilloma virus.

The present disclosure relates to a method for expressing a first isoform of a cell surface protein in a cell by introducing into said cell a nucleic acid construct (e.g. mRNA) encoding the gene editing enzyme or ribonucleoprotein complex comprising gene editing enzyme and/or HDR template as described above. Said method may further comprise a step of introducing into said cell a nucleic acid construct encoding a CAR. Said method involves introducing gene editing enzyme such as Cas protein, base editor or prime editor and guide RNA (crRNA, tracrRNA, or fusion guide RNA or pegRNA) into a cell. In particular, said gene editing enzyme is CRISPR/Cas gene editing enzyme as described above. In a more particular embodiment, said gene editing enzyme is a site-specific nuclease, more preferably CRISPR/Cas nuclease comprising a guide RNA and Cas protein, wherein said guide RNA in combination with Cas protein cleaves and induces cleavage within said target sequence comprising a nucleic acid encoding surface protein region involved in agent binding as described above.

Said Cas nuclease may be a high-fidelity Cas nuclease such as a high fidelity Cas9 nuclease.

Said gene editing enzyme, preferably guide RNA and/or Cas protein, base editor or prime editor as described above may be synthesized in situ in the cell as a result of the introduction of nucleic acid construct, preferably expression vector encoding said gene editing enzyme such as guide RNA and/or Cas protein, base editor or prime editor as described above into the cell. Alternatively, said gene editing enzyme such as guide RNA and/or Cas protein, base editor or prime editor may be produced outside the cell and then introduced thereto.

Said nucleic acid construct or expression vector can be introduced into cell by any methods known in the art and include, as non-limiting examples, stable transduction methods in which the nucleic acid construct or expression vector is integrated into the cell genome, transient transfection methods in which the nucleic acid construct or expression vector is not integrated into the genome of the cell and virus-mediated methods. For example, transient transformation methods include for example microinjection, electroporation, cell squeezing, particle bombardment or in vivo targeting approaches.

### In Vivo Editing

The cell expressing the first isoform of CD33 according to the present disclosure may also be edited in vivo. Various technologies exist that enable therapeutic in vivo gene editing, including viral vectors, lipid nanoparticles and virus-like particles (see for example Cell (2022) 185: 2806-27). The molecular machinery to convert CD33 into a first isoform of CD33 which is not recognized by the depleting agent can be accomplished by any of these methods.

In certain embodiments, the present disclosure relates a pharmaceutical composition comprising molecular machinery capable of in vivo editing a gene and a depleting agent,

wherein said molecular machinery capable of in vivo editing a gene comprises all components required to introduce a point mutation of wild type CD33 in a target cell into an isoform of CD33, and
wherein said depleting agent binds to wild type CD33, but not to said isoform of CD33 for use in a medical treatment in a patient in need thereof.

### Pharmaceutical Composition and Therapeutic Use

In a further aspect, the present disclosure also provides a pharmaceutical composition comprising cells or a population of cells expressing a first isoform of CD33 as described above with one or more pharmaceutically or physiologically acceptable carriers, diluents or excipients.

In a particular embodiment, said cell expressing the first isoform of CD33 is a hematopoietic stem cell.

In another embodiment, said cell expressing said first isoform of CD33 is an immune cell, preferably a T-cell, more preferably a primary T cell, bearing a chimeric antigen receptor (CAR), preferably a CAR which targets the second isoform of CD33 expressed by said patient's cells as described above.

The pharmaceutical composition may further comprise a depleting agent comprising a first or second antigen binding region as described above.

The pharmaceutical composition is formulated in a pharmaceutically acceptable carrier according to the route of administration. Preferably, the composition is formulated to be administered by intravenous injection. Pharmaceutical compositions suitable for such administration may comprise the cells expressing first isoform as described above, in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

Optionally, the composition comprising cells expressing first isoform of CD33 may be frozen for storage at any temperature appropriate for storage of the cells. For example, the cells may be frozen at about −20° C., −80° C. or any other appropriate temperature. Cryogenically frozen cells may be stored in appropriate containers and prepared for storage to reduce risk of cell damage and maximize the likelihood that the cells will survive thawing. Alternatively, the cells may also be maintained at room temperature of refrigerated, e.g., at about 4° C.

The present disclosure relates to the cell or population of cells expressing a first isoform od CD33 as described above for use as a medicament, in particular for use in immunotherapy such as adoptive cell transfer therapy in a patient.

According to the present disclosure, said cell or population of cells (e.g., hematopoietic cells) expressing a first isoform of CD33 as described above, is used in a medical treatment in a patient in need thereof, wherein said medical treatment comprises administering a therapeutically efficient amount of cell or population of cells expressing said first isoform of CD33, in combination with a therapeutically efficient amount of a depleting agent (e.g. a CAR cell or antibody) that binds specifically to the second isoform or first isoform of CD33 to specifically depleting the patients or the transplanted cells, respectively.

As used herein, the term “in combination” or “in combination therapy” means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. The delivery can be such that an effect of the first treatment delivered is still detectable when the second is delivered. In one embodiment, a depleting agent that binds to a second isoform or a first isoform of CD33 is administered at a dose and/or dosing schedule described herein, and the cells expressing the first isoform are administered at a dose and/or a dosing schedule described herein. In some embodiments, “in combination with,” is not intended to imply that the depleting agent targeting the second (e.g. CAR cells or antibody recognizing a second isoform of CD33) or the first isoform of CD33 and compositions of cells expressing said first isoform of CD33, must be administered at the same time and/or formulated for delivery together, although these methods of delivery are within the scope of this disclosure. The depleting agent (e.g. CAR cells or antibody targeting a second isoform of CD33) can be administered concurrently with, prior to or subsequent to a dose of the hematopoietic stem cells expressing the first isoform of CD33. In certain embodiments, each agent will be administered at a dose and/or on a time schedule determined for that particular agent.

Adoptive cell transfer therapy according to the disclosure can be used to treat patients diagnosed with cancer, genetic disease, autoimmune disease, infectious disease, a disease requiring a hematopoietic stem cell transplantation (HSCT), the prevention of organ rejection, the tumor conditioning regimen, tumor maintenance treatment, minimal residual disease, the prevention of relapse.

The present disclosure also relates to the use of cells expressing a first isoform of CD33 as described above in the manufacture of a medicament for adoptive transfer cell therapy in a patient.

As used herein, the term “subject”, or “patient” refers to an animal, preferably to a mammal in which an immune response can be elicited including human, pig, chimpanzee, dog, cat, cow, mouse, rabbit or rat. More preferably, the patient is a human, including adult, child and human at the prenatal stage.

As used herein, the term “treatment”, “treat” or “treating” refers to any act intended to ameliorate the health status of patients such as therapy, prevention, prophylaxis, and retardation of the disease. In certain embodiments, such term refers to the amelioration or eradication of a disease or symptoms associated with a disease. In other embodiments, this term refers to minimizing the spread or worsening of the disease resulting from the administration of one or more therapeutic agents to a subject with such a disease.

Cancers that may be treated include tumors that are not vascularized, or not yet substantially vascularized, as well as vascularized tumors. The cancers may comprise non-solid tumors (such as hematological tumors, for example, leukemias and lymphomas including relapses and treatment-related tumors e.g. secondary malignancies after use of cytotoxic therapy and hematopoietic stem cell transplantation (HSCT)) or may comprise solid tumors.

The term “autoimmune disease” as used herein is defined as a disorder that results from an autoimmune response. An autoimmune disease is the result of an inappropriate and excessive response to a self-antigen.

Infectious disease is a disease caused by pathogenic microorganism such as bacteria, viruses, parasites or fungi. In particular embodiments, infections according to the disclosure occur in immunosuppressed patients, such as patients after HSCT or patients who received a solid organ transplantation.

In a preferred embodiment, the present disclosure relates to a cell expressing first isoform of CD33 as described above for use in hematological cancer, preferably leukemia or lymphoproliferative disorders. Said leukemia can be selected from the group consisting of: acute myeloid leukemia (AML), myelodysplastic syndrome (MDS), chronic myeloid leukemia (CML), chronic myelomonocytic leukemia (CMML), polycythemia vera (PV), essential thrombocythemia (ET), primary myelofibrosis (PMF), blastic plasmacytoid dendritic cell neoplasm (BPDCN) and other myeloproliferative neoplasms and preferably MDS or preferably AML.

In a particular embodiment, said cell or population of cells (e.g., hematopoietic cells) expressing a first isoform of CD33, can be used for the treatment of solid tumor, in particular for selective depletion of myeloid cells in solid tumors in a patient, to enable immunotherapy agent such as immune checkpoint inhibitors, CAR T-cells or tumor infiltrating lymphocytes to access to tumors since myeloid cells in tumors can be immunosuppressive. In this situation said cell or population of cells (e.g., hematopoietic cells) expressing a first isoform CD33 as described above, can serve to replenish the hematopoietic system that might be affected by the treatment intended to deplete the myeloid cells in solid tumors.

In another particular embodiment, said cell or population of cells (e.g., hematopoietic cells) expressing a first isoform of CD33 as described above can be used for the treatment of autoimmune disease such as lupus, multiple sclerosis, scleroderma or systemic sclerosis.

The disclosure also relates to depleting agents (for example CAR cell composition or antibodies) comprising a first or a second antigen binding region for use in selectively depleting the host cells or transferred cells respectively, in a subject in need thereof.

### Method for Depleting Specifically Patient Cells and not Transplanted Cells

According to the present disclosure, said cell or population of cells (e.g. hematopoietic cells) expressing a first isoform of CD33 as described above, is used in a medical treatment in a patient in need thereof, wherein said medical treatment comprises administering a therapeutically efficient amount of said cells or population of cells expressing said first isoform of CD33, in combination with a therapeutically efficient amount of a depleting agent (e.g. a CAR cell or antibody) that binds specifically to a second isoform of CD33.

Indeed, during immunotherapy, immunodepleting agent, such as a CAR expressing immune cells directed to CD33, can be administered to a patient to target and kill tumoral cells. However, as tumoral surface proteins are also expressed at the surface of normal hematopoietic cells, this strategy can induce severe side effects to the patients by altering hematopoiesis. To restore hematopoiesis in the patient, hematopoietic cells can be subsequently transplanted into the patient. However, these cells need to be resistant to said agent, i.e., the depleting agent for CD33 expressing cells, in order not to be targeted by it.

Thus, alternatively, according to the present disclosure, the depleting agent comprising a first antigen binding region which binds specifically to a second isoform of CD33 can be administered to ablate specifically patient cells expressing said second isoform of CD33 and not transplanted cells expressing said first isoform of CD33. The selective depletion of patient cells, but not transplanted cells, allows to reconstitute the patient with a healthy hematopoietic system which will no longer be depleted by immunodepleting agent. Thus, according to the present therapeutic use, the patients have a functional immune system rather than go through a prolonged phase of immunodepression. The use of cells according to the present disclosure eliminates infections as a major complication of current HSC transplantation.

In another embodiment, the present disclosure relates to a method for adoptive cell transfer therapy, preferably for hematopoietic stem cell transplantation to restore normal hematopoiesis in a patient having cells expressing a second isoform of CD33 comprising:
(i) administering an effective amount of a cell (e.g. hematopoietic stem cells) expressing a first isoform of CD33 wherein said cell expressing said first isoform of CD33 comprises genomic DNA with at least one polymorphic allele, preferably single nucleotide polymorphism (SNP) allele, or a genetically engineered allele in the nucleic acid encoding said first isoform and wherein said polymorphism is not present in the genome of the patient having cells expressing said second isoform of CD33 or a pharmaceutical composition thereof; and
(ii) administering a therapeutically efficient amount of an agent comprising at least a first antigen-binding region which binds specifically to said second isoform of CD33 and does not bind or binds substantially weaker to said first isoform of CD33 to deplete specifically cells expressing said second isoform of CD33 (patient's cells).

Said cells expressing the first isoform of CD33 or pharmaceutical compositions thereof are administered to a subject in combination with (e.g., before, simultaneously or following) an agent comprising a first antigen binding region as described above.

In a preferred embodiment, the depleting agent (e.g., CAR cells or antibody targeting a second isoform of CD33 is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform of said surface protein (e.g., a first isoform of CD33).

By a “therapeutically efficient amount” or “effective amount” is intended a number of cells, in particular hematopoietic stem cells expressing the first isoform of CD33 as described above administered to a subject that is sufficient to constitute a treatment as defined above, in particular restoration of normal hematopoiesis in a patient.

The administration of the cell or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present disclosure are preferably administered by intravenous injection. The cells or pharmaceutical compositions of the present disclosure may be injected directly into a tumor, lymph node, or site of infection.

The administration of the cells or population of cells can consist of the administration of 10⁴-10⁹ cells per kg body weight, preferably 10⁵ to 10⁷ cells/kg body weight, more preferably 2×10⁶-5×10⁶ cells per kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

In particular, the disclosure also relates to depleting anti-CD33 agents as disclosed above (for example CAR cell composition or antibodies) comprising a first antigen binding region for use in selectively depleting the host cells in a subject in need thereof.

### Method for Depleting Specifically Transplanted Cells and not Patient Cells (Safety Switch).

According to the present disclosure, said cell or population of cells (e.g. hematopoietic cells) expressing a first isoform of CD33 as described above, is used in a medical treatment in a patient in need thereof, wherein said medical treatment comprises administering a therapeutically efficient amount of a cell or a population of cells expressing said first isoform of CD33, in combination with a therapeutically efficient amount of a depleting agent (e.g. a CAR cell or antibody) that binds specifically to said first isoform CD33.

The cell or population of cells, preferably immune cells expressing the first isoform of CD33 of the present disclosure is particularly used in adoptive transfer cell transfer therapy into a patient. Said transplanted cell expressing said first isoform of CD33 can be further depleted in patients by administering a therapeutically efficient amount of a depleting agent comprising a second antigen binding region which specifically binds to the first isoform of CD33 particularly and does not bind or binds substantially weaker to the second isoform of CD33 expressed by patient's cells to avoid eventual severe side effects such as graft-versus-host disease due to the transplantation. In this case, said agent comprising a second antigen-binding region which binds specifically to said first isoform of CD33 (expressed by transplanted cell) is administered to deplete specifically transplanted cells and not patient cells. Selective depletion of the transplanted cells constitutes an important safety feature by providing a “safety switch”.

Graft-versus-host disease (GvHD) relates to a medical complication following the receipt of transplanted tissue from a genetically different person. Immune cells in the donated tissue (the graft) recognize the recipient (the host) as foreign. In certain embodiments, the medical condition is graft-versus-host disease caused by hematopoietic stem cell transplantation or adoptive cell transfer therapy wherein immune cells are transferred into patient.

Said side effects can also occur when transplanted cells, particularly immune cells harboring a CAR have severe side effects such as cytokine release syndrome and/or neurotoxicity. In this case, the transplanted cells expressing the first isoform of CD33 can be eliminated when said cells become malignant or cause any type of unwanted on-target or off-target damage as a safety switch.

The present disclosure relates to a method for adoptive cell transfer therapy in a patient having cells expressing a second isoform of CD33 comprising:
(i) administering an effective amount of a cell expressing a first isoform of CD33 wherein said cell expressing said first isoform of CD33 comprises genomic DNA with at least one polymorphism allele, preferably single nucleotide polymorphism (SNP) allele, or a genetically engineered allele in the nucleic acid encoding said first isoform CD33 and wherein said polymorphism is not present in the genome of the patient having cells expressing said second isoform of CD33 or a pharmaceutical composition thereof; and
(ii) administering a therapeutically efficient amount of an agent comprising at least a second antigen-binding region which binds specifically to said first isoform of CD33 and does not bind or binds substantially weaker to said second isoform of CD33 to deplete specifically cells expressing said first isoform of CD33.

Said cells expressing the first isoform of CD33 or pharmaceutical compositions thereof are administered to a subject in combination with (e.g., before, simultaneously or following) an agent comprising a second antigen binding region as described above.

In a preferred embodiment, the depleting agent (e.g. CAR cells or antibody targeting a second isoform of CD33) is administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks before), or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, 12 weeks, or 16 weeks after) a dose of the hematopoietic stem cells expressing a first isoform of CD33.

The administration of the cells or pharmaceutical composition according to the present disclosure may be carried out in any convenient manner, including injection, transfusion, implantation or transplantation. The compositions described herein may be administered to a patient subcutaneously, intradermal, intratumorally, intranodally, intramedullary, intramuscularly, by intravenous or intralymphatic injection, or intraperitoneally. In another embodiment, the cells or pharmaceutical compositions of the present disclosure are preferably administered by intravenous injection. The cells or pharmaceutical compositions of the present disclosure may be injected directly into a tumor, lymph node, or site of infection.

The administration of the cells or population of cells can consist of the administration of 10⁴-10⁹ cells per kg body weight, preferably 10⁵ to 10⁷ cells/kg body weight including all integer values of cell numbers within those ranges. The dosage administrated will be dependent upon the age, health and weight of the recipient, kind of concurrent treatment, if any, frequency of treatment and the nature of the effect desired. The cells or population of cells can be administrated in one or more doses. Timing of administration is within the judgment of managing physician and depends on the clinical condition of the subject. The cells or population of cells may be obtained from any source, such as a blood bank or a donor. While individual needs vary, determination of optimal ranges of effective amounts of a given cell type for a particular disease or conditions within the skill of the art.

Accordingly, in specific embodiments, the disclosure relates to a depleting agent (e.g. a CAR cell or an antibody) for use in preventing or reducing the risk of severe side effects in a patient having received a cell expressing a first isoform of CD33 as described above, wherein said patient have native cells expressing a second isoform of CD33, and wherein said depleting agent comprising at least a second antigen-binding region which binds specifically to said first isoform of CD33 and does not bind or binds substantially weaker to said second isoform of CD33.

In another aspect, the present disclosure relates to a kit for expressing a first isoform CD33 as describe above into a cell, said kit comprising a gene editing enzyme, such as guide RNA in combination with a Cas protein, base editor or prime editor, nucleic acid construct, expression vector as described above or isolated cell according to the present disclosure.

### EXAMPLES

### Example 1: Generation of Anti-CD33 Fabs and MAbs

Eight different anti-CD33 antibodies were generated in Fab and MAb format based on publicly available sequence information or sources.

Variable chains and CDRs (Kabat) of the antibodies (Refmab #1-#6) are shown in Table 2. Refmab #7 (WM53) is a mouse monoclonal antibody available from BioLegend (#303402). Refmab #8 (2337A) is a rabbit monoclonal antibody available from R&D Systems (#MAB11373-100).

TABLE 2

| | | |
|---|---|---|
| SEQ ID No. | Comment | Sequence |

Refmab #1

| | | |
|---|---|---|
| 2 | VH | EVQLVQSGAEVKKPGSSVKVSCKASGYTITDSNIHWVRQAPGQS |
| | | LEWIGYIYPYNGGTDYNQKFKNRATLTVDNPTNTAYMELSSLRS |
| | | EDTAFYYCVNGNPWLAYWGQGTLVTVSS |
| 3 | VL | DIQLTQSPSTLSASVGDRVTITCRASESLDNYGIRFLTWFQQKPG |
| | | KAPKLLMYAASNQGSGVPSRFSGSGSGTEFTLTISSLQPDDFATY |
| | | YCQQTKEVPWSFGQGTKVEVK |
| 4 | HCDR1 | DSNIH |
| 5 | HCDR2 | YIYPYNGGTDYNQKFKN |
| 6 | HCDR3 | GNPWLAY |
| 7 | LCDR1 | RASESLDNYGIRFLT |
| 8 | LCDR2 | AASNQGS |
| 9 | LCDR3 | QQTKEVPWS |

Refmab #2

| | | |
|---|---|---|
| 10 | VH | QVQLVQSGAEVKKPGSSVKVSCKASGGTFSDYAISWVRQAPGQ |
| | | GLEWMGRIIPILGVADYAQKFQGRVTITADKSTRTAYMELSSLRS |
| | | EDTAVYYCARNWADAFDIWGQGTMVTVSS |
| 11 | VL | DIQLTQSPSSLSASVGDRVTITCRASQGISSVLAWYQQKPGKAPK |
| | | LLIYDASSLESGVPSRFSGSGSGTDFTLTISSLOPEDFATYYCQQFD |
| | | SSITFGQGTKLEIK |
| 12 | HCDR1 | DYAIS |
| 13 | HCDR2 | RIIPILGVADYAQKFQG |
| 14 | HCDR3 | NWADAFDI |
| 15 | LCDR1 | RASQGISSVLA |
| 16 | LCDR2 | DASSLES |
| 17 | LCDR3 | QQFDSSIT |

Refmab #3

| | | |
|---|---|---|
| 18 | VH | QVQLVQSGAEVKKPGSSVKVSCKASGYTFTDYNMHWVRQAPG |
| | | QGLEWIGYIYPYNGGTGYNQKFKSKATITADESTNTAYMELSSLR |
| | | SEDTAVYYCARGRPAMDYWGQGTLVTVSS |
| 19 | VL | DIQMTQSPSSLSASVGDRVTITCRASESVDNYGISFMNWFQQK |
| | | PGKAPKLLIYAASNQGSGVPSRFSGSGSGTDFTLTISSLQPDDFAT |
| | | YYCQQSKEVPWTFGQGTKVEIK |
| 20 | HCDR1 | DYNMH |
| 21 | HCDR2 | YIYPYNGGTGYNQKFKS |
| 22 | HCDR3 | GRPAMDY |
| 23 | LCDR1 | RASESVDNYGISFMN |
| 8 | LCDR2 | AASNQGS |
| 24 | LCDR3 | QQSKEVPWT |

Refmab #4

| | | |
|---|---|---|
| 25 | VH | QVQLVQSGAEVKKPGESVKVSCKASGYTFTNYGMNWVKQAPG |
| | | QGLEWMGWINTYTGEPTYADKFQGRVTMTTDTSTSTAYMEIR |
| | | NLGGDDTAVYYCARWSWSDGYYVYFDYWGQGTSVTVSS |
| 26 | VL | DIVMTQSPDSLTVSLGERTTINCKSSQSVLDSSTNKNSLAWYQQ |
| | | KPGQPPKLLLSWASTRESGIPDRFSGSGSGTDFTLTIDSPQPEDS |
| | | ATYYCQQSAHFPITFGQGTRLEIK |
| 27 | HCDR1 | NYGMN |
| 28 | HCDR2 | WINTYTGEPTYADKFQG |
| 29 | HCDR3 | WSWSDGYYVYFDY |
| 30 | LCDR1 | KSSQSVLDSSTNKNSLA |
| 31 | LCDR2 | WASTRES |
| 32 | LCDR3 | QQSAHFPIT |

Refmab #5

| | | |
|---|---|---|
| 33 | VH | QVQLVQSGAEVKKPGASVKVSCKASGYTFTNYDINWVRQAPG |
| | | QGLEWIGWIYPGDGSTKYNEKFKAKATLTADTSTSTAYMELRSL |
| | | RSDDTAVYYCASGYEDAMDYWGQGTTVTVSS |
| 34 | VL | DIQMTQSPSSLSASVGDRVTINCKASQDINSYLSWFQQKPGKAP |
| | | KTLIYRANRLVDGVPSRFSGSGSGQDYTLTISSLOPEDFATYYCLQ |
| | | YDEFPLTFGGGTKVEIK |
| 35 | HCDR1 | NYDIN |
| 36 | HCDR2 | WIYPGDGSTKYNEKFKA |
| 37 | HCDR3 | GYEDAMDY |
| 38 | LCDR1 | KASQDINSYLS |
| 39 | LCDR2 | RANRLVD |
| 40 | LCDR3 | LQYDEFPLT |

Refmab #6

| | | |
|---|---|---|
| 41 | VH | QVQLQQPGAEVVKPGASVKMSCKASGYTFTSYYIHWIKQTPGQ |
| | | GLEWVGVIYPGNDDISYNQKFQGKATLTADKSSTTAYMQLSSLT |
| | | SEDSAVYYCAREVRLRYFDVWGQGTTVTVSS |
| 42 | VL | EIVLTQSPGSLAVSPGERVTMSCKSSQSVFFSSSQKNYLAWYQQI |
| | | PGQSPRLLIYWASTRESGVPDRFTGSGSGTDFTLTISSVQPEDLAI |
| | | YYCHQYLSSRTFGQGTKLEIK |
| 43 | HCDR1 | SYYIH |
| 44 | HCDR2 | VIYPGNDDISYNQKFQG |
| 45 | HCDR3 | EVRLRYFDV |
| 46 | LCDR1 | KSSQSVFFSSSQKNYLA |
| 31 | LCDR2 | WASTRES |
| 47 | LCDR3 | HQYLSSRT |

Additional features of the antibodies used, as well as the formats and isotypes of the full length antibodies are shown in Table 3.

TABLE 3

| Antibody | Format/isotype | Source |
|---|---|---|
| Refmab #1 | human IgG1 | Absolute Antibody (hP67.6; Cat. |
| | | No. Ab00283-10.0) |
| Refmab #2 | human IgG1 Fc silent | Sequence taken from U.S. Pat. |
| | (PG-LALA) | No. 9,079,958 |
| Refmab #3 | human IgG1 Fc silent | Sequence taken from U.S. Pat. |
| | (PG-LALA) | No. 5,585,089 |
| Refmab #4 | human IgG1 Fc silent | Sequence taken from U.S. Pat. |
| | (PG-LALA) | No. 10,933,132 |
| Refmab #5 | human IgG1 Fc silent | Sequence taken from U.S. Pat. |
| | (PG-LALA) | No. 9,453,046 |
| Refmab #6 | human IgG1 Fc silent | Sequence taken from U.S. Pat. |
| | (PG-LALA) | No. 7,342,110 |
| Refmab #7 | mouse IgG1 | BioLegend (WM53; Cat. No. |
| | | 303402) |
| Refmab #8 | rabbit IgG | R&D Systems (2337A; Cat. No. |
| | | MAB11373-100) |

### Example 2: Binding of MAbs to CD33 and Optimization of Assay Conditions

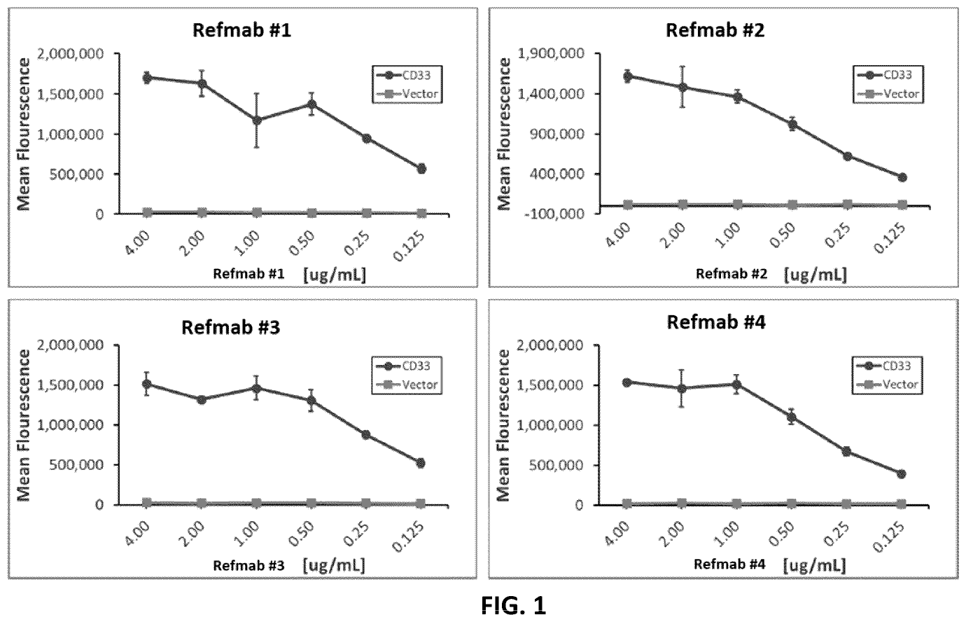

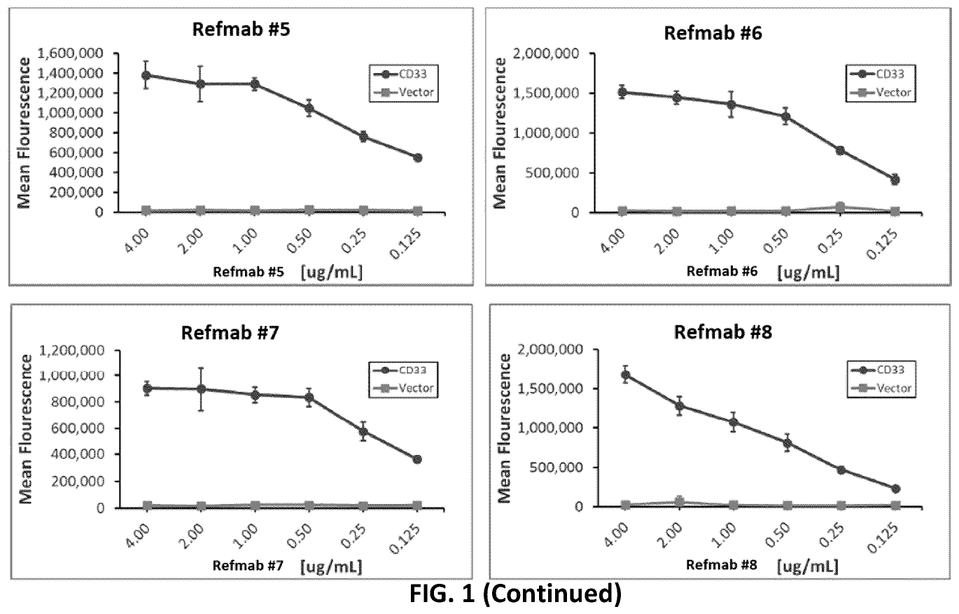

HEK-293T cells were transfected with a construct containing wild-type CD33 (SEQ ID No. 1) or with an empty vector. Binding of the antibodies to the transfected cells and the optimal assay conditions were evaluated in 384-well format. Detection of cellular expression was measured via high-throughput flow cytometry. Serial dilutions of each antibody were tested for immunoreactivity against cells expressing CD33 or vector alone. The optimal screening concentration for each antibody was determined based on the raw signal values and signal-to-background calculations. Results are shown in FIG. 1. Each point represents the mean of four replicates.

All antibodies in Mab format did bind to human CD33 in a concentration dependent manner. Cell transfected with the empty vector did not show significant binding to anti-human CD33 antibodies.

Optimized assay conditions for flow cytometry are shown in Table 4 for Refmab's #1-#4 and in Table 5 for Refmab's #5-#8.

TABLE 4

| Parameter | Refmab #1 | Refmab #2 | Refmab #3 | Refmab #4 |
|---|---|---|---|---|
| Cell Type | HEK293-T | HEK293-T | HEK293-T | HEK293-T |
| Fixative | None | None | None | None |
| Blocking Buffer | 10% goat serum | 10% goat serum | 10% goat serum | 10% goat serum |

Primary antibody

| Name | Refmab #1 | | Refmab#2 | | Refmab#3 | | Refmab#4 | |
|---|---|---|---|---|---|---|---|---|
| Target | CD33 | | CD33 | | CD33 | | CD33 | |
| Optimal conc. | 0.25 | μg/ml | 0.50 | μg/ml | 0.25 | μg/ml | 0.25 | μg/ml |
| Incubation (25° C.) | 60 | min | 60 | min | 60 | min | 60 | min |

Secondary antibody

| Target | Human IgG | | Human IgG | | Human IgG | | Human IgG | |
|---|---|---|---|---|---|---|---|---|
| Optimal conc. | 1:400 (3.75 | | 1:400 (3.75 | | 1:400 (3.75 | | 1:400 (3.75 | |
| | μg/ml) | | μg/ml) | | μg/ml) | | μg/ml) | |
| Incubation (25° C.) | 30 | min | 30 | min | 30 | min | 30 | min |

| | | | | |
|---|---|---|---|---|
| Manufacturer | Jackson | Jackson | Jackson | Jackson |
| | ImmunoResearch | ImmunoResearch | ImmunoResearch | ImmunoResearch |
| Catalogue # | 109-545-003 | 109-545-003 | 109-545-003 | 109-545-003 |
| Antibody ID | AlexaFluor ® 488 | AlexaFluor ® 488 | AlexaFluor ® 488 | AlexaFluor ® 488 |
| | AffiniPure Goat | AffiniPure Goat | AffiniPure Goat | AffiniPure Goat |
| | anti-Human IgG | anti-Human IgG | anti-Human IgG | anti-Human IgG |
| | (H + L) | (H + L) | (H + L) | (H + L) |
| Wash buffer | PBS (Ca2+, Mg2+ | PBS (Ca2+, Mg2+ | PBS (Ca2+, Mg2+ | PBS (Ca2+, Mg2+ |
| | free) | free) | free) | free) |
| Signal:Background | 44:1 | 59:1 | 35:1 | 38:1 |

TABLE 5

| Parameter | Refmab #5 | Refmab #6 | Refmab #7 | Refmab #8 |
|---|---|---|---|---|
| Cell Type | HEK293-T | HEK293-T | HEK293-T | HEK293-T |
| Fixative | None | None | None | None |
| Blocking Buffer | 10% goat serum | 10% goat serum | 10% goat serum | 10% goat serum |

Primary antibody

| Name | Refmab #5 | | Refmab#6 | | Refmab#7 | | Refmab#8 | |
|---|---|---|---|---|---|---|---|---|
| Target | CD33 | | CD33 | | CD33 | | CD33 | |
| Optimal conc. | 0.25 | μg/ml | 0.50 | μg/ml | 0.25 | μg/ml | 0.25 | μg/ml |
| Incubation (25° C.) | 60 | min | 60 | min | 60 | min | 60 | min |

Secondary antibody

| Target | Human IgG | | Human IgG | | Mouse IgG | | Rabbit IgG | |
|---|---|---|---|---|---|---|---|---|
| Optimal conc. | 1:400 (3.75 | | 1:400 (3.75 | | 1:400 (3.75 | | 1:400 (3.75 | |
| | μg/ml) | | μg/ml) | | μg/ml) | | μg/ml) | |
| Incubation (25° C.) | 30 | min | 30 | min | 30 | min | 30 | min |

| | | | | |
|---|---|---|---|---|
| Manufacturer | Jackson | Jackson | Jackson | Jackson |
| | ImmunoResearch | ImmunoResearch | ImmunoResearch | ImmunoResearch |
| Catalogue # | 109-545-003 | 109-545-003 | 115-545-003 | 111-545-003 |
| Antibody ID | AlexaFluor ® 488 | AlexaFluor ® 488 | AlexaFluor ® 488 | AlexaFluor ® 488 |
| | AffiniPure Goat | AffiniPure Goat | AffiniPure Goat | AffiniPure Goat |
| | anti-Human IgG | anti-Human IgG | anti-Mouse IgG | anti-Rabbit IgG |
| | (H + L) | (H + L) | (H + L) | (H + L) |
| Wash buffer | PBS (Ca2+, | PBS (Ca2+, | PBS (Ca2+, | PBS (Ca2+, |
| | Mg2+ free) | Mg2+ free) | Mg2+ free) | Mg2+ free) |
| Signal:Background | 37:1 | 57:1 | 32:1 | 30:1 |

### Example 3: Binding of Fabs to CD33 and Optimization of Assay Conditions

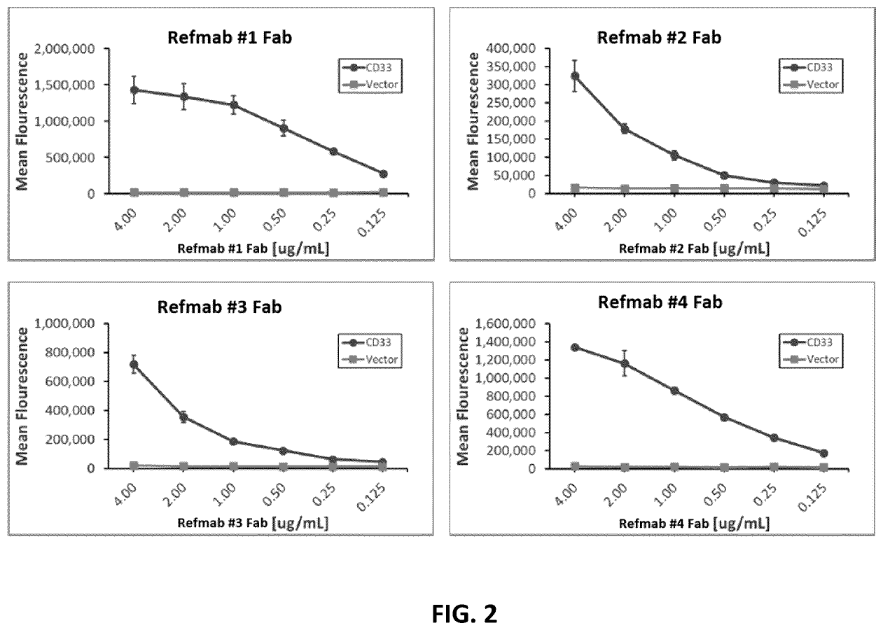

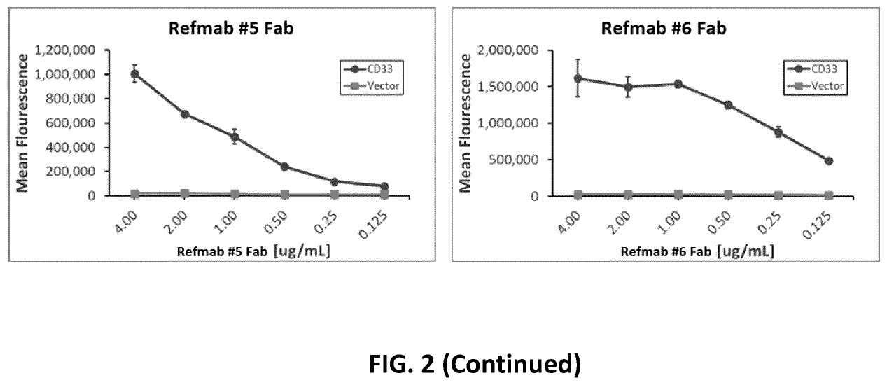

Experiments were performed similar as described in Example 2, except that Fab fragments were tested instead of full-length antibodies. Serial dilutions of each Fab were tested for immunoreactivity against cells expressing wild-type CD33 or vector alone. The optimal screening concentration for the Fabs was determined based on the raw signal values and signal-to-background calculations. Results are shown in FIG. 2. Each point represents the mean of four replicates.

All tested antibodies in Fab format did bind to human CD33 in a concentration dependent manner. Cell transfected with the empty vector did not show any binding to anti-human CD33 antibodies.

Optimized assay conditions for high throughput flow cytometry are shown in Table 6 for Refmab's #1-#3 and in Table 7 for Refmab's #4-#6.

TABLE 6

| Parameter | Refmab #1 | Refmab #2 | Refmab #3 |
|---|---|---|---|
| Cell Type | HEK293-T | HEK293-T | HEK293-T |
| Fixative | None | None | None |
| Blocking Buffer | 10% goat serum | 10% goat serum | 10% goat serum |

Primary antibody

| Name | Refmab #1 | | Refmab#2 | | Refmab#3 | |
|---|---|---|---|---|---|---|
| Target | CD33 | | CD33 | | CD33 | |
| Optimal conc. | 0.25 | μg/ml | 1.00 | μg/ml | 1.00 | μg/ml |
| Incubation (25° C.) | 60 | min | 60 | min | 60 | min |

Secondary antibody

| Target | Human F(ab′)2 | | Human F(ab′)2 | | Human F(ab′)2 | |
|---|---|---|---|---|---|---|
| Optimal conc. | 1:200 (7.50 | | 1:200 (7.50 | | 1:200 (7.50 | |
| | μg/ml) | | μg/ml) | | μg/ml) | |
| Incubation (25° C.) | 30 | min | 30 | min | 30 | min |

| | | | |
|---|---|---|---|
| Manufacturer | Jackson | Jackson | Jackson |
| | ImmunoResearch | ImmunoResearch | ImmunoResearch |
| Catalogue # | 109-546-006 | 109-546-006 | 109-546-006 |
| Antibody ID | AlexaFluor ® 488 | AlexaFluor ® 488 | AlexaFluor ® 488 |
| | AffiniPure Goat | AffiniPure Goat | AffiniPure Goat anti- |
| | anti-Human IgG | anti-Human IgG | Human IgG F(ab′)2 |
| | F(ab′)2 fragment | F(ab′)2 fragment | fragment + L) |
| Wash buffer | PBS (Ca2+, Mg2+ | PBS (Ca2+, Mg2+ | PBS (Ca2+, Mg2+ |
| | free) | free) | free) |
| Signal:Background | 33:1 | 7:1 | 12:1 |

TABLE 7

| Parameter | Refmab #4 | Refmab #5 | Refmab #6 |
|---|---|---|---|
| Cell Type | HEK293-T | HEK293-T | HEK293-T |
| Fixative | None | None | None |
| Blocking Buffer | 10% goat serum | 10% goat serum | 10% goat serum |

Primary antibody

| Name | Refmab #4 | | Refmab#5 | | Refmab#6 | |
|---|---|---|---|---|---|---|
| Target | CD33 | | CD33 | | CD33 | |
| Optimal conc. | 0.50 | μg/ml | 0.50 | μg/ml | 0.25 | μg/ml |
| Incubation (25° C.) | 60 | min | 60 | min | 60 | min |

Secondary antibody

| Target | Human F(ab′)2 | | Human F(ab′)2 | | Human F(ab′)2 | |
|---|---|---|---|---|---|---|
| Optimal conc. | 1:200 (7.50 | | 1:200 (7.50 | | 1:200 (7.50 | |
| | μg/ml) | | μg/ml) | | μg/ml) | |
| Incubation (25° C.) | 30 | min | 30 | min | 30 | min |

| | | | |
|---|---|---|---|
| Manufacturer | Jackson | Jackson | Jackson |
| | ImmunoResearch | ImmunoResearch | ImmunoResearch |
| Catalogue # | 109-546-006 | 109-546-006 | 109-546-006 |
| Antibody ID | AlexaFluor ® 488 | AlexaFluor ® 488 | AlexaFluor ® 488 |
| | AffiniPure Goat | AffiniPure Goat | AffiniPure Goat anti- |
| | anti-Human IgG | anti-Human IgG | Human IgG F(ab′)2 |
| | F(ab′)2 fragment | F(ab′)2 fragment | fragment + L) |
| Wash buffer | PBS (Ca2+, Mg2+ | PBS (Ca2+, Mg2+ | PBS (Ca2+, Mg2+ |
| | free) | free) | free) |
| Signal:Background | 33:1 | 17:1 | 51:1 |

### Example 4: Alanine Scanning

An alanine scan on human CD33 was performed to the determine the residues on CD33 that are involved in binding to the antibodies investigated. The alanine scan was performed via shotgun mutagenesis epitope mapping (Integral Molecular, Philadelphia/PA, USA) as described in Immunology (2014) 143, 13-20. Briefly, a mutation library of CD33 was created by high-throughput, site-directed mutagenesis. Each residue was individually mutated to alanine, with alanine codons mutated to serine. The mutant library was arrayed in 384-well microplates and transiently transfected into HEK293-T. Following transfection, cells were incubated with the indicated antibodies (IgG or Fab) at concentrations pre-determined using an independent immunofluorescence titration curve on wild type CD33. Antibodies were detected using an Alexa Fluor 488-conjugated secondary antibody and mean cellular fluorescence was determined using Intellicyt iQue flow cytometry platform (Intellicyt/Sartorius). Mutated residues were identified as being critical to the antibody epitope if they did not support the reactivity of the test antibody but did support the reactivity of the control antibody, which was in each case another anti-CD33 RefMab, e.g., for RefMab's #1, RefMab #8 was used as control antibody, for RefMab's #2, #3 and #6, RefMab #7 was used as control antibody, and for RefMab's #4 and #5, Refmab #1 was used as control antibody. This counterscreen strategy facilitates the exclusion of mutants that are locally misfolded or that have an expression defect. Binding of each antibody to each mutant clone was determined in duplicates. For each point, background fluorescence was subtracted from the raw data, which were then normalized to antibody reactivity with wild type CD33.

Since library screens of very high-affinity antibodies sometimes fail to yield critical residues for antibody binding, high-affinity antibodies were converted into Fab format to weaken binding sufficiently to allow identification of critical residues for binding. For cases where Fab screens under standard conditions are still insufficient to identify critical residues for binding, high stringency conditions were implemented. These conditions include combinations of increased pH, increased salinity, increased temperature, and/or increased wash time. Antibodies that required high stringency conditions are denoted “HS”.

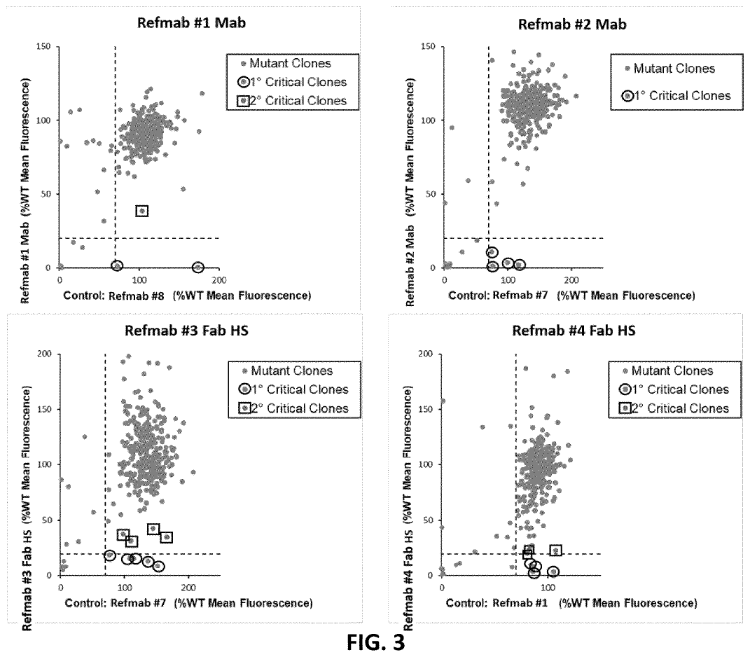

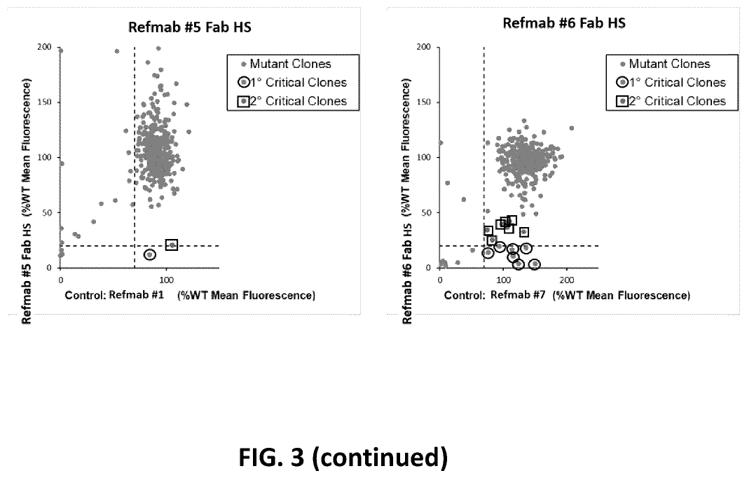

For each mutant clone, the mean binding value was plotted as a function of expression (represented by control reactivity). See FIG. 3. To identify preliminary primary critical clones (circled), a threshold (dashed lines) of >70% wild-type binding to control antibody and <20% wild-type binding to test antibody was applied. Secondary clones (squared) are highlighted for clones that did not meet the set thresholds but whose decreased binding activity and proximity to critical residues suggested that the mutated residue may be part of the antibody epitope.

The result of the alanine scan is shown in Table 8. Mean binding reactivities (and ranges) are listed for all identified critical residues. Critical residues for antibody binding (outlined in dark grey) were residues whose mutations led to a binding of the test Abs of less than 20%. Additional secondary residues (outlined in light grey) were identified that led to a binding of the test Abs of between 20 and 40% as compared to the wild type CD33.

TABLE 8

| | Refmab #1 | | Refmab #2 | | Refmab #3 | | Refmab #4 | |
|---|---|---|---|---|---|---|---|---|
| | Mean % | Range % | Mean % | Range % | Mean % | Range % | Mean % | Range % |
| | WT | WT | WT | WT | WT | WT | WT | WT |
| Mutation | Binding | Binding | Binding | Binding | Binding | Binding | Binding | Binding |
| D18A | 78.6 | 8.2 | 119.1 | 7.6 | 37.8 | 32.9 | 109.7 | 36.0 |
| P19A | 80.8 | 14.6 | 111.2 | 4.3 | 75.2 | 3.9 | 20.4 | 6.8 |
| N20A | 1.2 | 0.1 | 120.6 | 19.4 | 34.9 | 43.5 | 157.9 | 16.7 |
| F21A | 0.4 | 0.5 | 120.5 | 17.5 | 13.6 | 9.1 | 43.6 | 9.6 |
| W22A | 79.4 | 4.0 | 109.9 | 29.9 | 58.6 | 82.3 | 186.9 | 14.0 |
| L23A | 82.7 | 17.4 | 83.4 | 1.0 | 91.5 | 20.2 | 23.7 | 8.3 |
| Q24A | 74.1 | 1.6 | 102.8 | 15.8 | 101.0 | 171.1 | 68.3 | 5.1 |
| V25A | 90.7 | 7.7 | 87.1 | 8.8 | 108.9 | 22.4 | 49.1 | 6.9 |
| Q26A | 81.4 | 7.3 | 110.9 | 1.4 | 123.5 | 18.8 | 63.1 | 22.7 |
| E27A | 82.5 | 4.4 | 111.2 | 20.5 | 152.8 | 54.1 | 42.9 | 42.1 |
| S28A | 93.9 | 2.0 | 117.2 | 0.9 | 193.1 | 225.6 | 79.9 | 21.9 |
| V29A | 101.8 | 11.4 | 100.1 | 0.8 | 115.4 | 48.2 | 84.4 | 33.3 |
| T30A | 89.3 | 13.3 | 92.1 | 1.8 | 104.2 | 31.8 | 103.8 | 8.6 |
| V31A | 87.0 | 14.3 | 106.1 | 16.0 | 127.4 | 5.8 | 97.9 | 16.3 |
| Q32A | 87.0 | 10.9 | 108.2 | 26.8 | 132.8 | 17.3 | 83.0 | 2.7 |
| E33A | 86.4 | 14.4 | 119.9 | 14.0 | 94.9 | 19.5 | 87.1 | 6.8 |
| G34A | 99.5 | 21.0 | 105.0 | 1.0 | 99.4 | 35.0 | 93.4 | 12.5 |
| L35A | 89.1 | 6.0 | 118.6 | 7.6 | 101.5 | 27.5 | 94.5 | 13.7 |
| C36A | 72.6 | 1.1 | 116.7 | 2.3 | 102.4 | 10.6 | 108.2 | 37.1 |
| V37A | 78.7 | 15.7 | 118.4 | 12.6 | 149.9 | 46.0 | 78.7 | 28.8 |
| L38A | 94.8 | 20.0 | 111.7 | 4.8 | 192.1 | 12.8 | 96.8 | 22.2 |
| V39A | 80.0 | 5.8 | 110.9 | 21.1 | 122.5 | 64.9 | 58.4 | 61.5 |
| P40A | 74.5 | 5.9 | 101.0 | 5.7 | 122.0 | 36.2 | 43.0 | 14.5 |
| C41A | 14.0 | 2.2 | 10.6 | 2.5 | 30.7 | 2.4 | 9.8 | 0.3 |
| T42A | 105.1 | 9.8 | 111.8 | 16.2 | 197.8 | 45.5 | 67.4 | 18.3 |
| F43A | 66.3 | 10.4 | 10.6 | 1.5 | 49.3 | 4.3 | 7.8 | 6.6 |
| F44A | 85.1 | 3.7 | 1.2 | 1.6 | 18.9 | 11.8 | 151.6 | 23.8 |
| H45A | 38.7 | 2.5 | 73.6 | 2.8 | 55.1 | 71.7 | 134.5 | 57.5 |
| P46A | 64.8 | 20.2 | 103.9 | 16.8 | 8.8 | 0.7 | 135.4 | 2.4 |
| I47A | 84.2 | 20.7 | 2.1 | 0.4 | 165.3 | 4.1 | 144.3 | 24.4 |
| P48A | 86.2 | 0.6 | 109.1 | 8.3 | 167.1 | 33.1 | 8.4 | 3.0 |
| Y49A | 105.6 | 11.3 | 44.2 | 0.6 | 86.6 | 28.3 | 3.1 | 0.7 |
| Y50A | 86.0 | 6.9 | 140.7 | 0.4 | 109.4 | 13.8 | 3.5 | 0.7 |
| D51A | 82.4 | 7.0 | 59.0 | 0.8 | 125.7 | 24.8 | 36.0 | 10.9 |
| K52A | 84.2 | 6.7 | 94.9 | 2.5 | 80.3 | 8.2 | 9.8 | 5.3 |
| N53A | 94.7 | 2.1 | 96.0 | 2.3 | 116.0 | 35.7 | 69.0 | 13.1 |
| S54A | 87.6 | 3.9 | 111.6 | 0.4 | 106.5 | 3.9 | 99.9 | 20.2 |
| P55A | 98.8 | 23.9 | 120.4 | 5.9 | 142.5 | 52.2 | 95.4 | 13.4 |
| V56A | 94.3 | 4.1 | 80.7 | 10.6 | 42.6 | 0.7 | 56.8 | 52.9 |
| H57A | 78.2 | 15.4 | 112.2 | 16.8 | 182.1 | 1.8 | 67.3 | 46.4 |
| G58A | 77.4 | 8.0 | 118.2 | 1.3 | 87.4 | 19.9 | 62.2 | 9.3 |
| Y59A | 93.6 | 7.1 | 95.7 | 5.0 | 144.9 | 63.4 | 97.6 | 32.6 |
| W60A | 0.7 | 0.1 | 2.8 | 1.4 | 28.3 | 38.7 | 1.0 | 1.1 |
| F61A | 95.0 | 9.1 | 134.6 | 26.3 | 304.4 | 82.4 | 131.4 | 31.5 |
| R62A | 121.2 | 17.3 | 124.1 | 1.6 | 91.8 | 42.2 | 104.1 | 13.5 |
| E63A | 107.0 | 27.0 | 107.3 | 0.4 | 124.5 | 35.9 | 84.1 | 22.1 |
| G64A | 90.6 | 33.9 | 113.9 | 3.0 | 96.6 | 23.4 | 93.9 | 17.1 |
| A65S | 88.5 | 12.8 | 111.6 | 21.2 | 143.3 | 47.9 | 84.0 | 35.3 |
| I66A | 96.6 | 14.7 | 117.7 | 11.8 | 126.5 | 11.8 | 108.4 | 13.7 |
| I67A | 98.1 | 22.9 | 122.1 | 10.2 | 141.8 | 55.8 | 67.2 | 22.8 |
| S68A | 91.3 | 5.3 | 111.6 | 2.2 | 115.1 | 31.7 | 90.4 | 25.3 |
| R69A | 94.2 | 6.6 | 137.9 | 7.0 | 188.2 | 15.2 | 118.2 | 78.3 |
| D70A | 81.3 | 9.5 | 109.5 | 2.1 | 131.1 | 37.3 | 82.7 | 31.6 |
| S71A | 107.5 | 12.2 | 111.8 | 5.0 | 117.8 | 4.0 | 99.4 | 22.2 |
| P72A | 99.9 | 2.4 | 113.7 | 11.3 | 107.9 | 39.8 | 94.6 | 19.2 |
| V73A | 73.6 | 24.4 | 99.1 | 2.4 | 148.8 | 63.9 | 67.2 | 12.2 |
| A74S | 83.7 | 8.6 | 91.7 | 32.4 | 114.8 | 8.7 | 72.5 | 14.6 |
| T75A | 100.8 | 0.4 | 114.3 | 0.0 | 118.0 | 10.4 | 93.8 | 13.1 |
| N76A | 61.8 | 14.4 | 106.0 | 2.7 | 147.8 | 135.7 | 34.7 | 19.6 |
| K77A | 95.6 | 2.1 | 123.3 | 17.9 | 191.7 | 81.9 | 122.6 | 27.9 |
| L78A | 87.4 | 5.8 | 108.4 | 8.9 | 113.3 | 4.0 | 110.9 | 6.4 |
| D79A | 88.1 | 3.1 | 101.8 | 1.7 | 104.7 | 10.8 | 74.5 | 26.7 |
| Q80A | 91.6 | 7.0 | 112.1 | 20.2 | 123.1 | 13.6 | 124.2 | 21.0 |
| E81A | 83.0 | 15.7 | 106.7 | 4.9 | 114.0 | 20.3 | 90.6 | 12.9 |
| V82A | 99.6 | 3.9 | 115.5 | 1.6 | 141.0 | 55.6 | 144.5 | 22.9 |
| Q83A | 87.1 | 2.9 | 106.8 | 16.8 | 103.2 | 36.2 | 103.9 | 21.9 |
| E84A | 92.2 | 18.7 | 114.5 | 16.4 | 137.2 | 44.4 | 120.0 | 13.4 |
| E85A | 88.9 | 17.7 | 113.9 | 12.5 | 112.5 | 7.3 | 110.2 | 10.2 |
| T86A | 79.1 | 1.1 | 113.1 | 11.8 | 111.8 | 24.0 | 102.7 | 22.8 |
| Q87A | 91.0 | 4.0 | 117.6 | 6.5 | 161.1 | 39.2 | 94.2 | 10.7 |
| G88A | 96.1 | 21.8 | 109.9 | 9.9 | 88.3 | 27.3 | 97.7 | 12.7 |
| R89A | 77.6 | 7.7 | 98.7 | 3.9 | 125.9 | 20.9 | 71.6 | 63.4 |
| F90A | 73.9 | 4.0 | 103.6 | 2.3 | 137.7 | 14.4 | 82.3 | 30.7 |
| R91A | 82.0 | 1.3 | 119.1 | 4.8 | 149.7 | 22.5 | 100.1 | 26.1 |
| L92A | 75.2 | 34.2 | 106.4 | 9.7 | 128.8 | 42.2 | 85.2 | 22.7 |
| L93A | 88.6 | 7.9 | 103.0 | 3.4 | 119.9 | 17.2 | 63.9 | 27.1 |
| G94A | 88.3 | 3.8 | 107.8 | 2.2 | 114.6 | 55.1 | 59.0 | 6.6 |
| D95A | 80.5 | 13.7 | 110.2 | 13.7 | 102.7 | 25.0 | 90.9 | 19.5 |
| P96A | 84.9 | 1.9 | 102.3 | 2.4 | 139.7 | 93.6 | 27.4 | 2.4 |
| S97A | 92.5 | 10.0 | 131.5 | 16.6 | 90.4 | 34.6 | 120.7 | 20.1 |
| R98A | 99.1 | 6.9 | 122.2 | 6.6 | 101.6 | 15.6 | 127.6 | 16.6 |
| N99A | 80.2 | 1.4 | 56.8 | 9.3 | 98.6 | 28.3 | 43.5 | 5.6 |
| N100A | 94.8 | 0.5 | 97.0 | 4.4 | 107.3 | 45.4 | 123.3 | 30.0 |
| C101A | 1.1 | 0.4 | 0.6 | 0.6 | 13.6 | 19.9 | 1.5 | 0.7 |
| S102A | 85.8 | 35.7 | 89.6 | 10.9 | 75.9 | 13.8 | 73.6 | 4.8 |
| L103A | 96.3 | 12.1 | 110.3 | 0.3 | 112.9 | 31.3 | 85.9 | 5.1 |
| S104A | 89.0 | 16.0 | 108.9 | 6.3 | 101.6 | 31.4 | 86.4 | 34.0 |
| I105A | 31.5 | 3.0 | 43.7 | 2.9 | 64.7 | 29.5 | 21.6 | 0.4 |
| V106A | 88.5 | 10.6 | 112.2 | 1.1 | 85.7 | 4.4 | 110.1 | 35.9 |
| D107A | 87.7 | 22.5 | 108.7 | 18.4 | 92.5 | 34.2 | 93.0 | 30.2 |
| A108S | 85.3 | 25.2 | 104.5 | 7.2 | 98.0 | 7.9 | 86.5 | 6.5 |
| R109A | 100.8 | 0.5 | 104.0 | 5.1 | 112.2 | 20.0 | 99.5 | 11.6 |
| R110A | 94.9 | 8.9 | 109.3 | 5.8 | 99.1 | 20.8 | 88.4 | 13.0 |
| R111A | 100.5 | 17.3 | 107.5 | 8.1 | 93.4 | 23.1 | 94.3 | 0.5 |
| D112A | 51.8 | 3.4 | 58.7 | 1.5 | 77.5 | 12.2 | 36.0 | 9.8 |
| N113A | 96.0 | 8.9 | 91.0 | 4.6 | 81.4 | 31.2 | 45.1 | 9.0 |
| G114A | 96.9 | 16.7 | 116.4 | 1.2 | 92.6 | 38.5 | 109.7 | 9.6 |
| S115A | 86.9 | 13.5 | 93.2 | 2.0 | 77.2 | 33.4 | 36.4 | 13.8 |
| Y116A | 0.9 | 1.7 | 0.7 | 1.2 | 5.3 | 7.7 | 5.4 | 0.8 |
| F117A | 98.7 | 11.1 | 111.4 | 28.3 | 166.5 | 58.1 | 142.5 | 7.2 |
| F118A | 0.1 | 0.1 | 0.6 | 0.8 | 8.2 | 0.9 | 6.0 | 0.5 |
| R119A | 109.4 | 8.9 | 129.9 | 13.1 | 1021.1 | 175.7 | 138.4 | 43.5 |
| M120A | 79.8 | 2.1 | 70.7 | 3.8 | 15.7 | 12.5 | 96.8 | 11.1 |
| E121A | 87.7 | 1.6 | 108.7 | 10.8 | 132.1 | 8.9 | 77.3 | 0.4 |
| R122A | 107.4 | 4.4 | 3.6 | 0.5 | 177.4 | 88.2 | 23.0 | 7.5 |
| G123A | 107.3 | 41.2 | 82.0 | 0.2 | 292.1 | 171.2 | 97.9 | 11.5 |
| S124A | 87.5 | 5.0 | 110.7 | 1.3 | 273.5 | 174.0 | 58.8 | 13.8 |
| T125A | 92.4 | 15.0 | 106.7 | 7.7 | 932.4 | 317.6 | 218.0 | 31.9 |
| K126A | 100.0 | 24.5 | 120.6 | 4.4 | 566.5 | 457.9 | 211.5 | 63.3 |
| Y127A | 92.6 | 16.3 | 146.5 | 4.3 | 31.9 | 31.5 | 300.5 | 74.6 |
| S128A | 118.3 | 2.8 | 139.5 | 3.0 | 445.5 | 158.6 | 184.2 | 38.7 |
| Y129A | 53.2 | 23.4 | 137.1 | 34.5 | 15.6 | 17.6 | 319.4 | 43.8 |
| K130A | 105.5 | 6.0 | 123.0 | 3.2 | 151.8 | 23.4 | 180.1 | 1.2 |
| S131A | 85.9 | 12.8 | 123.4 | 21.8 | 117.3 | 28.4 | 133.9 | 27.0 |
| P132A | 106.7 | 9.8 | 102.0 | 4.3 | 74.5 | 13.2 | 70.1 | 1.7 |
| Q133A | 95.6 | 11.2 | 130.6 | 19.7 | 101.4 | 17.7 | 96.0 | 22.5 |
| L134A | 95.4 | 4.7 | 99.6 | 3.4 | 81.4 | 15.5 | 82.2 | 10.8 |
| S135A | 98.6 | 6.7 | 119.7 | 12.4 | 107.4 | 29.5 | 109.4 | 20.7 |
| V136A | 64.2 | 0.8 | 99.8 | 5.1 | 106.5 | 45.2 | 47.7 | 9.1 |
| H137A | 83.8 | 8.9 | 120.4 | 16.1 | 100.3 | 7.1 | 107.7 | 26.3 |
| V138A | 72.3 | 2.4 | 98.5 | 3.8 | 129.0 | | 54.1 | 25.8 |
| T139A | 89.9 | 17.7 | 123.1 | 9.3 | 142.8 | 68.9 | 84.3 | 25.6 |
| D140A | 88.7 | 16.4 | 108.6 | 12.4 | 119.4 | 72.0 | 96.1 | 23.5 |
| L141A | 103.4 | 15.2 | 113.0 | 4.8 | 153.2 | 64.4 | 93.7 | 30.8 |
| T142A | 85.1 | 14.8 | 104.0 | 11.4 | 132.7 | 62.2 | 88.8 | 34.8 |
| H143A | 88.8 | 16.9 | 104.3 | 3.8 | 97.9 | 19.9 | 105.6 | 34.0 |
| R144A | 110.5 | 9.2 | 112.1 | 16.8 | 112.1 | 20.7 | 99.7 | 31.8 |
| P145A | 89.3 | 0.3 | 104.6 | 3.2 | 75.2 | 17.5 | 78.8 | 12.2 |
| K146A | 78.5 | 3.9 | 115.2 | 5.2 | 118.0 | 9.1 | 121.5 | 9.8 |
| I147A | 71.6 | 7.6 | 92.2 | 2.4 | 67.1 | 1.1 | 58.7 | 15.0 |
| L148A | 83.1 | 6.9 | 119.2 | 5.9 | 108.5 | 20.7 | 112.6 | 19.2 |
| I149A | 87.5 | 1.7 | 115.7 | 10.5 | 101.3 | 18.0 | 99.9 | 5.0 |
| P150A | 78.4 | 17.3 | 111.7 | 8.2 | 101.7 | 2.0 | 103.1 | 10.8 |
| G151A | 81.1 | 37.0 | 132.5 | 39.5 | 107.6 | 18.7 | 101.0 | 16.2 |
| T152A | 86.3 | 13.6 | 116.8 | 12.0 | 155.1 | 82.7 | 109.5 | 15.7 |
| L153A | 75.7 | 7.5 | 121.3 | 0.4 | 143.6 | 46.9 | 92.5 | 19.2 |
| E154A | 88.0 | 3.2 | 112.2 | 7.8 | 126.3 | 53.0 | 84.4 | 22.6 |
| P155A | 84.4 | 10.9 | 126.1 | 11.2 | 113.8 | 57.1 | 110.7 | 29.6 |
| G156A | 83.4 | 16.6 | 99.4 | 3.1 | 116.1 | 49.9 | 67.2 | 21.1 |
| H157A | 86.6 | 21.4 | 115.4 | 18.8 | 137.7 | 8.4 | 97.4 | 40.2 |
| S158A | 87.1 | 16.2 | 110.7 | 13.6 | 110.8 | 46.8 | 98.0 | 21.2 |
| K159A | 119.3 | 42.1 | 133.9 | 8.2 | 110.4 | 12.5 | 117.7 | 15.8 |
| N160A | 82.3 | 6.5 | 106.6 | 11.2 | 99.3 | 1.9 | 101.5 | 10.9 |
| L161A | 87.0 | 16.9 | 113.7 | 0.5 | 115.5 | 13.8 | 113.6 | 18.4 |
| T162A | 86.9 | 3.4 | 102.4 | 2.8 | 109.2 | 39.4 | 79.9 | 7.9 |
| C163A | 84.6 | 5.9 | 97.7 | 6.3 | 65.7 | 45.4 | 43.3 | 1.6 |
| S164A | 100.9 | 0.9 | 116.9 | 8.0 | 96.3 | 25.6 | 112.2 | 8.9 |
| V165A | 91.6 | 8.2 | 124.1 | 14.7 | 116.0 | 7.1 | 117.0 | 25.0 |
| S166A | 98.3 | 12.9 | 113.7 | 9.4 | 130.8 | 12.1 | 109.5 | 33.6 |
| W167A | 89.4 | 4.7 | 108.8 | 2.5 | 129.1 | 45.4 | 111.8 | 36.4 |
| A168S | 88.4 | 21.2 | 109.9 | 2.1 | 99.2 | 25.1 | 99.9 | 25.6 |
| C169A | 75.8 | 27.4 | 106.0 | 14.0 | 115.4 | 56.6 | 69.3 | 19.0 |
| E170A | 90.9 | 18.9 | 122.6 | 1.9 | 148.2 | 73.8 | 95.5 | 31.5 |
| Q171A | 90.8 | 25.2 | 90.0 | 19.0 | 138.5 | 71.9 | 101.8 | 23.5 |
| G172A | 86.0 | 2.8 | 108.9 | 9.6 | 106.9 | 48.4 | 90.7 | 16.4 |
| T173A | 105.9 | 14.8 | 133.8 | 11.9 | 97.0 | 3.0 | 119.3 | 2.0 |
| P174A | 90.9 | 7.0 | 116.7 | 13.1 | 109.6 | 14.3 | 118.4 | 19.1 |
| P175A | 89.4 | 0.3 | 102.7 | 23.7 | 105.9 | 1.8 | 106.1 | 7.7 |
| I176A | 93.5 | 3.0 | 124.0 | 2.2 | 100.2 | 7.6 | 123.8 | 18.4 |
| F177A | 68.4 | 5.1 | 67.5 | 4.1 | 60.4 | 29.2 | 25.4 | 1.0 |
| S178A | 94.0 | 8.1 | 112.6 | 14.8 | 112.4 | 12.7 | 100.8 | 33.1 |
| W179A | 93.5 | 5.9 | 112.8 | 9.1 | 102.8 | 44.6 | 83.1 | 18.6 |
| L180A | 91.3 | 19.7 | 117.9 | 12.8 | 101.7 | 5.7 | 100.7 | 43.3 |
| S181A | 85.5 | 18.0 | 115.7 | 2.7 | 128.1 | 54.7 | 114.1 | 35.2 |
| A182S | 85.8 | 1.8 | 115.3 | 4.6 | 126.8 | 90.5 | 108.4 | 29.2 |
| A183S | 85.4 | 15.0 | 119.3 | 35.0 | 117.1 | 65.5 | 102.6 | 10.7 |
| P184A | 82.9 | 11.0 | 111.0 | 5.1 | 102.6 | 22.1 | 79.4 | 39.1 |
| T185A | 92.3 | 18.2 | 117.8 | 7.2 | 105.1 | 50.8 | 98.9 | 23.4 |
| S186A | 92.5 | 7.5 | 119.7 | 18.3 | 106.0 | 19.0 | 121.2 | 25.7 |
| L187A | 92.3 | 2.5 | 144.3 | 8.9 | 95.5 | 2.4 | 108.2 | 25.9 |
| G188A | 101.8 | 5.3 | 130.1 | 29.2 | 85.1 | 29.9 | 91.3 | 11.2 |
| P189A | 89.2 | 0.9 | 115.6 | 5.6 | 95.5 | 3.1 | 98.2 | 6.6 |
| R190A | 96.6 | 6.8 | 114.4 | 4.9 | 123.9 | 45.2 | 108.6 | 14.9 |
| T191A | 91.4 | 8.3 | 114.1 | 16.9 | 80.0 | 22.9 | 95.1 | 11.9 |
| T192A | 100.6 | 0.0 | 118.6 | 14.5 | 102.2 | 41.1 | 99.7 | 23.6 |
| H193A | 99.9 | 6.9 | 121.6 | 3.7 | 92.0 | 39.1 | 102.9 | 34.7 |
| S194A | 93.7 | 13.0 | 112.3 | 13.3 | 100.9 | 42.1 | 79.0 | 59.6 |
| S195A | 86.9 | 1.8 | 118.7 | 0.0 | 95.2 | 26.6 | 111.5 | 2.6 |
| V196A | 83.9 | 8.9 | 106.0 | 11.5 | 105.4 | 31.9 | 102.4 | 27.3 |
| L197A | 95.5 | 11.3 | 125.0 | 12.8 | 91.4 | 21.2 | 95.3 | 25.1 |
| —, I198A | 86.7 | 23.7 | 114.2 | 13.4 | 91.2 | 28.1 | 94.4 | 25.5 |
| I199A | 98.5 | 11.7 | 125.2 | 3.2 | 112.0 | 4.3 | 114.2 | 12.9 |
| T200A | 85.2 | 4.1 | 110.8 | 9.8 | 82.4 | 10.6 | 88.8 | 1.7 |
| P201A | 85.6 | 7.1 | 110.5 | 1.0 | 90.9 | 16.7 | 102.8 | 18.7 |
| R202A | 86.7 | 6.6 | 111.8 | 20.7 | 128.1 | 28.2 | 88.7 | 14.2 |
| P203A | 103.3 | 10.7 | 109.3 | 10.7 | 105.3 | 8.2 | 112.5 | 29.3 |
| Q204A | 93.7 | 10.2 | 112.3 | 2.1 | 126.1 | 36.8 | 99.8 | 0.1 |
| D205A | 1.6 | 0.7 | 2.8 | 2.2 | 8.1 | 1.4 | 0.1 | 0.5 |
| H206A | 98.8 | 3.5 | 110.0 | 1.8 | 97.3 | 43.6 | 97.7 | 5.9 |
| G207A | 94.1 | 11.1 | 108.6 | 3.2 | 104.6 | 44.1 | 87.1 | 23.1 |
| T208A | 100.6 | 16.2 | 117.1 | 9.0 | 83.6 | 8.1 | 107.8 | 25.2 |
| N209A | 92.1 | 20.6 | 103.8 | 1.2 | 91.7 | 23.2 | 88.1 | 38.2 |
| L210A | 93.0 | 4.5 | 98.2 | 7.9 | 84.0 | 11.8 | 92.3 | 13.5 |
| T211A | 82.4 | 3.7 | 111.0 | 11.7 | 96.2 | 41.9 | 79.4 | 12.7 |
| C212A | 17.2 | 2.2 | 18.5 | 0.0 | 57.3 | 23.0 | 11.0 | 1.0 |
| Q213A | 95.3 | 0.5 | 115.2 | 5.2 | 135.1 | 44.1 | 102.7 | 15.1 |
| V214A | 80.5 | 3.8 | 118.2 | 12.1 | 101.6 | 37.1 | 101.2 | 12.8 |
| K215A | 84.5 | 10.3 | 98.4 | 5.3 | 79.8 | 21.8 | 96.7 | 17.2 |
| F216A | 87.3 | 3.8 | 100.9 | 0.5 | 151.6 | 5.5 | 71.6 | 16.4 |
| A217S | 80.2 | 9.5 | 109.4 | 16.8 | 148.3 | 69.2 | 61.1 | 44.4 |
| G218A | 79.2 | 5.3 | 123.8 | 0.0 | 143.0 | 4.6 | 102.4 | 42.2 |
| A219S | 104.1 | 18.9 | 117.3 | 2.8 | 113.3 | 44.9 | 111.7 | 28.4 |
| G220A | 104.4 | 32.3 | 131.8 | 10.7 | 113.5 | 30.5 | 115.3 | 26.2 |
| V221A | 91.9 | 5.4 | 92.3 | 9.6 | 89.0 | 8.9 | 103.7 | 10.5 |
| T222A | 88.7 | 9.2 | 121.2 | 17.2 | 117.4 | 9.0 | 108.6 | 28.4 |
| T223A | 103.0 | 2.0 | 125.6 | 11.4 | 93.5 | 6.7 | 105.6 | 24.2 |
| E224A | 95.4 | 14.0 | 102.3 | 14.0 | 83.2 | 5.0 | 107.4 | 25.7 |
| R225A | 97.8 | 2.4 | 122.9 | 7.0 | 98.9 | 0.7 | 108.4 | 16.5 |
| T226A | 92.0 | 7.6 | 112.1 | 7.3 | 100.4 | 14.3 | 91.9 | 28.9 |
| I227A | 86.7 | 4.2 | 110.2 | 4.3 | 127.2 | 52.9 | 78.7 | 19.1 |
| Q228A | 99.4 | 10.8 | 120.5 | 3.2 | 156.7 | 37.9 | 112.4 | 42.4 |
| L229A | 93.1 | 18.1 | 112.9 | 1.4 | 150.3 | 12.1 | 89.1 | 24.5 |
| N230A | 83.6 | 6.6 | 96.4 | 4.6 | 108.9 | 26.8 | 63.5 | 23.5 |
| V231A | 100.2 | 40.1 | 111.7 | 4.8 | 153.2 | | 79.3 | 17.7 |
| T232A | 76.6 | 5.8 | 84.8 | 14.3 | 127.0 | 83.9 | 57.4 | 11.6 |
| Y233A | 115.4 | 1.9 | 107.0 | 16.5 | 146.0 | 55.5 | 98.5 | 36.6 |
| V234A | 85.8 | 10.8 | 116.8 | 4.2 | 88.7 | 0.2 | 107.2 | 7.8 |
| P235A | 81.4 | 19.0 | 108.3 | 0.2 | 120.4 | 6.2 | 100.6 | 5.7 |
| Q236A | 89.5 | 9.6 | 111.0 | 0.5 | 103.5 | 1.6 | 102.9 | 20.2 |
| N237A | 98.7 | 4.7 | 111.8 | 10.1 | 85.6 | 42.3 | 72.9 | 37.7 |
| P238A | 101.2 | 11.1 | 119.0 | 17.2 | 122.5 | 52.4 | 117.1 | 8.5 |
| T239A | 79.7 | 21.0 | 111.1 | 1.4 | 105.4 | 39.6 | 102.5 | 23.3 |
| T240A | 110.6 | 20.0 | 111.2 | 7.4 | 97.9 | 46.5 | 76.8 | 49.4 |
| G241A | 102.1 | 13.2 | 110.8 | 4.1 | 128.3 | 16.1 | 109.0 | 13.0 |
| I242A | 77.9 | 15.6 | 98.6 | 1.3 | 127.8 | 66.1 | 104.4 | 26.7 |
| F243A | 86.5 | 8.8 | 102.5 | 13.1 | 106.8 | 14.9 | 69.9 | 35.6 |
| P244A | 84.3 | 1.4 | 112.4 | 1.5 | 137.8 | 72.3 | 100.2 | 16.8 |
| G245A | 84.3 | 9.4 | 111.8 | 8.6 | 127.0 | 62.3 | 94.9 | 32.8 |
| D246A | 85.2 | 4.8 | 113.7 | 5.5 | 115.1 | 21.4 | 103.9 | 26.3 |
| G247A | 98.5 | 16.0 | 110.2 | 0.1 | 106.0 | 45.1 | 113.1 | 17.4 |
| S248A | 88.4 | 10.0 | 93.6 | 10.1 | 132.7 | 91.4 | 99.7 | 19.5 |
| G249A | 89.2 | 7.8 | 127.1 | 7.3 | 132.0 | 40.1 | 110.9 | 28.0 |
| K250A | 86.7 | 13.7 | 108.1 | 4.3 | 145.8 | 66.6 | 106.3 | 33.2 |
| Q251A | 98.8 | 6.6 | 116.6 | 18.8 | 93.3 | 17.3 | 97.5 | 23.0 |
| E252A | 98.8 | 7.2 | 115.5 | 9.7 | 96.3 | 9.1 | 115.3 | 22.7 |
| T253A | 94.5 | 3.5 | 103.8 | 10.3 | 98.1 | 6.7 | 107.6 | 21.3 |
| R254A | 101.0 | 1.4 | 117.7 | 13.9 | 88.4 | 17.4 | 101.2 | 12.1 |
| A255S | 98.8 | 3.8 | 108.7 | 1.5 | 97.7 | 27.5 | 113.1 | 26.4 |
| G256A | 89.8 | 11.0 | 117.4 | 12.3 | 94.9 | 7.8 | 103.8 | 36.2 |
| V257A | 102.1 | 9.0 | 108.2 | 2.5 | 89.0 | 6.2 | 109.6 | 16.4 |
| V258A | 99.8 | 0.7 | 110.7 | 4.5 | 106.3 | 24.8 | 95.8 | 18.2 |
| H259A | 97.4 | 4.6 | 108.7 | 3.6 | 104.2 | 20.6 | 113.7 | 34.8 |
| G260A | 81.9 | 17.2 | 104.7 | 4.3 | 139.8 | 65.4 | 105.6 | 27.6 |
| A261S | 84.1 | 3.1 | 100.3 | 8.6 | 121.1 | 50.0 | 65.8 | 21.3 |
| I262A | 89.7 | 15.0 | 115.4 | 2.6 | 130.0 | 73.9 | 111.3 | 14.8 |
| G263A | 98.9 | 7.9 | 102.4 | 9.2 | 116.3 | 78.0 | 99.9 | 21.5 |
| G264A | 95.0 | 14.5 | 105.1 | 24.5 | 89.0 | 10.7 | 89.9 | 15.0 |
| A265S | 93.2 | 7.7 | 103.6 | 16.0 | 92.4 | 7.3 | 125.8 | 27.6 |
| G266A | 100.2 | 4.6 | 116.0 | 0.6 | 97.8 | 4.3 | 111.7 | 11.7 |
| V267A | 88.3 | 4.2 | 113.8 | 5.6 | 105.8 | 23.9 | 115.9 | 20.0 |
| T268A | 102.5 | 0.2 | 107.3 | 4.3 | 91.5 | 26.1 | 100.1 | 13.9 |
| A269S | 107.3 | 15.9 | 110.7 | 2.6 | 97.4 | 9.1 | 83.6 | 11.3 |
| L270A | 102.3 | 7.8 | 118.6 | 0.2 | 124.6 | 48.4 | 97.3 | 11.4 |
| L271A | 100.5 | 19.7 | 113.3 | 13.3 | 134.8 | 109.4 | 113.2 | 20.3 |
| A272S | 87.6 | 9.8 | 109.8 | 2.4 | 105.3 | 19.3 | 106.7 | 30.1 |
| L273A | 110.3 | 26.7 | 116.8 | 4.1 | 125.1 | 1.8 | 115.3 | 29.8 |
| C274A | 88.4 | 1.1 | 116.8 | 4.6 | 110.9 | 42.5 | 103.2 | 16.7 |
| L275A | 88.7 | 0.7 | 115.3 | 2.4 | 122.6 | 85.1 | 99.3 | 23.3 |
| C276A | 83.4 | 25.6 | 102.6 | 15.4 | 115.3 | 32.7 | 96.9 | 37.4 |
| L277A | 85.5 | 9.0 | 116.7 | 2.8 | 81.9 | 23.5 | 92.2 | 27.4 |
| I278A | 99.1 | 2.3 | 105.8 | 1.1 | 100.2 | 6.9 | 116.3 | 25.7 |
| F279A | 103.6 | 24.4 | 116.5 | 6.0 | 138.0 | 94.9 | 103.0 | 31.0 |
| F280A | 98.4 | 3.5 | 120.3 | 11.2 | 99.0 | 11.2 | 92.3 | 12.9 |
| I281A | 85.0 | 14.8 | 113.6 | 0.4 | 91.0 | 36.0 | 122.2 | 42.5 |
| V282A | 108.6 | 21.7 | 112.5 | 0.2 | 73.2 | 13.9 | 110.7 | 17.4 |
| K283A | 92.7 | 25.1 | 102.5 | 10.2 | 90.9 | 44.8 | 84.0 | 22.9 |
| T284A | 92.8 | 6.4 | 119.4 | 5.6 | 83.2 | 19.4 | 110.6 | 30.4 |
| H285A | 100.3 | 5.8 | 108.6 | 25.8 | 94.3 | 28.3 | 95.2 | 30.7 |
| R286A | 96.3 | 23.1 | 117.4 | 6.5 | 92.4 | 10.1 | 100.8 | 16.7 |
| R287A | 94.7 | 18.5 | 110.0 | 14.6 | 111.7 | 23.2 | 65.3 | 14.5 |
| K288A | 111.7 | 19.1 | 108.5 | 5.4 | 107.0 | 12.8 | 100.4 | 9.2 |
| A289S | 97.1 | 14.0 | 110.6 | 6.3 | 82.8 | 15.5 | 95.0 | 3.4 |
| A290S | 105.3 | 18.6 | 120.0 | 7.4 | 97.2 | 15.5 | 107.2 | 8.8 |
| R291A | 97.8 | 16.3 | 121.7 | 0.5 | 97.5 | 33.3 | 102.3 | 19.8 |
| T292A | 89.5 | 11.2 | 94.2 | 26.7 | 99.6 | 42.7 | 100.4 | 29.6 |
| A293S | 90.6 | 0.3 | 113.5 | 11.2 | 72.9 | 21.0 | 105.8 | 28.5 |
| V294A | 91.7 | 14.9 | 105.0 | 1.2 | 95.8 | 56.4 | 97.8 | 29.1 |
| G295A | 92.7 | 10.9 | 110.5 | 11.0 | 95.8 | 69.5 | 106.6 | 29.4 |
| R296A | 101.1 | 10.0 | 110.9 | 7.0 | 92.9 | 39.4 | 105.8 | 9.9 |
| N297A | 94.9 | 3.6 | 108.3 | 0.6 | 132.8 | 58.7 | 88.9 | 46.7 |
| D298A | 97.8 | 4.6 | 81.5 | 4.3 | 80.1 | 9.1 | 83.9 | 6.5 |
| T299A | 90.3 | 7.8 | 96.3 | 14.4 | 85.6 | 5.4 | 92.9 | 26.0 |
| H300A | 76.5 | 2.5 | 98.9 | 18.8 | 68.3 | 7.2 | 95.2 | 8.3 |
| P301A | 100.9 | 1.1 | 91.8 | 15.2 | 93.9 | 23.4 | 97.9 | 3.9 |
| T302A | 95.0 | 10.3 | 90.7 | 10.2 | 101.0 | 22.0 | 98.2 | 2.0 |
| T303A | 94.0 | 5.9 | 96.7 | 7.8 | 72.7 | 8.0 | 81.2 | 15.4 |
| G304A | 104.4 | 8.1 | 111.1 | 12.8 | 121.2 | 54.7 | 95.0 | 11.8 |
| S305A | 86.4 | 4.7 | 107.0 | 1.9 | 102.9 | 50.0 | 104.1 | 26.8 |
| A306S | 101.5 | 9.9 | 85.1 | 7.1 | 110.7 | 75.8 | 79.3 | 22.3 |
| S307A | 94.8 | 7.2 | 112.4 | 10.1 | 60.1 | 0.3 | 114.3 | 26.8 |
| P308A | 94.3 | 16.3 | 89.7 | 10.0 | 82.8 | 16.0 | 99.8 | 4.4 |
| K309A | 92.7 | 0.2 | 110.9 | 5.8 | 92.0 | 40.9 | 99.9 | 20.1 |
| H310A | 97.4 | 20.6 | 111.8 | 16.0 | 101.0 | 29.3 | 94.3 | 26.9 |
| Q311A | 90.4 | 3.2 | 99.5 | 32.7 | 124.2 | 60.2 | 102.4 | 17.3 |
| K312A | 83.8 | 4.5 | 110.5 | 12.3 | 99.6 | 83.5 | 98.9 | 33.5 |
| K313A | 84.1 | 4.1 | 108.5 | 1.9 | 137.0 | 30.9 | 87.7 | 7.9 |
| S314A | 96.2 | 16.8 | 95.5 | 2.7 | 138.6 | 34.3 | 95.5 | 30.1 |
| K315A | 80.6 | 20.2 | 110.9 | 18.6 | 136.3 | 88.1 | 48.4 | 25.0 |
| L316A | 84.6 | 14.5 | 103.5 | 16.2 | 90.4 | 28.9 | 80.0 | 48.5 |
| H317A | 80.8 | 2.9 | 99.8 | 13.2 | 157.1 | 17.5 | 82.6 | 5.0 |
| G318A | 98.5 | 23.2 | 105.9 | 11.2 | 117.2 | 26.0 | 92.2 | 30.3 |
| P319A | 83.9 | 17.7 | 100.2 | 7.7 | 76.4 | 16.9 | 97.0 | 18.5 |
| T320A | 98.5 | 6.1 | 111.6 | 32.9 | 82.4 | 8.2 | 108.6 | 6.5 |
| E321A | 88.3 | 11.4 | 116.6 | 28.4 | 88.4 | 17.6 | 120.3 | 35.1 |
| T322A | 104.1 | 5.0 | 111.0 | 25.5 | 83.5 | 27.1 | 98.2 | 19.5 |
| S323A | 95.2 | 3.7 | 119.3 | 22.0 | 114.9 | 16.7 | 103.2 | 5.1 |
| S324A | 97.8 | 10.8 | 106.4 | 6.9 | 108.7 | 54.1 | 96.4 | 19.8 |
| C325A | 80.1 | 5.7 | 109.3 | 4.3 | 91.2 | 28.7 | 88.6 | 22.7 |
| S326A | 85.6 | 14.7 | 109.3 | 0.6 | 129.3 | 39.2 | 65.8 | 25.7 |
| G327A | 88.0 | 13.3 | 121.8 | 25.8 | 131.8 | 43.7 | 99.1 | 33.2 |
| A328S | 86.5 | 17.9 | 102.3 | 7.6 | 127.4 | 64.6 | 75.6 | 47.4 |
| A329S | 88.1 | 14.7 | 116.1 | 16.8 | 133.0 | 34.8 | 87.3 | 38.5 |
| P330A | 98.6 | 19.5 | 119.1 | 11.8 | 145.6 | 4.0 | 97.8 | 20.6 |
| T331A | 87.4 | 11.5 | 91.3 | 6.6 | 146.7 | 41.3 | 97.9 | 35.0 |
| V332A | 106.6 | 0.3 | 91.6 | 7.4 | 89.8 | 24.1 | 93.4 | 31.6 |
| E333A | 77.9 | 11.5 | 108.3 | 4.4 | 103.5 | 14.7 | 104.0 | 13.7 |
| M334A | 94.6 | 4.5 | 106.5 | 8.7 | 81.2 | 9.1 | 110.6 | 14.5 |
| D335A | 77.8 | 4.0 | 116.8 | 2.4 | 87.4 | 10.1 | 104.9 | 20.1 |
| E336A | 82.8 | 10.8 | 117.3 | 8.0 | 103.4 | 13.2 | 108.7 | 19.1 |
| E337A | 91.1 | 2.7 | 115.6 | 20.3 | 114.0 | 5.4 | 105.9 | 14.3 |
| L338A | 83.1 | 9.8 | 106.8 | 15.6 | 89.3 | 10.2 | 109.4 | 34.8 |
| H339A | 76.7 | 9.3 | 99.5 | 11.9 | 83.5 | 5.8 | 82.0 | 8.2 |
| Y340A | 91.1 | 4.4 | 106.5 | 10.5 | 88.0 | 49.7 | 112.3 | 30.0 |
| A341S | 86.8 | 1.8 | 115.9 | 20.4 | 137.7 | 37.8 | 103.7 | 10.4 |
| S342A | 79.5 | 7.3 | 116.8 | 17.5 | 121.2 | 37.2 | 105.0 | 8.3 |
| L343A | 88.5 | 4.9 | 111.4 | 19.4 | 77.4 | 6.2 | 115.5 | 19.1 |
| N344A | 72.1 | 4.1 | 88.1 | 2.5 | 117.7 | 43.0 | 71.2 | 25.7 |
| F345A | 97.8 | 11.2 | 102.4 | 9.2 | 84.0 | 35.1 | 108.6 | 24.1 |
| H346A | 81.8 | 0.4 | 118.5 | 8.5 | 132.9 | 58.2 | 111.2 | 13.4 |
| G347A | 85.1 | 1.0 | 85.9 | 14.1 | 117.8 | 79.1 | 72.1 | 54.5 |
| M348A | 83.3 | 8.5 | 115.0 | 9.3 | 132.9 | 55.4 | 85.0 | 48.6 |
| N349A | 92.3 | 12.3 | 110.3 | 16.2 | 121.0 | 7.0 | 95.5 | 1.9 |
| P350A | 99.0 | 5.9 | 117.1 | 1.3 | 90.7 | 2.8 | 93.1 | 6.6 |
| S351A | 101.7 | 6.2 | 108.9 | 7.2 | 91.8 | 5.0 | 113.8 | 26.6 |
| K352A | 95.3 | 5.3 | 113.2 | 1.6 | 96.3 | 2.8 | 102.8 | 14.0 |
| D353A | 94.7 | 4.0 | 94.4 | 19.5 | 98.3 | 46.7 | 74.9 | 29.6 |
| T354A | 91.8 | 0.3 | 111.4 | 14.5 | 89.1 | 17.6 | 105.5 | 16.9 |
| S355A | 86.1 | 7.0 | 105.4 | 9.2 | 85.6 | 22.4 | 91.5 | 43.2 |
| T356A | 93.5 | 1.6 | 110.7 | 0.7 | 108.2 | 4.8 | 107.3 | 28.3 |
| E357A | 98.7 | 17.0 | 103.7 | 1.7 | 120.4 | 16.6 | 109.8 | 37.4 |
| Y358A | 76.1 | 5.9 | 110.4 | 6.8 | 139.7 | 70.3 | 108.6 | 26.2 |
| S359A | 95.8 | 4.0 | 101.5 | 4.6 | 134.7 | 65.2 | 82.7 | 9.8 |
| E360A | 73.7 | 38.6 | 100.8 | 14.9 | 123.1 | 38.1 | 103.9 | 20.7 |
| V361A | 88.8 | 11.1 | 99.4 | 15.1 | 97.6 | 23.4 | 92.4 | 20.0 |
| R362A | 103.9 | 20.6 | 119.1 | 11.4 | 113.5 | 14.8 | 104.3 | 4.5 |
| T363A | 91.4 | 12.9 | 107.7 | 14.3 | 91.5 | 7.3 | 123.5 | 26.0 |
| Q364A | 72.0 | 45.0 | 109.3 | 10.1 | 97.8 | 17.5 | 81.5 | 46.7 |
| | Refmab #5 | | Refmab #6 | | Refmab #7 | | Refmab #8 | |
| | Mean % | Range % | Mean % | Range % | Mean % | Range % | Mean % | Range % |
| | WT | WT | WT | WT | WT | WT | WT | WT |
| Mutation | Binding | Binding | Binding | Binding | Binding | Binding | Binding | Binding |
| D18A | 139.1 | 43.7 | 39.5 | 4.6 | 95.7 | 23.3 | 98.0 | 5.8 |
| P19A | 114.0 | 5.4 | 87.4 | 5.9 | 88.9 | 2.6 | 116.1 | 6.6 |
| N20A | 94.4 | 12.6 | 92.9 | 9.1 | 72.6 | 10.3 | 166.2 | 87.5 |
| F21A | 196.7 | 38.6 | 88.1 | 17.9 | 173.5 | 32.2 | 137.1 | 3.2 |
| W22A | 114.4 | 43.3 | 3.8 | 3.0 | 64.0 | 3.4 | 123.9 | 4.1 |
| L23A | 118.0 | 31.0 | 41.4 | 5.6 | 65.3 | 5.2 | 103.3 | 33.9 |
| Q24A | 115.0 | 36.1 | 18.1 | 0.9 | 107.5 | 9.7 | 135.9 | 25.3 |
| V25A | 143.2 | 73.0 | 16.7 | 2.4 | 84.9 | 4.5 | 113.3 | 19.1 |
| Q26A | 103.9 | | 76.1 | 13.4 | 114.1 | 21.9 | 146.6 | 6.2 |
| E27A | 186.2 | 162.6 | 92.6 | 19.8 | 111.4 | 14.0 | 108.5 | 9.7 |
| S28A | 104.6 | 31.7 | 89.4 | 11.7 | 109.6 | 16.4 | 97.9 | 14.1 |
| V29A | 121.7 | 81.5 | 112.0 | 6.7 | 123.7 | 4.3 | 107.0 | 2.4 |
| T30A | 117.6 | 17.0 | 103.5 | 21.4 | 89.3 | 7.2 | 154.0 | 54.8 |
| V31A | 112.3 | 3.9 | 105.1 | 1.6 | 92.5 | 8.7 | 124.3 | 29.4 |
| Q32A | 148.4 | 15.0 | 91.3 | 1.0 | 103.2 | 26.9 | 137.9 | 11.0 |
| E33A | 106.1 | 22.6 | 90.0 | 12.8 | 101.4 | 44.3 | 126.1 | 15.1 |
| G34A | 101.2 | 3.7 | 100.4 | 6.2 | 105.3 | 4.7 | 151.7 | 71.7 |
| L35A | 112.0 | 9.9 | 97.2 | 2.0 | 78.4 | 54.6 | 105.5 | 0.5 |
| C36A | 137.1 | 8.7 | 110.0 | 10.2 | 111.0 | 9.9 | 98.2 | 10.0 |
| V37A | 147.5 | 31.2 | 111.4 | 5.0 | 72.7 | 32.1 | 136.3 | 50.5 |
| L38A | 179.8 | 7.3 | 105.0 | 6.3 | 116.8 | 6.2 | 138.4 | 13.0 |
| V39A | 116.1 | 24.8 | 79.2 | 15.1 | 109.1 | 26.2 | 126.3 | 8.9 |
| P40A | 117.2 | 16.5 | 55.5 | 12.8 | 83.8 | 3.2 | 129.9 | 0.1 |
| C41A | 30.9 | 4.8 | 4.5 | 0.4 | 29.0 | 8.3 | 28.2 | 2.4 |
| T42A | 114.7 | 50.6 | 36.6 | 4.2 | 104.2 | 3.2 | 106.4 | 34.3 |
| F43A | 87.8 | 13.2 | 34.6 | 0.9 | 55.8 | 2.4 | 74.7 | 26.9 |
| F44A | 122.0 | 7.4 | 14.0 | 0.9 | 34.3 | 5.4 | 76.5 | 11.7 |
| H45A | 58.4 | 11.4 | 87.8 | 7.7 | 103.4 | 5.1 | 94.2 | 8.3 |
| P46A | 79.1 | 12.5 | 49.3 | 5.3 | 75.3 | 12.3 | 152.2 | 34.8 |
| I47A | 117.0 | 0.1 | 117.2 | 26.1 | 50.2 | 4.1 | 116.6 | 23.2 |
| P48A | 92.8 | 7.8 | 104.1 | 5.2 | 42.5 | 27.5 | 141.0 | 40.0 |
| Y49A | 20.6 | 19.7 | 113.4 | 0.1 | 13.7 | 0.1 | 1.7 | 7.6 |
| Y50A | 55.7 | 8.0 | 113.1 | 5.4 | 1.0 | 0.9 | 75.1 | 0.2 |
| D51A | 105.2 | 50.9 | 61.7 | 6.1 | 9.2 | 8.9 | 38.2 | 16.0 |
| K52A | 11.9 | 3.6 | 77.0 | 9.5 | 76.8 | 28.9 | 12.4 | 5.5 |
| N53A | 97.6 | 8.5 | 96.8 | 11.9 | 140.3 | 28.6 | 164.5 | 9.0 |
| S54A | 145.6 | 10.6 | 83.6 | 3.3 | 100.5 | 1.3 | 173.2 | 75.0 |
| P55A | 117.7 | 39.1 | 100.6 | 2.9 | 94.7 | 16.9 | 162.4 | 51.7 |
| V56A | 93.0 | 1.6 | 113.0 | 3.2 | 117.1 | 10.0 | 145.1 | 59.3 |
| H57A | 121.2 | 22.4 | 63.0 | 0.7 | 111.9 | 9.4 | 130.8 | 42.9 |
| G58A | 112.5 | 20.7 | 73.9 | 8.9 | 97.2 | 38.3 | 123.0 | 9.0 |
| Y59A | 205.0 | 95.8 | 83.2 | 14.9 | 127.3 | 6.6 | 125.1 | 22.7 |
| W60A | 35.9 | 18.0 | 2.1 | 1.3 | 2.1 | 0.9 | 9.6 | 13.7 |
| F61A | 161.4 | 79.7 | 110.6 | 0.2 | 123.4 | 18.6 | 124.7 | 22.1 |
| R62A | 123.3 | 1.7 | 105.8 | 0.2 | 114.3 | 25.7 | 162.3 | 15.4 |
| E63A | 139.5 | 6.0 | 90.9 | 8.2 | 93.0 | 22.3 | 153.1 | 25.3 |
| G64A | 135.9 | 6.5 | 92.9 | 1.4 | 118.6 | 23.3 | 149.2 | 17.6 |
| A65S | 174.7 | 61.8 | 92.3 | 9.0 | 116.6 | 12.9 | 153.1 | 6.4 |
| I66A | 104.7 | 7.8 | 93.7 | 2.0 | 117.0 | 4.7 | 144.1 | 50.9 |
| I67A | 156.6 | 14.8 | 108.2 | 15.1 | 134.7 | 37.3 | 179.6 | 4.8 |
| S68A | 142.2 | 47.7 | 86.4 | 5.6 | 119.8 | 12.6 | 177.2 | 30.3 |
| R69A | 164.0 | 10.6 | 100.6 | 22.5 | 146.2 | 29.7 | 170.2 | 31.6 |
| D70A | 111.5 | 21.8 | 80.3 | 2.2 | 81.2 | 29.8 | 137.2 | 19.2 |
| S71A | 149.6 | 57.8 | 99.4 | 13.8 | 103.8 | 13.3 | 141.2 | 36.9 |
| P72A | 116.2 | 13.2 | 92.3 | 7.8 | 114.0 | 2.2 | 181.0 | 64.4 |
| V73A | 111.9 | 39.2 | 77.7 | 4.1 | 113.2 | 26.0 | 132.3 | 36.6 |
| A74S | 99.0 | 48.6 | 94.9 | 13.7 | 117.7 | 18.6 | 119.8 | 25.2 |
| T75A | 120.1 | 2.5 | 102.4 | 0.1 | 115.0 | 9.9 | 123.8 | 9.0 |
| N76A | 124.5 | 32.8 | 86.8 | 3.1 | 93.9 | 7.4 | 127.6 | 14.3 |
| K77A | 124.5 | 11.8 | 79.9 | 31.5 | 117.4 | 25.0 | 152.0 | 9.9 |
| L78A | 99.3 | 15.9 | 110.3 | 7.9 | 88.9 | 13.2 | 175.8 | 5.7 |
| D79A | 91.3 | 4.2 | 78.2 | 7.2 | 83.5 | 8.2 | 129.5 | 39.0 |
| Q80A | 150.8 | 17.8 | 101.5 | 3.9 | 123.8 | 25.4 | 143.3 | 6.2 |
| E81A | 140.4 | 1.4 | 88.1 | 6.6 | 98.4 | 12.0 | 134.6 | 15.2 |
| V82A | 121.9 | 38.4 | 110.1 | 20.0 | 108.5 | 23.4 | 113.8 | 2.7 |
| Q83A | 97.0 | 21.7 | 107.8 | 5.5 | 119.9 | 19.0 | 127.1 | 11.4 |
| E84A | 140.7 | 8.7 | 97.4 | 5.5 | 108.8 | 30.2 | 137.5 | 21.3 |
| E85A | 157.7 | 61.3 | 94.4 | 3.4 | 126.1 | 22.9 | 148.4 | 4.1 |
| T86A | 123.6 | 13.6 | 80.1 | 0.4 | 110.7 | 27.4 | 165.4 | 53.5 |
| Q87A | 137.1 | 65.7 | 96.9 | 17.9 | 90.1 | 9.6 | 124.8 | 25.9 |
| G88A | 118.0 | 32.3 | 104.9 | 14.0 | 115.2 | 19.2 | 111.5 | 11.6 |
| R89A | 137.2 | 63.6 | 80.8 | 12.3 | 94.0 | 11.5 | 125.5 | 25.7 |
| F90A | 122.8 | 30.4 | 71.1 | 11.3 | 83.3 | 34.8 | 101.7 | 13.8 |
| R91A | 101.3 | 7.2 | 91.4 | 0.6 | 114.5 | 36.6 | 127.3 | 34.9 |
| L92A | 126.8 | 20.7 | 85.3 | 8.9 | 89.9 | 0.4 | 119.1 | 23.9 |
| L93A | 134.3 | 38.3 | 94.2 | 5.7 | 101.5 | 8.2 | 118.1 | 43.1 |
| G94A | 92.2 | 36.8 | 93.1 | 4.0 | 99.2 | 0.5 | 115.4 | 32.8 |
| D95A | 122.2 | 11.8 | 86.3 | 6.6 | 103.8 | 29.8 | 139.8 | 5.1 |
| P96A | 114.2 | 29.0 | 92.3 | 8.2 | 98.7 | 20.9 | 108.6 | 4.4 |
| S97A | 126.1 | 16.4 | 93.7 | 5.7 | 128.1 | 20.0 | 146.7 | 7.8 |
| R98A | 134.3 | 5.1 | 94.2 | 7.6 | 137.8 | 24.7 | 164.9 | 18.4 |
| N99A | 88.4 | 20.6 | 92.0 | 15.5 | 102.0 | 6.4 | 123.9 | 16.0 |
| N100A | 165.2 | 86.6 | 83.9 | 19.3 | 127.6 | 35.5 | 165.2 | 24.6 |
| C101A | 23.0 | 12.7 | 6.2 | 9.4 | 1.8 | 1.3 | 5.3 | 0.8 |
| S102A | 153.6 | 17.6 | 85.1 | 10.5 | 111.2 | 25.2 | 145.4 | 23.7 |
| L103A | 91.6 | 38.9 | 61.5 | 2.8 | 108.7 | 8.0 | 134.4 | 9.8 |
| S104A | 108.7 | 22.8 | 90.9 | 5.0 | 117.3 | 27.6 | 121.8 | 12.3 |
| I105A | 41.7 | 2.8 | 25.5 | 1.3 | 56.1 | 4.9 | 82.9 | 13.0 |
| V106A | 105.8 | 24.4 | 125.3 | 11.0 | 126.5 | 22.5 | 110.0 | 20.6 |
| D107A | 118.9 | 28.0 | 96.5 | 7.9 | 113.1 | 2.6 | 129.1 | 17.7 |
| A108S | 105.4 | 2.0 | 78.4 | 23.2 | 119.6 | 46.2 | 120.9 | 23.4 |
| R109A | 102.9 | 16.0 | 106.8 | 3.2 | 126.9 | 27.4 | 131.9 | 53.1 |
| R110A | 125.1 | 17.3 | 98.4 | 12.4 | 95.9 | 2.3 | 115.2 | 10.8 |
| R111A | 117.7 | 18.0 | 102.1 | 2.9 | 94.0 | 7.0 | 128.6 | 47.5 |
| D112A | 61.2 | 1.8 | 51.6 | 14.2 | 47.9 | 1.2 | 75.8 | 0.0 |
| N113A | 64.0 | 23.4 | 89.7 | 3.5 | 80.0 | 4.2 | 122.3 | 36.2 |
| G114A | 79.0 | 7.3 | 105.5 | 3.9 | 94.6 | 7.0 | 94.8 | 22.7 |
| S115A | 74.6 | 6.2 | 75.8 | 5.6 | 95.1 | 2.6 | 134.2 | 54.4 |
| Y116A | 16.0 | 1.0 | 5.0 | 2.9 | 1.7 | 1.0 | 3.7 | 5.7 |
| F117A | 112.8 | 23.2 | 90.3 | 14.0 | 131.6 | 9.3 | 121.9 | 0.1 |
| F118A | 11.1 | 0.4 | 4.9 | 0.3 | 3.2 | 4.7 | 8.7 | 15.6 |
| R119A | 167.0 | 19.8 | 123.4 | 17.0 | 116.1 | 18.4 | 124.5 | 37.3 |
| M120A | 73.2 | 20.1 | 76.3 | 32.3 | 92.0 | 1.3 | 114.3 | 19.2 |
| E121A | 104.9 | 16.2 | 90.8 | 0.1 | 94.4 | 5.9 | 117.8 | 37.5 |
| R122A | 85.7 | 10.5 | 105.8 | 1.8 | 25.1 | 8.0 | 99.0 | 8.3 |
| G123A | 67.5 | 7.1 | 94.1 | 19.2 | 76.0 | 20.2 | 128.0 | 10.9 |
| S124A | 79.4 | 13.5 | 98.2 | 14.7 | 81.8 | 3.8 | 91.0 | 17.0 |
| T125A | 56.7 | | 111.5 | 35.4 | 91.3 | 6.2 | 90.2 | 6.6 |
| K126A | 133.9 | 39.5 | 133.6 | 15.3 | 156.3 | 28.6 | 132.6 | 17.2 |
| Y127A | 199.1 | 8.6 | 42.0 | 6.6 | 174.8 | 3.6 | 109.6 | 29.8 |
| S128A | 282.8 | 99.0 | 127.3 | 8.7 | 178.7 | 7.9 | 136.7 | 13.9 |
| Y129A | 196.3 | | 76.7 | 5.0 | 155.4 | 22.9 | 108.3 | |
| K130A | 270.9 | 61.4 | 118.8 | 13.2 | 148.2 | 4.0 | 128.4 | 15.6 |
| S131A | 106.5 | 39.1 | 10.8 | 1.9 | 114.0 | 8.5 | 115.4 | 40.3 |
| P132A | 97.3 | 11.4 | 3.6 | 1.4 | 103.9 | 0.1 | 150.1 | 23.8 |
| Q133A | 112.5 | 16.0 | 88.3 | 5.2 | 100.3 | 9.2 | 133.9 | 17.2 |
| L134A | 95.9 | 30.5 | 19.5 | 3.2 | 96.6 | 11.9 | 93.9 | 13.6 |
| S135A | 108.5 | 29.7 | 110.4 | 2.2 | 101.1 | 12.7 | 133.1 | 7.6 |
| V136A | 104.5 | 10.2 | 65.8 | 4.0 | 85.9 | 13.0 | 112.7 | 4.2 |
| H137A | 120.0 | 15.6 | 117.3 | 9.1 | 113.9 | 9.0 | 92.6 | 19.9 |
| V138A | 101.1 | 20.2 | 65.4 | 1.4 | 91.0 | 6.4 | 126.2 | 36.3 |
| T139A | 103.5 | 13.6 | 97.7 | 20.2 | 120.6 | 6.5 | 138.6 | 4.7 |
| D140A | 118.1 | 10.0 | 104.2 | 11.0 | 115.9 | 1.6 | 134.4 | 10.9 |
| L141A | 94.5 | 36.3 | 97.5 | 7.0 | 118.2 | 13.4 | 102.7 | 56.4 |
| T142A | 101.4 | 43.2 | 91.6 | 3.7 | 112.9 | 12.4 | 120.5 | 48.1 |
| H143A | 128.1 | 2.8 | 97.5 | 1.8 | 112.5 | 33.3 | 137.7 | 18.4 |
| R144A | 71.0 | 12.5 | 103.7 | 3.8 | 120.3 | 16.3 | 109.5 | 29.6 |
| P145A | 82.9 | 8.4 | 89.7 | 6.5 | 102.1 | 17.4 | 142.6 | 1.0 |
| K146A | 148.6 | 24.4 | 116.4 | 19.6 | 91.1 | 9.1 | 135.1 | 10.1 |
| I147A | 77.2 | 37.0 | 65.4 | 5.0 | 92.0 | 11.4 | 159.1 | 16.6 |
| L148A | 112.6 | 1.1 | 93.3 | 1.8 | 96.7 | 8.5 | 145.4 | 15.7 |
| I149A | 112.4 | 7.8 | 100.0 | 13.6 | 96.9 | 14.0 | 143.9 | 64.6 |
| P150A | 131.6 | 5.7 | 105.8 | 6.3 | 114.6 | 2.0 | 136.1 | 15.8 |
| G151A | 99.6 | 3.5 | 91.1 | 11.6 | 120.7 | 0.6 | 139.1 | 37.3 |
| T152A | 137.0 | 12.4 | 92.7 | 0.1 | 102.0 | 3.6 | 152.6 | 60.4 |
| L153A | 100.8 | 57.7 | 94.3 | 11.6 | 101.0 | 4.1 | 155.2 | 29.4 |
| E154A | 112.5 | 8.7 | 86.2 | 21.6 | 108.1 | 6.8 | 129.5 | 16.3 |
| P155A | 140.5 | 21.9 | 104.2 | 5.3 | 107.3 | 0.9 | 125.1 | 28.0 |
| G156A | 92.3 | 20.2 | 88.1 | 1.5 | 112.1 | 10.0 | 115.5 | 22.2 |
| H157A | 102.1 | 29.2 | 96.3 | 13.0 | 116.3 | 0.9 | 105.7 | 18.8 |
| S158A | 99.2 | 18.2 | 95.2 | 1.9 | 112.7 | 2.9 | 123.7 | 3.3 |
| K159A | 147.9 | 18.5 | 122.7 | 7.9 | 109.9 | 10.6 | 164.2 | 14.4 |
| N160A | 109.6 | 18.9 | 106.4 | 7.2 | 112.0 | 18.5 | 138.4 | 52.5 |
| L161A | 129.5 | 14.7 | 100.0 | 8.1 | 120.2 | 9.3 | 156.1 | 1.6 |
| T162A | 92.4 | 19.1 | 87.2 | 7.2 | 103.4 | 16.4 | 134.6 | 3.2 |
| C163A | 62.0 | 11.3 | 73.9 | 0.3 | 102.1 | 0.4 | 134.3 | 27.7 |
| S164A | 109.8 | 11.6 | 100.3 | 9.0 | 122.6 | 5.4 | 141.0 | 19.6 |
| V165A | 173.6 | 16.4 | 100.9 | 10.7 | 117.9 | 13.3 | 173.2 | 36.1 |
| S166A | 114.5 | 39.1 | 108.0 | 1.9 | 107.6 | 11.4 | 186.0 | 49.3 |
| W167A | 95.5 | 13.6 | 90.5 | 7.1 | 127.2 | 12.8 | 134.4 | 26.0 |
| A168S | 102.6 | 8.4 | 100.1 | 9.4 | 117.2 | 10.9 | 129.7 | 15.9 |
| C169A | 101.2 | 0.9 | 76.2 | 11.2 | 90.0 | 18.1 | 131.0 | 30.1 |
| E170A | 110.1 | 13.5 | 89.5 | 7.4 | 117.8 | 5.2 | 127.9 | 8.5 |
| Q171A | 105.2 | 22.3 | 99.6 | 10.4 | 120.6 | 12.3 | 136.2 | 4.6 |
| G172A | 92.7 | 14.6 | 76.8 | 7.6 | 92.1 | 2.6 | 150.3 | 7.6 |
| T173A | 100.7 | 9.0 | 98.9 | 0.3 | 103.2 | 12.0 | 148.5 | 46.7 |
| P174A | 118.1 | 5.9 | 103.0 | 13.4 | 122.4 | 5.5 | 154.6 | 18.1 |
| P175A | 129.9 | 16.3 | 98.6 | 1.1 | 126.2 | 12.0 | 130.7 | 24.5 |
| I176A | 97.0 | 38.6 | 102.3 | 1.7 | 102.6 | 16.9 | 174.2 | 33.0 |
| F177A | 57.3 | 7.7 | 48.7 | 11.2 | 74.1 | 18.0 | 131.6 | 26.4 |
| S178A | 117.1 | 43.0 | 101.7 | 19.3 | 105.7 | 18.9 | 163.0 | 7.1 |
| W179A | 132.8 | 16.0 | 96.7 | 14.9 | 111.3 | 25.2 | 135.3 | 31.6 |
| L180A | 118.3 | 9.3 | 97.1 | 12.3 | 104.9 | 17.9 | 126.4 | 19.8 |
| S181A | 127.6 | 24.3 | 102.5 | 3.8 | 86.7 | 9.8 | 125.9 | 19.5 |
| A182S | 103.2 | 6.7 | 93.9 | 6.6 | 113.8 | 19.1 | 121.5 | 3.8 |
| A183S | 101.3 | 11.2 | 92.3 | 7.8 | 101.6 | 11.7 | 110.1 | 56.1 |
| P184A | 106.7 | 10.6 | 92.2 | 0.5 | 105.5 | 19.7 | 128.6 | 16.2 |
| T185A | 94.0 | 15.3 | 116.9 | 21.8 | 106.1 | 2.3 | 111.5 | 39.1 |
| S186A | 111.8 | 20.1 | 109.7 | 6.5 | 93.4 | 8.2 | 144.0 | 11.8 |
| L187A | 143.9 | 21.9 | 119.3 | 1.4 | 124.1 | 1.1 | 148.8 | 93.1 |
| G188A | 114.0 | 11.1 | 100.5 | 8.8 | 127.3 | 13.1 | 190.0 | 18.0 |
| P189A | 114.6 | 3.0 | 103.0 | 17.9 | 118.6 | 8.2 | 159.4 | 7.2 |
| R190A | 111.8 | 30.1 | 111.8 | 9.2 | 123.2 | 2.8 | 160.0 | 28.1 |
| T191A | 114.6 | 29.6 | 88.5 | 7.5 | 116.2 | 18.5 | 151.9 | 20.9 |
| T192A | 109.0 | 6.9 | 99.0 | 11.7 | 102.5 | 0.5 | 146.2 | 8.6 |
| H193A | 119.4 | 18.8 | 100.4 | 24.8 | 107.3 | 2.3 | 140.2 | 47.5 |
| S194A | 90.4 | 15.4 | 92.4 | 6.6 | 111.7 | 8.1 | 153.8 | 11.1 |
| S195A | 117.2 | 5.0 | 113.4 | 7.7 | 117.2 | 6.7 | 147.6 | 0.5 |
| V196A | 84.5 | 4.8 | 116.5 | 10.2 | 117.3 | 3.8 | 110.4 | 17.2 |
| L197A | 102.4 | 31.4 | 103.2 | 2.9 | 115.4 | 0.1 | 105.0 | 0.1 |
| —, I198A | 84.7 | 3.8 | 89.3 | 11.5 | 110.9 | 6.5 | 134.1 | 75.8 |
| I199A | 131.8 | 23.1 | 106.5 | 3.5 | 114.7 | 25.9 | 113.8 | 35.7 |
| T200A | 98.3 | 32.9 | 109.7 | 10.0 | 97.6 | 20.9 | 116.7 | 20.7 |
| P201A | 111.4 | 24.3 | 115.2 | 10.0 | 109.8 | 29.7 | 113.9 | 19.9 |
| R202A | 123.0 | 26.0 | 92.2 | 23.4 | 104.5 | 9.6 | 128.6 | 23.6 |
| P203A | 104.8 | 21.3 | 102.9 | 11.3 | 113.4 | 3.7 | 146.5 | 32.1 |
| Q204A | 102.8 | 26.2 | 107.5 | 3.2 | 117.3 | 24.3 | 168.0 | 59.5 |
| D205A | 12.3 | 10.3 | 3.1 | 2.4 | 0.8 | 0.5 | 1.8 | 2.3 |
| H206A | 108.2 | 26.6 | 98.3 | 2.9 | 108.3 | 22.8 | 98.0 | 9.2 |
| G207A | 108.7 | 34.8 | 98.6 | 0.4 | 103.9 | 16.4 | 143.2 | 45.8 |
| T208A | 100.0 | 7.7 | 116.4 | 5.4 | 101.8 | 5.6 | 123.6 | 20.0 |
| N209A | 88.1 | 1.8 | 100.8 | 0.4 | 127.9 | 3.7 | 115.7 | 7.1 |
| L210A | 78.2 | 2.7 | 96.9 | 7.9 | 98.6 | 31.6 | 118.5 | 33.1 |
| T211A | 80.4 | 7.8 | 92.2 | 11.2 | 98.1 | 38.6 | 127.0 | 24.4 |
| C212A | 28.4 | 6.4 | 16.2 | 1.0 | 17.8 | 9.3 | 51.4 | 9.2 |
| Q213A | 127.5 | 29.0 | 98.0 | 17.0 | 102.7 | 5.5 | 138.5 | 26.0 |
| V214A | 127.3 | 3.7 | 94.4 | 11.1 | 119.7 | 29.4 | 141.6 | 61.3 |
| K215A | 102.8 | 30.0 | 85.6 | 9.0 | 144.7 | 69.1 | 145.6 | 42.2 |
| F216A | 103.9 | 8.4 | 82.5 | 5.0 | 111.5 | 7.0 | 120.5 | 1.3 |
| A217S | 121.0 | 17.7 | 91.0 | 22.0 | 113.7 | 5.5 | 101.6 | 23.4 |
| G218A | 101.8 | | 98.0 | 25.1 | 122.8 | 11.5 | 114.5 | 31.4 |
| A219S | 93.7 | 27.7 | 92.0 | 7.8 | 121.2 | 21.9 | 118.9 | 39.5 |
| G220A | 93.5 | 1.3 | 99.8 | 3.2 | 120.4 | 9.3 | 117.7 | 0.5 |
| V221A | 116.5 | 27.6 | 94.6 | 10.2 | 99.7 | 16.1 | 133.6 | 25.3 |
| T222A | 111.3 | 21.6 | 92.2 | 0.5 | 109.9 | 30.6 | 127.0 | 16.6 |
| T223A | 114.6 | 18.2 | 109.9 | 6.6 | 110.0 | 14.9 | 145.2 | 15.3 |
| E224A | 97.1 | 10.1 | 106.0 | 4.2 | 125.6 | 25.5 | 130.2 | 2.1 |
| R225A | 123.8 | 29.2 | 103.5 | 2.8 | 99.8 | 7.4 | 142.6 | 21.8 |
| T226A | 97.9 | 11.2 | 96.5 | 10.6 | 99.1 | 8.7 | 133.5 | 10.3 |
| I227A | 105.2 | 3.4 | 88.1 | 6.3 | 101.2 | 2.2 | 141.0 | 22.6 |
| Q228A | 129.0 | 39.6 | 100.2 | 7.6 | 114.9 | 16.9 | 148.2 | 22.1 |
| L229A | 106.2 | 46.0 | 97.1 | 0.1 | 114.1 | 6.9 | 161.0 | 19.4 |
| N230A | 105.1 | 11.0 | 83.8 | 7.3 | 100.0 | 7.4 | 147.3 | 12.4 |
| V231A | 88.2 | 48.8 | 92.8 | 14.4 | 112.3 | 14.2 | 153.3 | 9.4 |
| T232A | 81.5 | 6.8 | 79.6 | 3.6 | 95.3 | 4.0 | 105.4 | 17.6 |
| Y233A | 89.5 | 3.4 | 103.2 | 2.0 | 108.8 | 9.5 | 105.0 | 29.8 |
| V234A | 115.0 | 4.0 | 109.3 | 23.1 | 98.6 | 39.9 | 145.5 | 37.4 |
| P235A | 102.1 | 16.9 | 84.3 | 13.4 | 111.5 | 1.6 | 104.4 | 0.4 |
| Q236A | 100.2 | 24.1 | 102.8 | 0.5 | 94.3 | 16.4 | 168.8 | 47.8 |
| N237A | 91.8 | 12.2 | 100.6 | 13.2 | 116.1 | 7.2 | 142.4 | 24.9 |
| P238A | 157.1 | 16.4 | 113.8 | 3.7 | 128.6 | 4.3 | 128.7 | 17.5 |
| T239A | 106.9 | 23.8 | 87.3 | 16.6 | 111.5 | 7.3 | 146.2 | 17.2 |
| T240A | 96.9 | 21.2 | 97.0 | 4.5 | 120.8 | 4.6 | 131.6 | 47.5 |
| G241A | 108.9 | 33.7 | 101.4 | 1.0 | 131.3 | 17.8 | 140.3 | 3.2 |
| I242A | 81.8 | 1.2 | 99.0 | 2.2 | 105.3 | 19.7 | 129.1 | 28.2 |
| F243A | 102.8 | 39.6 | 98.2 | 7.1 | 91.8 | 8.8 | 181.3 | 11.8 |
| P244A | 107.2 | 47.7 | 101.1 | 3.4 | 99.0 | 2.6 | 192.7 | 21.6 |
| G245A | 109.8 | 54.5 | 91.8 | 17.3 | 122.5 | 6.9 | 139.6 | 23.7 |
| D246A | 114.9 | 7.9 | 105.5 | 20.1 | 99.5 | 3.1 | 150.7 | 45.2 |
| G247A | 85.6 | 4.5 | 105.2 | 15.9 | 122.7 | 15.3 | 127.4 | 55.1 |
| S248A | 104.6 | 37.7 | 94.4 | 0.0 | 112.8 | 3.6 | 136.5 | 37.1 |
| G249A | 105.0 | 54.4 | 96.6 | 23.1 | 113.9 | 13.9 | 132.0 | 18.7 |
| K250A | 85.7 | 20.9 | 98.0 | 5.7 | 118.5 | 4.0 | 128.6 | 30.0 |
| Q251A | 94.7 | 16.4 | 126.5 | 18.4 | 107.8 | 10.8 | 207.8 | 38.1 |
| E252A | 114.3 | 7.3 | 108.3 | 6.0 | 120.2 | 7.0 | 144.2 | 12.1 |
| T253A | 94.6 | 43.2 | 96.8 | 0.5 | 111.7 | 9.2 | 170.3 | 0.4 |
| R254A | 94.7 | 20.3 | 95.7 | 1.3 | 126.3 | 10.9 | 140.8 | 0.0 |
| A255S | 88.3 | 17.0 | 96.8 | 1.7 | 120.8 | 3.2 | 142.3 | 19.6 |
| G256A | 87.6 | 14.6 | 107.9 | 3.1 | 105.7 | 9.1 | 141.8 | 69.3 |
| V257A | 133.9 | 16.8 | 98.1 | 4.6 | 123.5 | 7.0 | 156.1 | 8.3 |
| V258A | 95.9 | 36.4 | 95.9 | 4.9 | 98.8 | 18.5 | 160.6 | 56.5 |
| H259A | 94.5 | 26.0 | 86.4 | 20.3 | 118.7 | 11.2 | 143.4 | 8.4 |
| G260A | 102.0 | 4.6 | 97.2 | 0.4 | 87.2 | 19.7 | 147.2 | 47.2 |
| A261S | 79.8 | 5.5 | 56.7 | 35.4 | 102.2 | 5.8 | 145.8 | 8.6 |
| I262A | 97.5 | 15.8 | 99.4 | 8.2 | 119.0 | 5.8 | 157.2 | 7.2 |
| G263A | 103.7 | 26.9 | 94.3 | 1.2 | 104.0 | 37.0 | 142.7 | 4.2 |
| G264A | 94.4 | 17.4 | 103.1 | 5.9 | 82.8 | 19.1 | 134.8 | 3.7 |
| A265S | 99.7 | 12.7 | 107.3 | 20.3 | 109.7 | 7.0 | 155.9 | 4.7 |
| G266A | 111.1 | 50.4 | 82.6 | 30.4 | 110.3 | 10.4 | 150.5 | 1.2 |
| V267A | 95.2 | 1.6 | 101.4 | 1.5 | 92.6 | 3.8 | 149.0 | 18.3 |
| T268A | 84.7 | 7.9 | 100.5 | 0.9 | 148.5 | 45.9 | 136.0 | 8.4 |
| A269S | 123.9 | 9.8 | 93.9 | 15.1 | 118.0 | 26.1 | 156.6 | 7.2 |
| L270A | 91.0 | 4.4 | 104.7 | 13.7 | 99.8 | 22.5 | 135.6 | 31.3 |
| L271A | 84.9 | 21.5 | 105.8 | 9.7 | 113.3 | 19.3 | 118.1 | 22.1 |
| A272S | 109.6 | 6.9 | 90.9 | 22.4 | 113.3 | 6.2 | 158.0 | 4.3 |
| L273A | 101.5 | 43.8 | 103.9 | 15.6 | 92.5 | 24.1 | 178.3 | 26.5 |
| C274A | 87.4 | 16.2 | 92.3 | 0.3 | 102.5 | 14.3 | 146.0 | 31.6 |
| L275A | 86.4 | 5.8 | 94.6 | 13.0 | 106.4 | 5.4 | 130.0 | 45.7 |
| C276A | 95.4 | 15.0 | 100.4 | 2.7 | 110.2 | 25.7 | 130.4 | 8.0 |
| L277A | 89.0 | 10.2 | 103.6 | 6.1 | 92.0 | 19.3 | 105.5 | 24.7 |
| I278A | 100.0 | 11.7 | 107.6 | 1.6 | 93.2 | 0.6 | 138.1 | 24.7 |
| F279A | 100.3 | 13.2 | 101.8 | 3.7 | 123.7 | 23.4 | 117.4 | 37.8 |
| F280A | 123.8 | 7.2 | 105.9 | 10.6 | 104.5 | 3.6 | 99.5 | 7.6 |
| I281A | 115.0 | 2.5 | 103.7 | 1.7 | 109.5 | 7.4 | 115.6 | 33.1 |
| V282A | 112.1 | 0.2 | 107.7 | 7.5 | 105.6 | 18.4 | 146.5 | 24.3 |
| K283A | 82.9 | 1.3 | 88.6 | 6.2 | 117.2 | 30.5 | 109.2 | 20.2 |
| T284A | 103.1 | 7.5 | 104.6 | 11.2 | 120.5 | 4.5 | 150.2 | 7.3 |
| H285A | 115.9 | 12.7 | 102.8 | 1.0 | 109.4 | 32.4 | 152.3 | 15.8 |
| R286A | 97.5 | 5.2 | 95.6 | 2.0 | 134.2 | 38.9 | 126.4 | 6.3 |
| R287A | 97.1 | 17.7 | 71.5 | 9.8 | 91.1 | 30.5 | 147.8 | 19.9 |
| K288A | 97.9 | 3.1 | 100.1 | 3.4 | 127.7 | 11.9 | 148.3 | 11.1 |
| A289S | 91.8 | 24.3 | 96.0 | 8.1 | 107.1 | 18.1 | 117.6 | 57.2 |
| A290S | 118.4 | 32.6 | 107.9 | 1.3 | 98.0 | 0.2 | 133.7 | 20.1 |
| R291A | 103.1 | 12.8 | 104.2 | 12.8 | 119.5 | 23.2 | 161.7 | 38.9 |
| T292A | 85.1 | 3.9 | 112.6 | 13.8 | 118.9 | 10.7 | 121.7 | 32.6 |
| A293S | 87.7 | 5.3 | 101.3 | 3.7 | 120.4 | 10.2 | 107.0 | 13.1 |
| V294A | 84.6 | 3.9 | 103.7 | 1.8 | 126.1 | 1.0 | 126.8 | 4.8 |
| G295A | 100.7 | 15.8 | 92.1 | 16.1 | 115.3 | 3.1 | 149.3 | 13.7 |
| R296A | 99.5 | 8.3 | 101.8 | 4.4 | 93.6 | 32.8 | 108.6 | 27.4 |
| N297A | 83.7 | 20.3 | 95.6 | 6.6 | 81.2 | 15.4 | 151.3 | 1.6 |
| D298A | 84.5 | 2.9 | 106.3 | 9.2 | 86.5 | 5.6 | 117.6 | 16.0 |
| T299A | 91.1 | 31.7 | 104.0 | 1.8 | 109.0 | 13.2 | 114.8 | 9.8 |
| H300A | 92.8 | 4.6 | 101.2 | 1.7 | 106.5 | 25.8 | 126.0 | 27.4 |
| P301A | 90.1 | 4.3 | 104.9 | 9.3 | 99.8 | 9.1 | 124.2 | 47.5 |
| T302A | 77.0 | 11.6 | 100.8 | 4.7 | 112.7 | 14.4 | 125.7 | 6.0 |
| T303A | 82.4 | 2.4 | 95.5 | 3.8 | 77.1 | 22.5 | 121.3 | 16.8 |
| G304A | 79.1 | 3.4 | 100.1 | 3.9 | 102.6 | 17.6 | 96.6 | 48.8 |
| S305A | 103.3 | 7.2 | 86.8 | 4.2 | 106.2 | 4.6 | 144.2 | 5.0 |
| A306S | 88.5 | 8.9 | 99.2 | 19.1 | 100.2 | 11.7 | 124.8 | 33.7 |
| S307A | 83.0 | 8.5 | 116.1 | 2.1 | 91.7 | 2.0 | 131.6 | 12.7 |
| P308A | 73.5 | 11.7 | 103.1 | 3.7 | 111.3 | 39.2 | 104.7 | 8.8 |
| K309A | 85.7 | 4.0 | 98.8 | 16.9 | 93.6 | 32.6 | 148.5 | 11.7 |
| H310A | 91.3 | 9.3 | 94.9 | 6.5 | 90.4 | 24.4 | 99.6 | 19.4 |
| Q311A | 125.7 | 57.5 | 88.7 | 10.1 | 93.6 | 2.1 | 110.3 | 17.3 |
| K312A | 94.7 | 10.1 | 94.5 | 6.5 | 101.1 | 17.7 | 133.1 | 24.5 |
| K313A | 117.6 | 16.7 | 101.9 | 0.6 | 107.9 | 3.1 | 121.9 | 19.6 |
| S314A | 112.4 | 17.6 | 96.3 | 7.7 | 106.7 | 6.7 | 115.0 | 9.2 |
| K315A | 112.5 | 39.7 | 99.2 | 1.0 | 112.0 | 8.1 | 131.1 | 60.5 |
| L316A | 79.5 | 27.3 | 90.5 | 18.9 | 103.3 | 19.0 | 131.4 | 14.3 |
| H317A | 71.9 | 12.9 | 95.2 | 21.3 | 112.2 | 0.3 | 98.5 | 7.3 |
| G318A | 87.1 | 4.1 | 89.1 | 7.3 | 128.2 | 10.8 | 100.7 | 18.4 |
| P319A | 70.0 | 12.3 | 85.2 | 9.6 | 113.5 | 15.2 | 124.3 | 14.5 |
| T320A | 120.8 | 3.9 | 100.1 | 8.6 | 117.2 | 24.5 | 133.4 | 25.2 |
| E321A | 96.2 | 11.0 | 110.7 | 1.5 | 109.0 | 11.1 | 125.7 | 5.3 |
| T322A | 100.4 | 1.2 | 110.3 | 3.1 | 101.8 | 9.8 | 136.4 | 38.3 |
| S323A | 113.2 | 24.2 | 105.9 | 0.8 | 108.7 | 25.0 | 135.6 | 18.1 |
| S324A | 96.6 | 32.2 | 97.1 | 18.6 | 99.2 | 12.3 | 146.4 | 54.6 |
| C325A | 106.9 | 11.1 | 110.7 | 13.2 | 101.1 | 11.3 | 102.5 | 35.8 |
| S326A | 106.8 | 7.4 | 88.8 | 34.4 | 102.8 | 17.3 | 134.5 | 14.2 |
| G327A | 98.7 | 40.8 | 92.1 | 9.1 | 109.5 | 12.1 | 158.0 | 18.4 |
| A328S | 104.9 | 3.2 | 94.0 | 16.2 | 119.4 | 16.1 | 140.6 | 15.5 |
| A329S | 124.5 | 0.7 | 92.8 | 12.2 | 119.7 | 7.4 | 120.7 | 9.7 |
| P330A | 90.3 | 47.3 | 97.9 | 0.8 | 101.6 | 33.1 | 135.2 | 20.2 |
| T331A | 88.1 | 30.7 | 90.9 | 6.7 | 108.5 | 5.7 | 104.8 | 45.2 |
| V332A | 71.6 | 2.4 | 100.3 | 1.4 | 111.9 | 14.0 | 124.6 | 5.1 |
| E333A | 121.3 | 44.6 | 98.7 | 0.6 | 91.0 | 5.1 | 137.5 | 15.0 |
| M334A | 99.4 | 17.7 | 107.2 | 2.9 | 93.9 | 9.5 | 143.4 | 12.1 |
| D335A | 86.2 | 6.1 | 98.2 | 9.6 | 106.5 | 3.6 | 137.5 | 33.5 |
| E336A | 90.5 | 20.1 | 101.9 | 0.5 | 98.4 | 12.0 | 129.7 | 40.2 |
| E337A | 114.2 | 12.0 | 104.8 | 4.9 | 101.5 | 8.0 | 128.4 | 40.0 |
| L338A | 104.7 | 9.0 | 102.6 | 7.7 | 124.5 | 48.4 | 161.6 | 6.5 |
| H339A | 82.7 | 4.3 | 99.3 | 4.9 | 132.9 | 56.0 | 127.3 | 21.0 |
| Y340A | 88.6 | 2.7 | 101.3 | 3.3 | 112.0 | 0.3 | 159.2 | 18.2 |
| A341S | 112.8 | 24.1 | 89.6 | 9.7 | 95.0 | 12.6 | 157.5 | 10.7 |
| S342A | 101.1 | 29.9 | 102.2 | 6.8 | 95.2 | 0.2 | 159.0 | 4.6 |
| L343A | 77.8 | 17.5 | 101.4 | 6.6 | 96.2 | 13.9 | 141.5 | 34.2 |
| N344A | 87.9 | 19.6 | 67.7 | 4.8 | 108.4 | 14.0 | 133.4 | 16.3 |
| F345A | 86.4 | 2.9 | 98.4 | 9.6 | 139.5 | 37.0 | 163.0 | 4.9 |
| H346A | 113.6 | 66.1 | 103.8 | 5.3 | 98.6 | 17.7 | 142.9 | 70.9 |
| G347A | 104.3 | 42.1 | 95.2 | 1.0 | 127.4 | 8.2 | 132.6 | 38.4 |
| M348A | 92.3 | 30.9 | 101.1 | 11.4 | 116.2 | 14.7 | 125.7 | 2.1 |
| N349A | 84.9 | 33.7 | 91.0 | 2.6 | 112.7 | 12.2 | 141.0 | 27.5 |
| P350A | 93.8 | 12.4 | 107.7 | 18.5 | 93.2 | 12.1 | 173.0 | 21.7 |
| S351A | 88.9 | 8.0 | 112.5 | 3.3 | 116.9 | 7.4 | 153.5 | 27.9 |
| K352A | 112.5 | 14.7 | 94.1 | 18.2 | 115.1 | 3.2 | 155.5 | 33.1 |
| D353A | 74.6 | 12.7 | 92.1 | 4.2 | 109.3 | 16.4 | 149.3 | 7.6 |
| T354A | 84.7 | 4.9 | 99.3 | 3.0 | 112.8 | 6.5 | 150.2 | 5.9 |
| S355A | 126.0 | 19.2 | 98.9 | 8.3 | 116.7 | 4.5 | 157.9 | 17.1 |
| T356A | 89.3 | 33.9 | 99.5 | 1.0 | 90.2 | 25.5 | 180.9 | 7.8 |
| E357A | 75.3 | 12.3 | 91.2 | 7.5 | 109.7 | 6.6 | 148.3 | 11.0 |
| Y358A | 97.9 | 28.9 | 92.0 | 2.8 | 91.2 | 14.2 | 162.0 | 17.4 |
| S359A | 87.2 | 11.6 | 82.5 | 15.6 | 119.1 | 4.7 | 150.4 | 3.9 |
| E360A | 106.6 | 39.5 | 93.5 | 3.6 | 120.1 | 13.8 | 125.9 | 14.8 |
| V361A | 84.5 | 4.1 | 76.0 | 5.9 | 89.5 | 20.9 | 144.8 | 7.8 |
| R362A | 100.9 | 16.0 | 106.9 | 3.4 | 99.4 | 14.9 | 132.5 | 13.1 |
| T363A | 90.1 | 24.8 | 104.8 | 3.0 | 110.6 | 2.3 | 153.6 | 24.5 |
| Q364A | 106.6 | 15.9 | 94.4 | 10.3 | 108.9 | 4.3 | 130.4 | 14.0 |

### Example 5: Analysis and Comparison of the Identified Variants from the Alanine Scan

Table 9 summarizes the residues which, based on the data shown in Table 8, impact the binding of each of the tested antibodies to the indicated variants. Shown are all residues which led to a binding of the test antibodies of less than 40% as compared to the wild type CD33. Residues which led to a binding of less than 20% are shown in bold and are underlined.

TABLE 9

| Anti- | |
|---|---|
| body | Critical residues |
| Refmab | N20, F21, C41, H45, W60, C101, I105, Y116, F118, D205, |
| #1 | C212 |
| Refmab | C41, F43, F44, I47, W60, C101, Y116, F118, R122, D205, |
| #2 | C212 |
| Refmab | D18, N20, F21, C41, F44, P46, W60, C101, Y116, F118, M120, |
| #3 | Y127, Y129, D205 |
| Refmab | P19, L23, C41, F43, P48, Y49, Y50, D51, K52, W60, N76, |
| #4 | P96, C101, I105, D112, S115, Y116, F118, R122, F177, D205, |
| | C212 |
| Refmab | C41, Y49, K52, W60, C101, Y116, F118, D205, C212 |
| #5 | |
| Refmab | D18, W22, Q24, V25, C41, T42, F43, F44, W60, C101, I105, |
| #6 | Y116, F118, S131, P132, L134, D205, C212 |
| Refmab | C41, F44, Y49, Y50, D51, W60, C101, Y116, F118, R122, |
| #7 | D205, C212 |
| Refmab | C41, Y49, D51, K52, W60, C101, Y116, F118, D205 |
| #8 | |

As can be seen in Table 9, certain variants strongly block the binding of all antibodies. This is for example the case for variants C41, W60, C101, Y116, F118 and D205. The binding of all eight antibodies to these variants is decreased to below 40% as compared to wild type CD33. This is probably due to a major perturbance of the structure or loss of expression of these variants and not an indication that the changes amino acid positions are relevant for binding to the tested antibodies. These variants were therefore deselected.

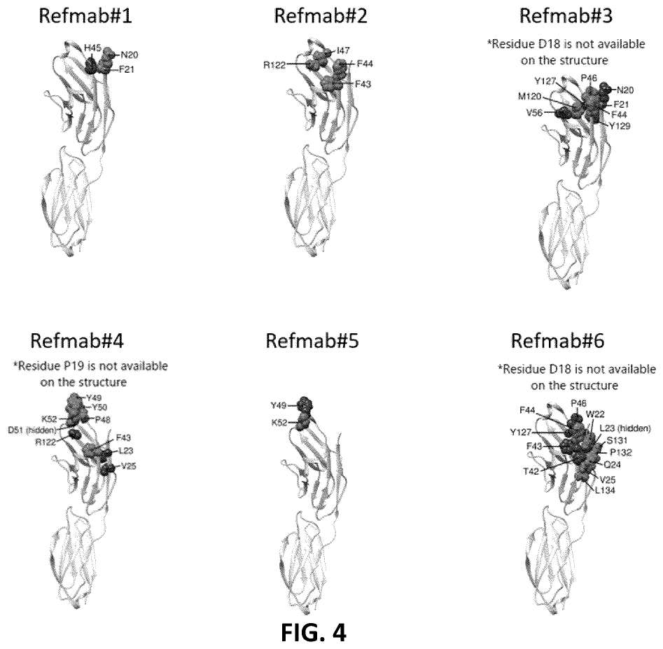

The identified potentially useful were plotted onto the 3D structure of CD33. See FIG. 4. This analysis, as well as additional criteria, such as the reproducibility of the binding activity, the surface accessibility, the structural localization and distance to other critical sites, as well as the nature and biochemical properties of the substituted amino acid (e.g. cysteine forming disulfide bridges or post-translational modification sites), were taken into account for selecting the residues for the next comprehensive mutational analysis.

### Example 6: Comprehensive Mutational Analysis

Based on the experimental data of Example 4 and the analysis of Example 5 certain amino acid residues were subjected to a more comprehensive mutational analysis, in which each residue was subject to a comprehensive mutagenesis to selected biophysically appropriate non-alanine amino acids, based on sequence and structure-related properties of the substituted amino acid as well as the newly introduced ones.

Antibodies were screened for binding to the human CD33 variants in IgG format. As in Example 4, binding of each test antibody to each mutant clone in the comprehensive library was determined in duplicate by high-throughput flow cytometry. For each mutation, background fluorescence was subtracted from the raw data, which were then normalized to antibody reactivity with wild-type CD33. Mean binding reactivities and ranges are listed in Table 10 for all mutant clones. Mutations that caused binding below 20% are highlighted in dark grey. Residues that led to binding of the test Abs between 20 and 40% as compared to the wild type CD33 are highlighted in light grey.

TABLE 10

| | Refmab #1 | | Refmab #2 | | Refmab #3 | | Refmab #4 | |
|---|---|---|---|---|---|---|---|---|
| | Mean % | Range % | Mean % | Range % | Mean % | Range % | Mean % | Range % |
| Muta- | WT | WT | WT | WT | WT | WT | WT | WT |
| tion | Binding | Binding | Binding | Binding | Binding | Binding | Binding | Binding |
| D18R | 60.6 | 6.5 | 96.5 | 2.3 | 5.3 | 0.0 | 100.3 | 2.2 |
| D18N | 69.6 | 0.2 | 59.2 | 5.6 | 71.5 | 6.6 | 76.0 | 5.3 |
| P19L | 91.6 | 3.0 | 111.4 | 12.2 | 117.3 | 0.1 | 100.7 | 3.8 |
| P19D | 2.6 | 1.2 | 99.3 | 5.1 | 0.6 | 0.4 | 94.8 | 1.4 |
| P19K | 5.5 | 3.9 | 115.2 | 0.3 | 1.8 | 2.6 | 92.0 | 0.1 |
| P19W | 73.8 | 2.3 | 88.3 | 7.0 | 71.2 | 3.9 | 82.8 | 0.9 |
| N20G | 5.1 | 3.8 | 105.4 | 8.7 | 3.6 | 0.1 | 103.3 | 24.3 |
| N20S | 45.5 | 9.0 | 123.8 | 23.0 | 4.6 | 2.1 | 91.7 | 7.8 |
| N20T | 6.9 | 0.3 | 116.0 | 27.3 | 2.2 | 0.6 | 92.1 | 10.8 |
| N20L | 78.5 | 0.8 | 95.8 | 5.8 | 9.4 | 0.8 | 89.8 | 4.7 |
| N20P | 2.9 | 1.1 | 98.5 | 5.6 | 1.7 | 0.5 | 98.5 | 3.5 |
| N20Y | 0.4 | 0.2 | 34.7 | 1.0 | 0.6 | 0.2 | 25.7 | 0.3 |
| N20W | 0.6 | 0.0 | 61.2 | | 0.7 | 0.6 | 52.1 | 0.7 |
| N20Q | 2.8 | 0.6 | 124.9 | 4.4 | 3.4 | 0.1 | 99.4 | 1.0 |
| N20D | 0.7 | 0.9 | 108.6 | 3.7 | 4.6 | 7.5 | 91.3 | 0.7 |
| N20E | 0.3 | 0.5 | 120.5 | 31.4 | 0.5 | 0.1 | 100.1 | 2.2 |
| N20K | 0.4 | 0.3 | 103.4 | 21.1 | 0.8 | 0.4 | 102.3 | 4.1 |
| N20V | 4.2 | 1.2 | 116.5 | 0.5 | 0.5 | 0.4 | 118.5 | 8.3 |
| F21S | 6.4 | 0.6 | 115.3 | 13.3 | 3.4 | 0.6 | 118.3 | 3.5 |
| F21V | 1.5 | 1.8 | 116.7 | 4.6 | 0.7 | 0.1 | 117.8 | 0.5 |
| F21L | 4.7 | 0.4 | 119.7 | 6.1 | 1.0 | 0.1 | 113.6 | 6.2 |
| F21I | 1.9 | 0.7 | 86.8 | 2.8 | 0.5 | 0.5 | 96.3 | 0.0 |
| F21H | 20.6 | 4.7 | 116.7 | 18.2 | 1.4 | 0.4 | 123.2 | 14.7 |
| W22G | 89.6 | 0.6 | 113.2 | 10.7 | 9.0 | 0.2 | 104.8 | 6.2 |
| W22S | 74.3 | 3.2 | 105.3 | 2.3 | 1.4 | 0.1 | 103.0 | 8.2 |
| W22T | 6.2 | 2.1 | 102.7 | 0.7 | 1.3 | 0.2 | 102.6 | 25.8 |
| W22L | 45.8 | 2.8 | 110.5 | 8.8 | 1.5 | 0.5 | 108.4 | 2.6 |
| W22P | 2.7 | 0.8 | 135.9 | 10.6 | 0.5 | 0.5 | 103.9 | 0.6 |
| W22Y | 98.0 | 3.9 | 107.1 | 4.1 | 11.5 | 1.6 | 86.1 | 4.1 |
| W22N | 76.5 | 0.9 | 106.7 | 18.3 | 3.5 | 0.6 | 100.2 | 5.4 |
| W22Q | 99.5 | 2.7 | 107.7 | 2.3 | 12.5 | 2.1 | 110.6 | 4.1 |
| W22E | 5.0 | 1.5 | 107.0 | 15.6 | 1.4 | 2.0 | 99.4 | 7.4 |
| W22R | 7.4 | 3.1 | 114.8 | 9.1 | 0.9 | 1.0 | 114.3 | 8.4 |
| L23M | 94.4 | 3.6 | 107.1 | 5.4 | 98.1 | 7.2 | 92.7 | 2.7 |
| Q24S | 60.2 | 3.6 | 108.2 | 1.9 | 5.3 | 0.9 | 96.8 | 1.6 |
| Q24T | 81.4 | 3.0 | 95.2 | 0.2 | 3.9 | 0.7 | 97.2 | 5.4 |
| Q24V | 92.6 | 3.9 | 99.3 | 0.7 | 24.5 | 1.6 | 81.5 | 4.0 |
| Q24I | 80.0 | 1.3 | 96.8 | 1.5 | 61.3 | 0.6 | 86.6 | 11.5 |
| Q24L | 80.9 | 0.4 | 102.2 | | 11.8 | 2.3 | 97.0 | 0.9 |
| Q24N | 86.5 | 1.9 | 117.2 | 2.7 | 35.3 | 1.5 | 106.8 | 8.4 |
| Q24H | 95.3 | 9.7 | 101.4 | 4.2 | 7.1 | 2.5 | 93.2 | 2.8 |
| Q24E | 17.1 | 0.1 | 63.7 | 1.0 | 0.8 | 1.2 | 69.7 | 2.5 |
| Q24R | 95.1 | 8.4 | 96.8 | 2.1 | 74.6 | 1.7 | 98.6 | 3.6 |
| V25S | 106.5 | 4.6 | 109.9 | 1.2 | 103.5 | 1.5 | 106.5 | 3.6 |
| V25I | 113.0 | 2.8 | 114.1 | 0.2 | 112.9 | 25.4 | 104.4 | 11.0 |
| V25L | 99.2 | 8.1 | 108.8 | 5.9 | 97.8 | 0.6 | 95.0 | 2.8 |
| V25M | 97.3 | 4.9 | 83.0 | 4.2 | 90.4 | 0.1 | 81.1 | 1.5 |
| T42N | 99.4 | 0.3 | 98.2 | 0.1 | 95.5 | 0.2 | 100.9 | 1.7 |
| T42K | 106.0 | 9.3 | 95.5 | 7.2 | 103.4 | 3.5 | 86.0 | 1.2 |
| T42P | 14.6 | 1.1 | 12.7 | 0.2 | 18.6 | 1.7 | 21.6 | 0.8 |
| F43V | 100.9 | 2.7 | 24.5 | 0.3 | 100.8 | 4.4 | 81.5 | 0.7 |
| F43L | 111.0 | 1.3 | 45.8 | 3.9 | 110.6 | 2.3 | 100.4 | 2.0 |
| F44S | 79.8 | 1.7 | 0.8 | 0.5 | 11.4 | 1.8 | 93.8 | 2.5 |
| F44V | 99.5 | 1.9 | 2.3 | 1.4 | 101.4 | 6.5 | 96.6 | 1.7 |
| F44I | 100.2 | 8.7 | 0.4 | 0.3 | 100.9 | 9.2 | 98.7 | 4.2 |
| F44L | 112.3 | 1.8 | 1.0 | 1.5 | 80.0 | 12.1 | 100.2 | 5.1 |
| F44P | 37.1 | 2.2 | 0.4 | 0.0 | 2.0 | 0.4 | 104.0 | 6.7 |
| F44Q | 106.0 | 2.3 | 0.5 | 0.4 | 105.0 | 2.0 | 102.1 | 6.8 |
| F44H | 97.6 | 3.7 | 0.4 | 0.0 | 91.2 | 3.1 | 84.9 | 4.4 |
| F44D | 98.9 | 10.2 | 2.0 | 3.2 | 1.2 | 0.2 | 109.6 | 3.8 |
| F44E | 92.7 | 2.4 | 1.7 | 0.2 | 33.3 | 2.2 | 112.5 | 2.1 |
| F44T | 106.7 | 5.4 | 0.8 | 0.0 | 83.3 | 8.6 | 97.1 | 8.3 |
| H45Y | 80.3 | 8.6 | 80.7 | 6.5 | 4.0 | 0.0 | 79.2 | 6.4 |
| P46S | 21.4 | 0.6 | 102.3 | 1.8 | 3.5 | 0.5 | 107.0 | 1.1 |
| P46I | 38.2 | 5.5 | 55.8 | 2.6 | 1.8 | 0.1 | 91.0 | 9.3 |
| P46L | 28.9 | 2.1 | 93.8 | 7.4 | 0.7 | 0.5 | 96.3 | 1.1 |
| P46F | 1.0 | 0.4 | 80.9 | 5.6 | 0.2 | 0.2 | 42.4 | 1.0 |
| P46R | 1.0 | 0.8 | 64.7 | 7.3 | 1.0 | 0.8 | 106.0 | |
| P46Y | 0.9 | 0.1 | 60.1 | 0.3 | 0.8 | 0.1 | 26.5 | 2.2 |
| P46H | 0.7 | 0.3 | 95.1 | 9.6 | 0.5 | 0.8 | 69.5 | 7.5 |
| P46D | 0.6 | 0.3 | 100.3 | 11.8 | 0.6 | 0.5 | 109.5 | 10.5 |
| I47G | 104.9 | 1.1 | 3.7 | 0.3 | 106.8 | 19.5 | 72.5 | 12.9 |
| I47S | 104.6 | 0.4 | 3.4 | 1.0 | 94.9 | 3.2 | 90.1 | 6.9 |
| I47T | 97.2 | 3.3 | 1.8 | 0.3 | 72.4 | 7.3 | 82.4 | 4.7 |
| I47V | 112.1 | 15.6 | 97.2 | 6.2 | 100.1 | 4.3 | 99.0 | 1.0 |
| I47L | 112.3 | 5.2 | 2.8 | 0.2 | 102.2 | 2.3 | 88.5 | 0.1 |
| I47F | 101.0 | 2.2 | 2.2 | 0.5 | 101.6 | 4.7 | 102.4 | 0.7 |
| I47Y | 110.1 | 1.7 | 1.4 | 0.8 | 108.6 | 10.8 | 96.0 | 16.2 |
| I47W | 111.4 | 10.5 | 0.6 | 0.6 | 112.5 | 10.4 | 86.8 | 4.2 |
| I47Q | 104.5 | 4.1 | 1.0 | 1.5 | 105.9 | 12.4 | 80.1 | 2.7 |
| I47E | 101.1 | 0.2 | 0.5 | 0.8 | 109.4 | 8.1 | 54.8 | 2.5 |
| I47R | 96.1 | 0.9 | 1.6 | 0.7 | 83.2 | 1.2 | 66.2 | 6.1 |
| I47P | 84.5 | 9.5 | 0.7 | 0.4 | 2.8 | 0.7 | 49.0 | 5.9 |
| I47N | 87.8 | 3.7 | 0.4 | 0.6 | 91.6 | 3.5 | 74.8 | 2.4 |
| P48T | 106.5 | 2.6 | 71.3 | 9.9 | 102.3 | 3.7 | 11.7 | 1.2 |
| P48H | 97.4 | 0.6 | 81.9 | 4.1 | 87.6 | 6.8 | 2.9 | 4.5 |
| P48V | 120.9 | 7.0 | 79.5 | 5.1 | 113.1 | 15.3 | 88.5 | 3.7 |
| P48I | 106.6 | 3.0 | 81.9 | 8.1 | 122.3 | 17.8 | 31.9 | 6.4 |
| P48L | 101.6 | 3.2 | 90.4 | 1.1 | 110.8 | 4.6 | 1.0 | 0.9 |
| P48F | 94.6 | 9.8 | 68.7 | 6.3 | 100.5 | 13.3 | 0.3 | 0.1 |
| P48Y | 99.5 | 3.3 | 21.6 | 0.1 | 90.3 | 12.4 | 0.6 | 0.3 |
| P48M | 110.3 | 7.0 | 87.5 | 1.9 | 106.4 | 4.2 | 1.3 | 0.7 |
| P48W | 95.6 | 3.0 | 68.8 | 4.6 | 96.4 | 1.1 | 1.3 | 0.2 |
| P48N | 100.1 | 4.2 | 95.2 | | 98.8 | 11.5 | 3.8 | 1.7 |
| P48Q | 120.1 | 6.3 | 105.6 | 1.7 | 100.9 | 12.9 | 2.1 | 2.2 |
| P48D | 108.8 | 0.2 | 10.4 | 3.3 | 100.2 | 12.2 | 2.0 | 0.1 |
| P48K | 99.4 | 3.5 | 106.4 | 4.2 | 67.3 | 6.5 | 0.0 | 0.0 |
| P48G | 95.0 | 9.6 | 80.8 | 9.5 | 108.3 | 3.3 | 0.4 | 0.3 |
| P48S | 97.5 | 3.1 | 92.1 | 5.7 | 100.5 | 2.8 | 0.5 | 0.0 |
| Y49P | 92.5 | 18.3 | 6.9 | 8.5 | 92.1 | 2.3 | 1.2 | 1.4 |
| Y49Q | 104.6 | 9.9 | 17.4 | 0.3 | 101.9 | 4.8 | 91.9 | 8.9 |
| Y49H | 89.1 | 2.2 | 17.2 | 6.2 | 92.1 | 5.9 | 95.1 | 3.5 |
| Y49D | 93.4 | 3.2 | 1.4 | 0.8 | 94.4 | 3.6 | 2.5 | 0.9 |
| Y49R | 87.1 | 0.2 | 44.9 | | 79.0 | 2.1 | 55.7 | 2.9 |
| Y49V | 100.9 | 0.3 | 90.4 | 5.6 | 105.8 | 19.0 | 46.9 | 0.0 |
| Y49G | 107.3 | 1.1 | 6.8 | 7.0 | 107.9 | 17.8 | 54.9 | 4.3 |
| Y49C | 67.2 | 8.5 | 3.2 | 0.5 | 59.2 | 0.2 | 0.5 | 0.3 |
| Y49S | 107.1 | 2.7 | 87.2 | 4.4 | 106.7 | 3.6 | 9.8 | 0.1 |
| Y49T | 108.0 | 0.1 | 76.3 | 7.6 | 96.5 | 11.6 | 2.8 | 0.5 |
| Y49I | 109.2 | 1.8 | 94.3 | 2.9 | 109.3 | 19.9 | 94.0 | 1.7 |
| Y49L | 100.5 | 10.1 | 107.6 | 1.6 | 93.8 | 11.7 | 110.3 | 1.7 |
| Y50S | 101.2 | 5.6 | 122.7 | 3.4 | 97.2 | 3.9 | 2.9 | 1.3 |
| Y50V | 94.9 | 9.4 | 125.9 | 2.4 | 108.0 | 4.0 | 1.7 | 0.2 |
| Y50I | 99.2 | 12.4 | 125.8 | 2.8 | 92.8 | 4.7 | 4.5 | 0.6 |
| Y50N | 96.3 | 1.7 | 107.6 | 3.6 | 92.4 | 3.9 | 2.3 | 1.7 |
| Y50Q | 93.9 | 1.6 | 112.9 | 15.8 | 95.4 | 1.9 | 3.2 | 5.8 |
| Y50H | 103.2 | 11.2 | 119.3 | 9.3 | 110.8 | 2.4 | 2.0 | 0.3 |
| Y50D | 114.7 | 6.2 | 112.5 | 9.9 | 100.2 | 4.6 | 0.9 | 1.3 |
| Y50R | 94.1 | 8.6 | 109.3 | 0.4 | 83.0 | 0.7 | 0.3 | 0.3 |
| Y50G | 92.8 | 13.5 | 104.5 | 23.1 | 91.2 | 2.2 | 4.0 | 2.1 |
| Y50L | 109.1 | 13.4 | 119.3 | 11.9 | 105.5 | 5.2 | 1.9 | 0.8 |
| Y50P | 98.1 | 7.0 | 95.8 | 2.9 | 102.8 | 8.7 | 85.6 | 1.5 |
| D51K | 90.7 | 1.0 | 2.7 | 0.9 | 88.9 | 2.6 | 42.0 | 2.4 |
| D51Y | 94.4 | 23.8 | 69.8 | 9.3 | 87.2 | 2.5 | 61.9 | 1.5 |
| D51N | 99.0 | 14.8 | 96.7 | 2.8 | 106.6 | 14.3 | 76.4 | 4.7 |
| K52G | 98.8 | 3.2 | 25.4 | 1.7 | 106.0 | 3.4 | 69.5 | 1.3 |
| K52S | 84.3 | 4.6 | 113.3 | 13.4 | 96.8 | 3.0 | 85.0 | 3.6 |
| K52L | 97.6 | 4.6 | 74.8 | 6.7 | 94.9 | 5.4 | 88.1 | 4.1 |
| K52F | 4.5 | 0.8 | 3.9 | 4.1 | 6.0 | 3.5 | 3.5 | 1.8 |
| K52Y | 45.0 | 1.2 | 21.0 | 1.3 | 41.7 | 0.6 | 20.7 | 2.1 |
| K52D | 101.2 | 2.1 | 5.3 | 1.2 | 101.1 | 1.8 | 9.4 | 1.0 |
| K52E | 98.6 | 9.4 | 52.4 | 0.6 | 105.5 | 5.8 | 51.4 | 4.0 |
| K52H | 94.2 | 5.9 | 84.7 | 11.2 | 101.3 | 5.7 | 75.1 | 2.8 |
| K52T | 101.7 | 10.4 | 119.6 | 1.7 | 102.7 | 2.9 | 83.5 | 8.0 |
| K52I | 89.2 | 5.6 | 86.8 | 5.0 | 88.7 | 4.1 | 61.9 | 1.5 |
| V56R | 98.5 | 0.1 | 107.2 | 2.8 | 97.7 | 9.6 | 99.4 | |
| M120V | 98.7 | 2.8 | 95.5 | 1.7 | 51.1 | 3.1 | 100.4 | 3.7 |
| M120I | 96.6 | 12.7 | 95.7 | 1.1 | 81.6 | 1.1 | 100.8 | 6.0 |
| M120L | 98.4 | 4.8 | 123.6 | 2.0 | 90.8 | 4.1 | 109.9 | 13.6 |
| R122G | 109.0 | 27.2 | 12.2 | 2.5 | 114.3 | 15.2 | 91.3 | 6.7 |
| R122S | 110.8 | 8.4 | 3.4 | 2.2 | 111.0 | 2.0 | 90.2 | 0.5 |
| R122I | 109.5 | 0.3 | 1.1 | 0.0 | 112.4 | 12.2 | 85.8 | 0.8 |
| R122F | 110.5 | 7.6 | 1.2 | 1.7 | 105.6 | 2.1 | 58.1 | 1.5 |
| R122E | 110.1 | 15.3 | 0.6 | 0.6 | 100.9 | 0.1 | 79.2 | 0.3 |
| R122T | 113.1 | 9.5 | 1.5 | 0.0 | 103.0 | 6.3 | 93.2 | 6.0 |
| Y127H | 97.1 | 10.0 | 113.6 | 5.9 | 75.4 | 4.2 | 105.3 | 3.2 |
| Y127E | 79.2 | 3.6 | 104.8 | 7.8 | 21.5 | 0.5 | 104.2 | 12.2 |
| Y127R | 84.0 | 17.6 | 110.5 | 14.4 | 65.9 | 9.0 | 115.5 | 9.4 |
| Y129S | 68.7 | 8.0 | 101.9 | 10.5 | 7.6 | 7.4 | 107.1 | 5.7 |
| S131G | 95.1 | 4.6 | 112.3 | 3.6 | 100.0 | 2.5 | 96.9 | 2.1 |
| S131T | 97.2 | 4.8 | 111.3 | 2.2 | 116.3 | 0.2 | 102.3 | 0.3 |
| S131N | 94.6 | 11.0 | 125.6 | 7.0 | 118.3 | 23.1 | 105.4 | 0.2 |
| S131H | 96.4 | 9.1 | 112.0 | 3.3 | 100.2 | 12.6 | 102.6 | 0.7 |
| S131D | 91.9 | 5.3 | 101.4 | 5.6 | 93.9 | 7.4 | 97.5 | 5.1 |
| S131K | 101.7 | 8.9 | 91.2 | 1.1 | 87.7 | 3.8 | 100.3 | 7.5 |
| S131I | 96.6 | 9.9 | 109.7 | 4.3 | 95.7 | 5.5 | 99.6 | 0.6 |
| S131F | 95.6 | 5.3 | 110.6 | 0.4 | 102.9 | 4.3 | 91.8 | 2.9 |
| S131Y | 97.7 | 17.2 | 98.5 | 5.8 | 97.4 | 11.9 | 85.7 | 3.9 |
| S131M | 105.0 | 1.7 | 108.1 | 4.8 | 91.9 | 5.2 | 97.6 | 4.1 |
| S131W | 97.6 | 7.8 | 92.4 | 15.0 | 91.9 | 3.4 | 87.5 | 1.4 |
| S131Q | 101.3 | 5.7 | 109.7 | 13.1 | 93.9 | 6.2 | 97.3 | 2.2 |
| P132S | 92.9 | 6.0 | 109.7 | 2.4 | 95.8 | 1.7 | 102.4 | 0.8 |
| P132T | 98.9 | 6.6 | 103.1 | 5.2 | 105.8 | 18.0 | 99.8 | 3.1 |
| P132V | 105.4 | 2.7 | 102.4 | 4.1 | 86.6 | 2.2 | 94.7 | 0.6 |
| P132L | 94.6 | 12.3 | 115.8 | 5.4 | 94.4 | 13.2 | 96.7 | 6.6 |
| P132F | 91.3 | 1.8 | 65.1 | 5.5 | 79.4 | 2.0 | 71.3 | 5.0 |
| P132Y | 93.5 | 9.3 | 65.0 | 1.4 | 85.2 | 2.9 | 78.6 | 1.4 |
| P132H | 84.9 | 9.6 | 100.7 | 6.7 | 87.8 | 2.0 | 92.7 | 0.3 |
| P132D | 89.9 | 3.1 | 73.6 | 2.8 | 92.7 | 1.6 | 99.6 | 0.8 |
| P132K | 99.6 | 19.5 | 118.7 | 9.0 | 65.0 | 3.5 | 101.8 | 1.0 |
| P132N | 91.4 | 4.9 | 100.2 | 13.4 | 94.5 | 5.2 | 100.3 | 0.4 |
| P132Q | 91.7 | 1.1 | 83.4 | 9.1 | 80.1 | 8.7 | 86.9 | 8.4 |
| L134T | 91.6 | 6.7 | 99.2 | 13.6 | 101.3 | 6.2 | 90.3 | 4.1 |
| L134I | 106.3 | 9.3 | 105.3 | 5.2 | 108.3 | 16.5 | 94.4 | 0.9 |
| L134Q | 83.0 | 1.4 | 77.4 | 3.5 | 87.7 | 0.6 | 86.8 | 1.1 |
| L134R | 3.1 | 2.0 | 2.5 | | 1.0 | 0.5 | 2.4 | 0.4 |
| L134F | 92.5 | 9.8 | 105.9 | 13.2 | 104.4 | 4.3 | 90.6 | 0.4 |
| L134M | 81.2 | 23.0 | 104.6 | 8.8 | 105.6 | 0.2 | 99.0 | 2.8 |
| | Refmab #5 | | Refmab #6 | | Refmab #7 | | Refmab #8 | |
| | Mean % | Range % | Mean % | Range % | Mean % | Range % | Mean % | Range % |
| Muta- | WT | WT | WT | WT | WT | WT | WT | WT |
| tion | Binding | Binding | Binding | Binding | Binding | Binding | Binding | Binding |
| D18R | 97.4 | 7.8 | 68.3 | 3.7 | 101.9 | 4.9 | 97.1 | 2.0 |
| D18N | 58.7 | 2.8 | 32.2 | 3.0 | 40.9 | 2.6 | 54.2 | 14.7 |
| P19L | 100.4 | 4.4 | 103.2 | 10.7 | 113.5 | 1.3 | 99.9 | 17.3 |
| P19D | 96.4 | 4.5 | 105.4 | 11.2 | 122.1 | 2.4 | 113.4 | 15.4 |
| P19K | 86.2 | 3.6 | 85.4 | 2.8 | 97.3 | 7.9 | 97.2 | 13.0 |
| P19W | 85.9 | 7.2 | 75.5 | 11.8 | 84.1 | 28.9 | 102.9 | 0.9 |
| N20G | 96.9 | 9.4 | 101.2 | 7.4 | 81.5 | 2.1 | 112.3 | 5.1 |
| N20S | 95.1 | 13.1 | 101.2 | 7.8 | 109.1 | 12.4 | 105.6 | 2.5 |
| N20T | 107.3 | 16.1 | 106.5 | 1.0 | 105.7 | 3.1 | 112.6 | 1.2 |
| N20L | 96.3 | 17.2 | 88.2 | 0.6 | 84.1 | 2.1 | 104.0 | 2.9 |
| N20P | 96.8 | 1.0 | 97.2 | 0.7 | 127.2 | 4.9 | 105.1 | 3.2 |
| N20Y | 57.7 | 4.0 | 28.0 | 0.3 | 22.6 | 2.0 | 55.1 | 5.5 |
| N20W | 71.7 | 1.4 | 50.3 | 2.4 | 45.4 | 0.4 | 78.9 | 1.8 |
| N20Q | 93.2 | 5.7 | 97.2 | 4.1 | 102.5 | 1.3 | 92.7 | 5.4 |
| N20D | 97.1 | 3.4 | 102.7 | 9.0 | 70.6 | 3.7 | 105.5 | 22.5 |
| N20E | 108.9 | 15.1 | 108.5 | 5.5 | 79.9 | 0.9 | 114.4 | 7.2 |
| N20K | 105.4 | 13.7 | 91.8 | 12.3 | 114.9 | 11.6 | 96.6 | 1.3 |
| N20V | 108.5 | 12.3 | 96.9 | 11.4 | 112.3 | 2.1 | 106.6 | 4.1 |
| F21S | 111.6 | 18.0 | 115.0 | 23.2 | 175.8 | 39.7 | 121.9 | 2.5 |
| F21V | 104.1 | 4.2 | 118.5 | 3.2 | 141.2 | 9.4 | 117.1 | 2.0 |
| F21L | 122.3 | 1.2 | 117.7 | 1.1 | 140.1 | 12.2 | 113.2 | 5.4 |
| F21I | 95.7 | 5.9 | 89.3 | 6.8 | 90.2 | 4.7 | 95.2 | 14.4 |
| F21H | 107.9 | 8.2 | 111.4 | 6.3 | 152.5 | 23.9 | 110.2 | 22.9 |
| W22G | 100.6 | 2.9 | 8.1 | 1.3 | 92.0 | 0.7 | 106.4 | 3.3 |
| W22S | 89.7 | 3.0 | 6.7 | 0.3 | 74.2 | 2.4 | 99.1 | 1.6 |
| W22T | 107.9 | 5.0 | 2.6 | 0.3 | 82.4 | 13.8 | 101.9 | 3.2 |
| W22L | 96.0 | 8.9 | 1.1 | 0.5 | 81.3 | 0.0 | 95.7 | |
| W22P | 110.5 | 11.5 | 1.8 | 0.0 | 125.9 | 1.5 | 119.3 | 3.5 |
| W22Y | 107.3 | 5.1 | 4.1 | 0.3 | 96.0 | 0.3 | 99.8 | 4.3 |
| W22N | 106.3 | 8.3 | 5.6 | 0.3 | 94.8 | 6.6 | 93.3 | 12.1 |
| W22Q | 109.0 | 5.4 | 7.6 | 1.2 | 80.8 | 0.1 | 93.1 | |
| W22E | 111.1 | 12.2 | 1.7 | 0.2 | 80.5 | 15.7 | 100.2 | 25.1 |
| W22R | 104.3 | 1.7 | 1.3 | 0.2 | 90.2 | 27.2 | 65.9 | 16.1 |
| L23M | 90.7 | 11.8 | 100.4 | 5.5 | 80.7 | 0.7 | 112.3 | 3.5 |
| Q24S | 110.5 | 12.6 | 80.7 | 13.7 | 93.9 | 4.4 | 118.2 | 2.2 |
| Q24T | 105.4 | 3.2 | 97.7 | 6.6 | 90.5 | 6.8 | 108.5 | 8.1 |
| Q24V | 97.9 | 2.0 | 94.3 | 0.8 | 84.2 | 12.1 | 101.3 | 3.8 |
| Q24I | 94.6 | 3.8 | 89.9 | 3.4 | 74.3 | 2.1 | 110.2 | 2.7 |
| Q24L | 101.0 | 3.1 | 98.9 | 1.9 | 83.9 | 9.8 | 113.2 | 5.5 |
| Q24N | 109.7 | 2.1 | 95.4 | 0.1 | 106.8 | 9.7 | 104.7 | 7.6 |
| Q24H | 104.0 | 6.5 | 100.1 | 3.0 | 107.7 | 34.0 | 102.6 | 14.6 |
| Q24E | 95.6 | 7.5 | 89.7 | 0.2 | 65.1 | 4.3 | 90.8 | 1.1 |
| Q24R | 102.4 | 7.5 | 86.2 | 7.4 | 124.0 | 40.1 | 93.5 | 2.5 |
| V25S | 107.3 | 7.6 | 94.7 | 2.6 | 102.7 | 12.1 | 100.8 | 3.6 |
| V25I | 98.9 | 0.3 | 106.1 | 3.7 | 95.1 | 8.2 | 113.7 | |
| V25L | 94.9 | 8.4 | 104.5 | 7.5 | 103.0 | 9.8 | 121.3 | 8.1 |
| V25M | 87.6 | 1.3 | 68.6 | 6.5 | 65.1 | 9.4 | 90.5 | 6.6 |
| T42N | 91.1 | 7.7 | 93.5 | 2.5 | 95.4 | 14.2 | 97.6 | 6.2 |
| T42K | 79.5 | 4.1 | 91.2 | 9.1 | 77.0 | 18.7 | 91.7 | 9.8 |
| T42P | 13.9 | 1.6 | 9.3 | 1.0 | 15.9 | 4.4 | 13.4 | 1.7 |
| F43V | 82.2 | 3.2 | 91.8 | 0.2 | 73.4 | 6.3 | 79.1 | 5.9 |
| F43L | 96.3 | 1.5 | 87.9 | 5.8 | 94.0 | 14.7 | 105.6 | 6.5 |
| F44S | 103.8 | 2.2 | 86.1 | 5.1 | 31.8 | 3.8 | 46.7 | 7.5 |
| F44V | 97.7 | 13.2 | 98.9 | 3.6 | 36.1 | 0.6 | 80.8 | 6.0 |
| F44I | 105.5 | 0.6 | 93.1 | 2.6 | 46.1 | 5.0 | 78.3 | 11.5 |
| F44L | 109.1 | 16.0 | 103.7 | 2.8 | 99.6 | 5.2 | 49.3 | 2.1 |
| F44P | 113.5 | 4.5 | 88.0 | 4.8 | 21.8 | 4.0 | 4.1 | 0.3 |
| F44Q | 93.9 | 1.2 | 101.7 | 2.9 | 74.8 | 4.9 | 28.9 | 2.3 |
| F44H | 116.6 | 20.8 | 91.3 | 5.1 | 82.4 | 1.2 | 70.0 | 9.3 |
| F44D | 111.4 | 3.9 | 106.7 | 5.6 | 9.5 | 0.5 | 8.6 | 2.7 |
| F44E | 97.0 | 16.8 | 102.3 | 5.3 | 18.3 | 3.0 | 16.4 | 0.8 |
| F44T | 92.4 | 0.7 | 93.9 | 1.6 | 31.7 | 13.6 | 51.6 | 1.1 |
| H45Y | 94.2 | 3.2 | 84.4 | 4.8 | 75.7 | 0.1 | 113.0 | 26.9 |
| P46S | 98.7 | 10.2 | 109.9 | 4.4 | 96.4 | 7.8 | 112.8 | 8.4 |
| P46I | 100.3 | 3.4 | 98.5 | 4.5 | 106.7 | 2.9 | 90.2 | 16.1 |
| P46L | 87.2 | 15.2 | 107.0 | 2.6 | 66.8 | 1.4 | 108.0 | 1.5 |
| P46F | 62.0 | 9.9 | 92.6 | 1.0 | 23.0 | 2.2 | 87.9 | 2.2 |
| P46R | 102.7 | 6.4 | 102.8 | 6.9 | 33.4 | 6.6 | 96.0 | |
| P46Y | 48.0 | 1.4 | 79.9 | 5.4 | 8.6 | 1.8 | 78.8 | 8.1 |
| P46H | 69.8 | 0.7 | 107.3 | 1.2 | 94.3 | 11.3 | 80.9 | 2.2 |
| P46D | 76.0 | 3.0 | 104.0 | 21.8 | 15.9 | 0.6 | 82.3 | 2.5 |
| I47G | 80.9 | 9.8 | 104.7 | 33.6 | 15.1 | 1.5 | 0.3 | 0.1 |
| I47S | 98.2 | 4.6 | 91.0 | 15.7 | 33.6 | 7.2 | 63.5 | 7.1 |
| I47T | 98.4 | 3.9 | 97.2 | 2.6 | 32.7 | 6.1 | 79.6 | 8.1 |
| I47V | 104.2 | 2.9 | 100.7 | 12.7 | 97.4 | 4.9 | 102.5 | 9.8 |
| I47L | 106.7 | 2.9 | 99.1 | 1.2 | 37.4 | 4.5 | 2.0 | 0.9 |
| I47F | 90.5 | 1.1 | 104.8 | 3.1 | 85.2 | 9.3 | 75.7 | 8.1 |
| I47Y | 94.5 | 2.6 | 104.8 | 2.3 | 79.6 | 15.2 | 81.2 | 12.2 |
| I47W | 90.8 | 0.3 | 114.1 | 2.5 | 45.4 | 0.2 | 99.4 | 17.3 |
| I47Q | 94.6 | 4.3 | 107.4 | 1.5 | 8.2 | 0.1 | 1.6 | 0.0 |
| I47E | 89.1 | 6.1 | 121.6 | 1.9 | 3.9 | 0.2 | 0.2 | 0.7 |
| I47R | 94.6 | 0.5 | 96.9 | 1.5 | 2.0 | 0.9 | 0.8 | 0.3 |
| I47P | 4.9 | 0.5 | 113.8 | 12.1 | 1.2 | 0.5 | 1.0 | 0.9 |
| I47N | 88.0 | 13.5 | 96.6 | 3.2 | 4.7 | 1.5 | 0.8 | 0.7 |
| P48T | 91.4 | 16.5 | 92.6 | 6.5 | 1.6 | 0.4 | 5.8 | 1.7 |
| P48H | 7.4 | 2.8 | 94.4 | 1.0 | 1.6 | 2.8 | 76.0 | 0.8 |
| P48V | 53.5 | 13.0 | 105.1 | 7.5 | 0.9 | 0.0 | 83.9 | 23.1 |
| P48I | 4.0 | 0.1 | 102.1 | 0.7 | 0.1 | 0.0 | 66.2 | 12.2 |
| P48L | 18.9 | 0.6 | 103.6 | 6.5 | 0.3 | 0.4 | 59.0 | 9.6 |
| P48F | 6.0 | 1.0 | 93.8 | 4.4 | 0.2 | 0.0 | 62.7 | 10.8 |
| P48Y | 1.5 | 0.0 | 79.1 | 4.3 | 0.5 | 1.0 | 24.5 | 4.3 |
| P48M | 31.4 | 0.8 | 103.1 | 0.6 | 3.2 | 0.6 | 87.7 | 0.0 |
| P48W | 2.2 | 2.1 | 87.2 | 1.4 | 0.7 | 0.6 | 88.9 | |
| P48N | 4.4 | 0.4 | 98.4 | 7.8 | 4.3 | 0.5 | 19.9 | 4.1 |
| P48Q | 2.1 | 0.7 | 111.1 | 0.4 | 1.3 | 0.2 | 64.6 | 5.8 |
| P48D | 2.1 | 0.1 | 115.0 | 6.2 | 0.1 | 0.2 | 0.1 | 0.1 |
| P48K | 58.7 | 1.5 | 89.4 | 3.6 | 1.6 | 1.4 | 87.6 | 0.4 |
| P48G | 3.5 | 1.3 | 89.0 | 9.1 | 13.3 | 3.6 | 21.4 | 15.2 |
| P48S | 43.0 | 10.5 | 97.2 | 2.7 | 2.6 | 2.1 | 7.7 | 0.7 |
| Y49P | 2.2 | 1.9 | 116.6 | 11.7 | 11.7 | 2.6 | 1.9 | 3.3 |
| Y49Q | 2.3 | 1.1 | 114.8 | 2.2 | 0.7 | 0.9 | 0.2 | 0.4 |
| Y49H | 2.4 | 2.7 | 118.1 | 2.0 | 27.8 | 3.3 | 19.6 | 0.7 |
| Y49D | 1.7 | 2.0 | 117.4 | 10.0 | 3.9 | 0.8 | 0.4 | 1.4 |
| Y49R | 1.0 | 1.1 | 101.4 | 3.2 | 1.6 | 0.4 | 0.4 | 0.7 |
| Y49V | 0.9 | 0.3 | 121.1 | 3.9 | 0.9 | 0.5 | 0.3 | 0.5 |
| Y49G | 0.8 | 0.8 | 121.4 | 1.0 | 20.5 | 3.6 | 4.0 | |
| Y49C | 0.7 | 0.6 | 44.7 | 4.0 | 0.6 | 0.0 | 0.1 | 0.4 |
| Y49S | 0.5 | 0.0 | 102.0 | 0.6 | 3.6 | 1.5 | 1.2 | 1.6 |
| Y49T | 0.5 | 0.5 | 120.7 | 1.2 | 0.1 | 0.3 | 0.7 | |
| Y49I | 0.5 | 0.4 | 112.9 | 3.6 | 0.2 | 0.4 | 0.3 | 0.5 |
| Y49L | 0.8 | 1.0 | 97.9 | 8.0 | 1.6 | 0.2 | 0.0 | 0.3 |
| Y50S | 5.3 | 1.6 | 122.0 | 5.9 | 7.7 | 3.3 | 66.8 | 7.5 |
| Y50V | 4.2 | 3.8 | 108.3 | 3.9 | 3.9 | 0.9 | 64.6 | 1.5 |
| Y50I | 2.5 | 1.1 | 118.1 | 3.7 | 5.1 | 1.4 | 69.2 | 1.8 |
| Y50N | 3.2 | 1.5 | 107.6 | 10.1 | 4.7 | 2.6 | 78.1 | 4.5 |
| Y50Q | 2.9 | 3.5 | 103.3 | 4.3 | 7.2 | 3.4 | 74.5 | 1.7 |
| Y50H | 7.1 | 2.7 | 92.2 | 35.5 | 18.0 | 0.1 | 101.9 | 4.4 |
| Y50D | 2.5 | 2.9 | 129.2 | 7.8 | 1.2 | 2.4 | 76.2 | 6.2 |
| Y50R | 47.0 | 6.7 | 96.9 | 1.5 | 1.7 | 2.2 | 43.1 | 3.6 |
| Y50G | 5.0 | 3.6 | 115.1 | 11.2 | 17.2 | 25.3 | 70.0 | 9.9 |
| Y50L | 1.7 | 1.0 | 107.9 | 5.2 | 4.7 | 3.4 | 73.2 | 8.4 |
| Y50P | 43.4 | 0.8 | 98.1 | 5.5 | 0.4 | 0.3 | 36.0 | 0.3 |
| D51K | 4.2 | 1.8 | 77.7 | 0.3 | 1.5 | 2.3 | 1.4 | 1.7 |
| D51Y | 57.1 | 3.8 | 71.3 | 4.5 | 2.0 | 0.7 | 0.9 | 0.9 |
| D51N | 89.4 | 20.1 | 92.6 | 1.8 | 64.4 | 9.5 | 97.2 | 0.7 |
| K52G | 2.6 | 2.2 | 104.6 | 4.1 | 12.9 | 1.6 | 0.3 | 0.1 |
| K52S | 3.8 | 3.9 | 107.2 | 6.5 | 28.8 | 3.7 | 1.1 | 1.0 |
| K52L | 2.2 | 1.5 | 99.4 | 3.8 | 88.7 | 2.8 | 1.4 | 2.2 |
| K52F | 2.5 | 0.7 | 2.5 | 0.9 | 2.4 | 1.2 | 1.1 | 1.9 |
| K52Y | 2.0 | 1.7 | 26.3 | 1.7 | 18.7 | 2.8 | 0.7 | 1.1 |
| K52D | 3.2 | 3.2 | 125.8 | 5.3 | 3.6 | 1.0 | 2.0 | 0.9 |
| K52E | 13.8 | 1.7 | 121.2 | 2.4 | 14.4 | 0.6 | 7.6 | 1.4 |
| K52H | 59.7 | 1.3 | 86.1 | 0.2 | 52.1 | 4.0 | 15.3 | 0.9 |
| K52T | 5.0 | 1.4 | 105.9 | 12.0 | 53.1 | 5.0 | 0.9 | 1.1 |
| K52I | 1.9 | 0.1 | 71.6 | 6.3 | 56.2 | 9.2 | 0.5 | 0.3 |
| V56R | 87.4 | 4.8 | 93.2 | 7.0 | 113.6 | 8.0 | 113.2 | 3.0 |
| M120V | 85.9 | 6.3 | 97.0 | 16.5 | 82.9 | 11.2 | 108.5 | 27.5 |
| M120I | 93.0 | 4.0 | 101.5 | 3.8 | 105.7 | 4.8 | 143.3 | 42.0 |
| M120L | 103.1 | 4.6 | 101.1 | 9.4 | 106.7 | 6.6 | 123.5 | 1.3 |
| R122G | 94.9 | 0.8 | 111.7 | 4.8 | 13.6 | 0.9 | 28.5 | 2.1 |
| R122S | 90.6 | 11.7 | 108.5 | 0.2 | 4.9 | 2.5 | 33.8 | 0.1 |
| R122I | 86.1 | 5.1 | 107.4 | 5.7 | 5.6 | 1.6 | 8.1 | 2.9 |
| R122F | 90.1 | 11.4 | 92.5 | 6.7 | 1.2 | 0.6 | 1.1 | 1.8 |
| R122E | 95.8 | 5.4 | 96.0 | 34.1 | 6.2 | 0.3 | 67.8 | 0.4 |
| R122T | 83.2 | 11.1 | 113.6 | 2.7 | 13.9 | 0.9 | 88.3 | 2.4 |
| Y127H | 97.2 | 4.1 | 111.7 | 7.1 | 129.0 | 17.7 | 101.7 | 5.8 |
| Y127E | 93.9 | 4.5 | 101.8 | 2.0 | 104.3 | 4.9 | 104.6 | 30.5 |
| Y127R | 108.2 | 5.1 | 107.7 | 0.1 | 129.2 | 6.1 | 92.3 | 22.8 |
| Y129S | 94.7 | 11.6 | 99.6 | 2.5 | 113.5 | 5.6 | 120.8 | 46.0 |
| S131G | 97.1 | 11.6 | 101.0 | 8.9 | 86.4 | 4.3 | 105.7 | 6.1 |
| S131T | 89.0 | 1.4 | 99.5 | 1.9 | 102.2 | 14.5 | 107.6 | 3.8 |
| S131N | 101.1 | 5.2 | 84.6 | 5.6 | 104.8 | 5.1 | 113.6 | 4.0 |
| S131H | 96.8 | 3.1 | 68.5 | 4.2 | 113.2 | 14.3 | 111.0 | 4.7 |
| S131D | 101.8 | 1.3 | 88.9 | 6.8 | 95.6 | 8.4 | 97.1 | 7.6 |
| S131K | 96.4 | 3.5 | 2.5 | 1.2 | 116.0 | 0.2 | 96.0 | 6.3 |
| S131I | 84.8 | 7.2 | 24.0 | 5.1 | 93.5 | 9.2 | 90.5 | 2.0 |
| S131F | 82.3 | 4.8 | 2.4 | 1.5 | 75.2 | 17.1 | 104.3 | 1.7 |
| S131Y | 89.5 | 2.5 | 24.5 | 4.5 | 72.4 | 10.2 | 97.6 | 0.5 |
| S131M | 83.8 | 1.6 | 4.3 | 1.9 | 96.3 | 6.7 | 96.7 | 6.5 |
| S131W | 94.5 | 5.0 | 1.1 | 0.3 | 73.1 | 7.3 | 90.4 | 17.4 |
| S131Q | 80.6 | 8.6 | 18.9 | 3.8 | 82.8 | 15.6 | 106.2 | 16.2 |
| P132S | 104.8 | 9.2 | 3.2 | 0.6 | 107.3 | 18.7 | 103.7 | 0.5 |
| P132T | 93.1 | 13.4 | 11.7 | 1.3 | 97.6 | 8.2 | 88.9 | 6.8 |
| P132V | 86.9 | 4.7 | 84.6 | 6.8 | 107.5 | 7.8 | 107.4 | 6.7 |
| P132L | 89.6 | 4.8 | 6.1 | 1.0 | 86.5 | 9.2 | 105.4 | 9.3 |
| P132F | 68.5 | 8.8 | 1.6 | 0.4 | 54.1 | 3.1 | 86.2 | 11.3 |
| P132Y | 84.3 | 0.5 | 1.1 | 0.3 | 65.6 | 3.5 | 102.2 | 6.0 |
| P132H | 88.2 | 6.2 | 86.6 | 0.5 | 89.9 | | 93.8 | 9.9 |
| P132D | 90.5 | 10.0 | 87.6 | 3.0 | 84.6 | 6.1 | 101.7 | 1.4 |
| P132K | 97.1 | 1.1 | 2.2 | 0.5 | 83.2 | 12.1 | 95.8 | 14.7 |
| P132N | 100.7 | 4.4 | 2.2 | 0.5 | 89.8 | 11.8 | 98.6 | 0.5 |
| P132Q | 88.4 | 2.1 | 5.4 | 2.2 | 69.7 | 1.4 | 92.1 | 6.2 |
| L134T | 96.5 | 10.4 | 88.4 | 6.1 | 81.8 | 5.8 | 98.0 | 11.6 |
| L134I | 92.9 | 2.3 | 89.4 | 3.6 | 92.9 | 6.0 | 87.5 | 9.2 |
| L134Q | 62.2 | 2.1 | 47.1 | 2.4 | 66.7 | 1.2 | 69.8 | 10.3 |
| L134R | 5.0 | 1.0 | 1.0 | 0.3 | 2.3 | 0.7 | 1.9 | 0.2 |
| L134F | 101.7 | 8.3 | 90.7 | 10.5 | 69.2 | 4.7 | 97.8 | 2.0 |
| L134M | 86.1 | 9.6 | 99.5 | 14.0 | 95.0 | 17.8 | 105.3 | 7.7 |

### Example 7: Analysis and Comparison of the Identified Variants from the Comprehensive Analysis

Table 11 summarizes the variants that reduce binding of the tested antibodies to below 20%.

TABLE 11

| | | Antibodies that no longer |
|---|---|---|
| Residue | Mutation | bind to these mutations |
| D18 | R | Refmab #3 |
| P19 | D, K | Refmab #1 |
| | | Refmab #3 |
| N20 | G, S, T, L, P, Y, | Refmab #1 (not S and L) |
| | W, Q, D, E, K, V | Refmab #3 |
| F21 | S, V, L, I, H | Refmab #1 (not H) |
| | | Refmab #3 |
| W22 | G, S, T, L, P, Y, | Refmab #1 (only T, P E and R) |
| | N, Q, E, R | Refmab #3 |
| | | Refmab #6 |
| Q24 | S, T, L, H, E, P | Refmab #1 (only E) |
| | | Refmab #3 |
| T42 | P | Refmab #1 |
| | | Refmab #2 |
| | | Refmab #3 |
| | | Refmab #5 |
| | | Refmab #6 |
| | | Refmab #7 |
| | | Refmab #8 |
| F44 | S, V, I, L, P, Q, | Refmab #2 |
| | H, D, E, T | Refmab #3 (only, S, P and D) |
| | | Refmab #7 (only D and E) |
| | | Refmab #8 (only P, D and E) |
| H45 | Y | Refmab #3 |
| P46 | S, I, L, F, R, | Refmab #1 (not S, I and L) |
| | Y, H, D | Refmab #3 |
| | | Refmab #7 (only Y and D) |
| I47 | G, S, T, L, F, Y, | Refmab #2 |
| | W, Q, E, R, P, N | Refmab #3 (only P) |
| | | Refmab #5 (only P) |
| | | Refmab #7 (only Q, E, R, P and N) |
| | | Refmab #8 (only Q, E, R, P and N) |
| P48 | T, H, V, I, L, | Refmab #2 (only D) |
| | F, Y, M, W, N, | Refmab #4 (not V and I) |
| | Q, D, K, G, S | Refmab #5 (not T, V, M, K and S) |
| | | Refmab #7 |
| | | Refmab #8 (only N, D and S) |
| Y49 | P, Q, H, D, R, V, | Refmab #2 (only P, Q, H, D, G and C) |
| | G, C, S, T, I, L | Refmab #4 (only P, D, C, S and T) |
| | | Refmab #5 (not T, V, M, K and S) |
| | | Refmab #7 (not H and G) |
| | | Refmab #8 |
| Y50 | S, V, I, N, Q, H, | Refmab #4 (not P) |
| | D, R, G, L, P | Refmab #5 (not R and P) |
| | | Refmab #7 |
| D51 | K, Y | Refmab #2 (only K) |
| | | Refmab #5 (only K) |
| | | Refmab #7 |
| | | Refmab #8 |
| K52 | G, S, L, F, Y, | Refmab #1 (only F) |
| | D, E, H, T, I | Refmab #2 (only F and D) |
| | | Refmab #3 (only F) |
| | | Refmab #4 (only F and D) |
| | | Refmab #5 (not H) |
| | | Refmab #6 (only F) |
| | | Refmab #7 (only G, F, Y, D and E) |
| | | Refmab #8 |
| R122 | G, S, I, F, E, T | Refmab #2 |
| | | Refmab #7 |
| | | Refmab #8 (only I and F) |
| Y129 | S | Refmab #3 |
| S131 | K, F, M, W, Q | Refmab #6 |
| P132 | S, T, L, F, Y, | Refmab #6 |
| | K, N, Q | |
| L134 | R | Refmab #1 |
| | | Refmab #2 |
| | | Refmab #3 |
| | | Refmab #4 |
| | | Refmab #5 |
| | | Refmab #6 |
| | | Refmab #7 |
| | | Refmab #8 |

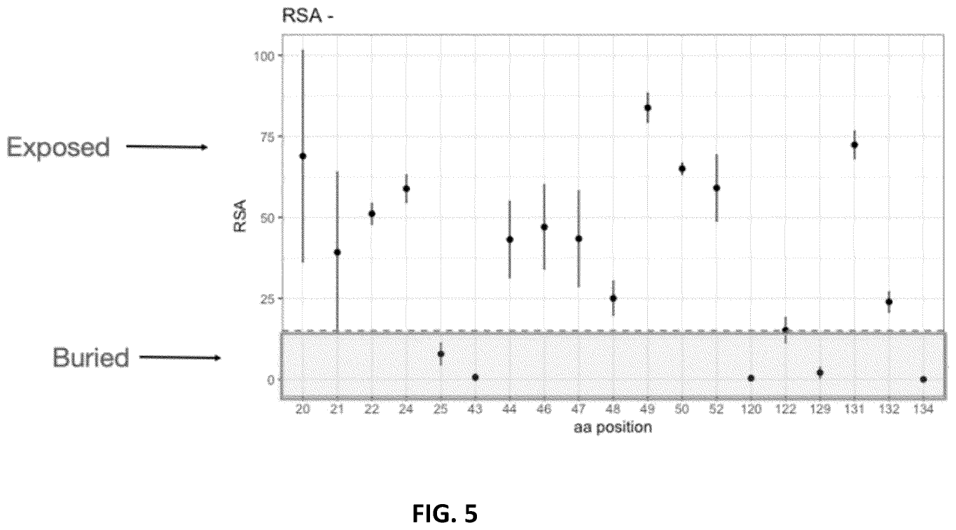

This analysis was also subject to additional validation, such as the assessment of the surface exposure of the respective residues. An exemplary analysis is shown in FIG. 5. It can be seen that certain residues are buried and hence not accessible on the protein surface. Such residues, including amino acids at positions 25, 43, 120, 129 and 134, were deselected.

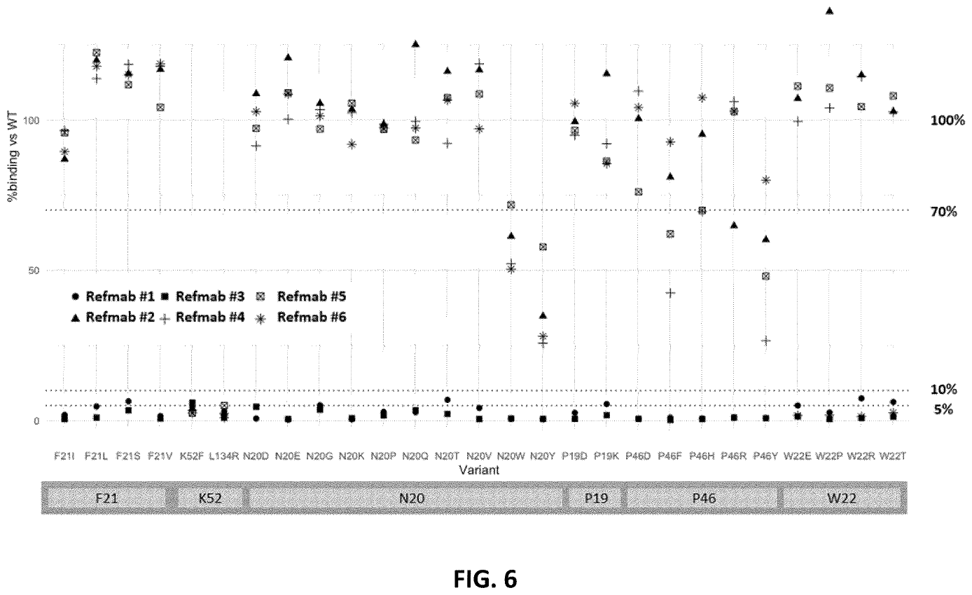

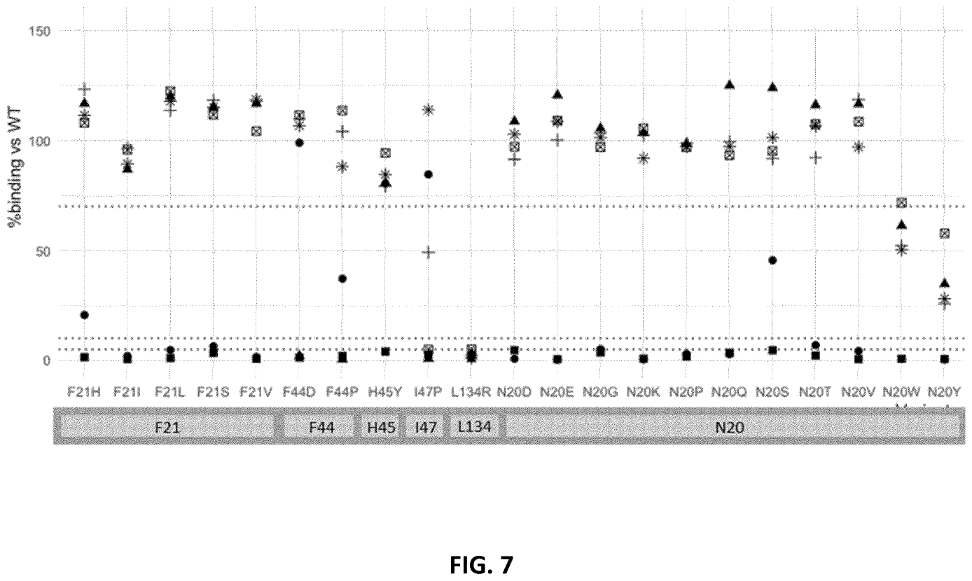

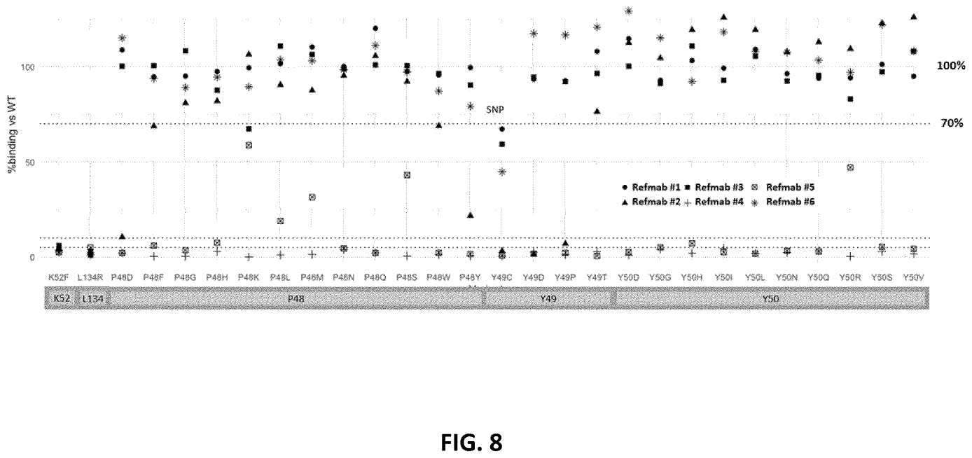

FIGS. 6-8 show the key CD33 variants that were identified to show a decreased binding of Refmab #1, Refmab #3 and Refmab #4, respectively, compared to wild-type CD33.

### Example 8: Binding of CD33 Variants to Refmab's

Analysis of binding to the elected variants was performed with antibodies Refmab #1, Refmab #3 and Refmab #4. Binding of the antibodies to CD33 wildtype and variants was measured in an Octet system R8 (Sartorius, SA-030159 and SA-030308) at 25° C. with shaking at 1,000 rpm using 1× kinetic buffer (Sartorius, PN: 18-1105). The elected variants were screened for their ability to bind Refmab #1, Refmab #3 and Refmab #4 using different concentrations of CD33 (wild type or variants). Antibodies were captured using Anti-Human Fc capture biosensor (AHC) (Sartorius, PN: 18-5060) for 300 s at 0.7 ug/mL. Human CD33 wt and variants, were used as analyte at 3 different concentrations (500, 50 and 5 nM). Association of the analyte to the antibodies was monitored for 700 s and dissociation of the analyte from the antibodies was monitored for 1200 s. Reference subtraction was performed against buffer only wells. AHC tips were regenerated using 10 mM Glycine-HCl pH 1.7. Data were analyzed with Octet Data Analysis software HT 12.0. Data were fitted to a 1:1 binding model. Kinetic rates ka and kd and response signal were globally fitted. The binding level of each CD33 variants to Refmab #1, Refmab #3 and Refmab #4 was calculated by normalizing the response at the end of the association by the loading, this value was compared to the wild type and shown as percentage. Variant N20R binds unspecifically to the capture biosensor. Results are shown in Table 12.

$$\% binding mutant A = 100 * \frac{response mutant A nm}{loading response RefmAb X nm} / \frac{response WT nm}{loading response RefmAb X nm}$$

TABLE 12

| | Refmab #1 | Refmab #3 | Refmab #4 |
|---|---|---|---|
| | % WT binding | % WT binding | % WT binding |
| WT | 100.00 | 100.00 | 100.00 |
| N20G | 0.00 | 0.00 | 115.33 |
| N20S | 37.79 | 34.37 | 84.12 |
| N20D | 0.00 | 0.00 | 91.33 |
| N20E | 3.46 | 6.04 | 100.21 |
| N20K | 0.00 | 0.00 | 102.49 |
| N20V | 18.64 | 6.34 | 96.48 |
| N20R | Unspecific | Unspecific | Unspecific |
| N20H | 67 | 0 | 104 |
| F21V | 0.00 | 0.00 | 100.76 |
| F21L | 1.52 | 0.00 | 109.05 |
| F21I | 0.00 | 0.00 | 84.22 |
| F21S | 0.00 | 0.00 | 78.30 |
| F21M | 0.00 | 0.00 | 80.00 |
| F21N | 0.00 | 0.00 | 83.00 |
| F21Q | 0.00 | 0.00 | 89.00 |
| F21H | 0.00 | 0.00 | 108.00 |
| W22S | 45.85 | 20.06 | 92.95 |
| W22T | 14.98 | 3.39 | 87.87 |
| W22E | 26.36 | 0.00 | 82.79 |
| W22R | 26.36 | 0.00 | 82.79 |
| W22D | 0 | 0 | 104 |
| W22K | 0 | 0 | 113 |
| W22H | 106 | 55 | 112 |
| Q24R | 67.86 | 88.38 | 101.90 |
| F43S | 104.03 | 64.80 | 80.00 |
| F44P | 0.00 | 0.00 | 87.36 |
| F44S | 63.92 | 39.00 | 84.60 |
| H45Y | 55.00 | 35.13 | 69.28 |
| P48F | 76.17 | 74.30 | 9.11 |
| P48D | 71.87 | 76.38 | 5.75 |
| P48S | 79.52 | 73.95 | 21.84 |
| Y50S | 75.47 | 70.94 | 22.58 |
| Y50D | 71.54 | 71.05 | 0.00 |
| Y50R | 73.31 | 70.45 | 0.00 |
| Y50L | 76.06 | 74.43 | 25.34 |
| Y50E | 101.00 | 120.00 | 16.00 |
| Y50K | 101.00 | 122.00 | 0.00 |
| Y50H | 104.00 | 111.00 | 28.00 |
| Y50H_Y49H | 76.39 | 84.43 | 0.00 |
| N100Q | 107.00 | 124.00 | 114.00 |
| R119A | 121 | 118 | 95 |
| F21S_W22R | 0 | 0 | 109.7 |

### Example 9: Avidity Binding of CD33 Variants to Refmab's

Analysis of binding to the elected variants was performed with antibodies Refmab #1, Refmab #3 and Refmab #4. Binding of the antibodies to CD33 wildtype and variants was measured in an Octet system R8 (Sartorius, SA-030159 and SA-030308) at 25° C. with shaking at 1,000 rpm using 1× kinetic buffer (Sartorius, PN: 18-1105). The elected variants were screened for their ability to bind Refmab #1, Refmab #3 and Refmab #4.

CD33 wild type and variants were biotinylated with EZ-Link™ Sulfo-NHS-LC-Biotin, No-Weigh (Thermo Fisher, A39257). Biotinylated CD33 wild type and variants were captured on streptavidin biosensor (SA) (Sartorius, PN 18-5019) for 300 s at 2 to 16 μg/mL. As an analyte, Refmab #1 was used at 300 nM or 500 nM, Refmab #3 and Refmab #4 were used at 500 nM. Association of the analyte was monitored for 300 s and dissociation was monitored for 900 s. Data were analyzed using the Octet Data Analysis software HT 12.0. Binding level of each CD33 variants to Refmab #1, Refmab #3 and Refmab #4 was calculated by subtracting the response signal at 1080s by the loading, this value was compared to the wild type and shown as percentage. Results are shown in Table 13.

$$\% binding mutant A = 100 * Response at 1080 s RefmAb X - loading response mutant A at 800 s / Response at 1080 s RefmAb X - loading response WT \text{?}$$

$$\text{?} \text{indicates text missing or illegible when filed}$$

TABLE 13

| | Refmab #1 | Refmab #3 | Refmab #4 |
|---|---|---|---|
| | % WT binding | % WT binding | % WT binding |
| WT | 100.00 | 100.00 | 100.00 |
| N20G | 9.44 | 0.00 | 88.96 |
| N20E | 5.59 | 0.00 | 111.36 |
| N20K | 6.79 | 0.00 | 79.40 |
| N20V | 29.26 | 6.62 | 99.53 |
| N20H | 32.91 | 5.39 | 87.69 |
| F21V | 5.41 | 0.00 | 95.96 |
| F21L | 12.21 | 0.00 | 88.28 |
| F21I | 0.00 | 0.00 | 91.00 |
| F21S | 9.03 | 0.00 | 106.20 |
| F21M | 6.62 | 0.00 | 122.09 |
| F21N | 0.00 | 0.00 | 108.48 |
| F21Q | 0.00 | 0.00 | 122.12 |
| F21H | 10.45 | 0.00 | 103.22 |
| W22T | 37.47 | 5.25 | 99.98 |
| W22E | 67.52 | 23.54 | 135.75 |
| W22R | 51.91 | 0.86 | 87.22 |
| W22D | 12.68 | 6.42 | 129.03 |
| W22K | 22.38 | 0.00 | 99.33 |
| F44P | 2.00 | 0.00 | 73.90 |
| Y50D | 131.26 | 128.26 | 0.00 |
| Y50R | 107.91 | 93.13 | 0.00 |
| Y50K | 86.57 | 73.91 | 0.00 |
| Y50H_H49H | 95.80 | 58.50 | 0.00 |

### Example 10: Generation of CD33 Variants by Base Editing in Primary Human T Cells

Certain CD33 variants were generated via base editing. To do so, we used ABE8e-NG, a published base editor (Nat Biotech (2020) 38: 883-91) to target selected regions of CD33. The variants generated and the sgRNAs used are shown in Table 14.

For base editing, 1.5 μg of the base editor (ABE8e-NG mRNA; ordered from Trilink) and 1.5 μg of sgRNA (ordered from Synthego) were electroporated into 187,500 human activated T cell isolated from PBMCs using an EasySep Human T Cell Isolation Kit (Stemcell Technologies #17951). 24 h after isolation, human T cells were activated for 48 h at a concentration of 1.5 million cells per mL with IL-2 (150 U/mL), II-7 (5 ng/mL), IL-17 (5 ng/mL) and Dynabead Human T-Activator CD3/CD28 (Gibco #11132D) following the manufacturer's recommendation (1:1 beads:cells ratio) in a 96-well plate. Human activated T cells were de-beaded and for each condition, 187,500 cells were mixed with 1.5 μg of ABE8e-NG mRNA, 1.5 μg of sgRNA, 8.2 μL of P3 primary and 1.8 uL of supplement 1 in small shuttle nucleo-cuvettes and electroporated with a Lonza 4D-Nucleofector using the EH-115 pulse program. Immediately after electroporation, 90 μL of pre-warmed human T cells medium (RPMI-1640 (Sigma-Aldrich #R8758-500ML)+10% heat-inactivated human serum (male, AB+)+100× Na-Pyruvate (Gibco #11360-039)+100× GlutaMAX(ThermoFisher Scientific #35050061)+Beta-Mercaptoethanol (Gibco #31350-010)+HEPES (Sigma #H0887)+100×MEM non-essential amino acids (Gibco #11140-050)+1% Penicillin+Streptomycin (Gibco #15140-122) were added directly in the nucleo-cuvettes and incubated for 20 minutes at 37° C. for cells to recover. Electroporated conditions were then transferred in 96 well flat-bottom plates with addition of 500 U/mL of IL-2. Medium was renewed every 48 hours. 5 days post-electroporation, bulk cells of each condition were spun down for 5 min at 2200 RCF. Pellets were resuspended in 30 μL of QuickExtract solution (Lucigen #QE09050) in PCR tubes and were shaken for 1 minute with a vortex. We incubated mixes at 60° C. in a thermoblock for 6 minutes. Lysates were thoroughly mixed a second time during 1 minute with a vortex and re-incubated at 98° C. for 10 minutes. After concentration measurements, final lysates were PCR-ready. gDNA of each condition was subjected to two PCR reactions: (1) with primers Fwd-1 and Rev-1 to amplify across CD33 and (2) with primers Fwd-2 and Rev-2 to amplify across pseudogene SIGLEC22P (Table 14). The pseudogene SIGLEC22P is located 13.5 kb upstream of CD33 and shares over 90% sequence similarity to the first three exons of CD33. It was therefore also evaluated if base editing led to an, undesired, change in the SIGLEC22P pseudogene.

TABLE 14

| Primer | Sequence (5′-3′) | Target | Amplicon Size |
|---|---|---|---|
| Fwd-1 | TAAACACCCCATGGATCTAGG (SEQ ID No. 48) | CD33 | 952 bp |
| Rev-1 | GCAGTACCCATGAACTTCCC (SEQ ID No. 49) | | |
| Fwd-2 | CCACAGGTCAAGAAGAGGCC (SEQ ID No. 50) | SIGLEC22P | 937 bp |
| Rev-2 | TTGACCTTCCCTTGTGGCTG (SEQ ID No. 51) | | |

PCR products were column-purified and sequenced by Sanger Sequencing (Microsynth) using primer Fwd-1 for the CD33 amplicon and primer Fwd-2 for the SIGLEC22P amplicon, respectively.

Base editing efficiencies were quantified from Sanger sequencing reads using EditR (CRISPR J. (2018) 1:239-250).

Results are summarized in Tables 15 and 16. No editing could be observed for residues D18 and N20. Some sgRNA's led to an editing efficiency between 10-50% (F21, F44_1), others to an editing efficiency of >80% (Q244, F44_2, H45, Y49, Y50). Some sgRNA's led to the modification of bystanders at various efficiencies. Some sgRNA's (F44_1) led to silent bystanders (nucleotide changes do not change the amino acid sequence). Some sgRNA's also let to editing of SIGLEC22P. In summary, it could be shown that most of the variants investigated are amenable to base editing. Experimental conditions need to be optimized, which is a matter of routine experimentation.

TABLE 15

| | Codon | Target | | | | |
|---|---|---|---|---|---|---|
| | (SEQ ID | amino | | | sgRNA sequence 5′-3′ | PAM (SEQ |
| Residue | No.) | acid | Strand | sgRNA ID | (SEQ ID No.) | ID No.) |
| D18 | GAT (52) | G | + | sgCIT06_D18_1 | TATGGATCCAAATTTCTGGC (53) | TGC (54) |
| N20 | AAT (55) | D/S/G | + | sgCIT06_N20_1 | TCCAAATTTCTGGCTGCAAG (56) | TGC (54) |
| | | | | sgCIT06_N20_2 | AAATTTCTGGCTGCAAGTGC (57) | AGG (59) |
| | | | | sgCIT06_N20_3 | GATCCAAATTTCTGGCTGCA (58) | AGT (60) |
| F21 | TTC (61) | L/S/P | − | sgCIT06_F21_1 | AGAAATTTGGATCCATAGCC (62) | AGG (59) |
| | | | | sgCIT06_F21_2 | AGCCAGAAATTTGGATCCAT (63) | AGC (64) |
| W22 | TGG (65) | R | − | sgCIT06_F21_2 | AGCCAGAAATTTGGATCCAT (63) | AGC (64) |
| Q24 | CAA (66) | R/Q | + | sgCIT06_Q24_1 | GCTGCAAGTGCAGGAGTCAG (67) | TGA (69) |
| | | | | sgCIT06_Q24_2 | TGGCTGCAAGTGCAGGAGTC (68) | AGT (60) |
| F44 | TTC (61) | L/S/P | − | sgCIT06_F44_1 | TGGGATGGAAGAAAGTGCAG (70) | GGC (72) |
| | | | | sgCIT06_F44_2 | ATGGAAGAAAGTGCAGGGCA (71) | CGA (73) |
| H45 | CAT (74) | R | + | sgCIT06_H45_1 | TTCCATCCCATACCCTACTA (75) | CGA (73) |
| Y49 | TAC (76) | H | − | sgCIT06_Y49_1 | GTAGTAGGGTATGGGATGGA (77) | AGA (78) |
| Y50 | TAC (76) | C | + | sgCIT06_Y50_1 | TACTACGACAAGAACTCCCC (79) | AGT (60) |
| | | H | − | sgCIT06_Y50_2 | TGTCGTAGTAGGGTATGGGA (80) | TGG (65) |
| | | | | sgCIT06_Y50_3 | GTCGTAGTAGGGTATGGGAT (81) | GGA (82) |

TABLE 16

| | | On target editing | Amino acid | Bystander editing | Amino acid | |
|---|---|---|---|---|---|---|
| Residue | sgRNA ID | efficiency | change | efficiency | change | SIGLEC22P |
| D18 | sgCIT06_D18_1 | ND | ND | ND | ND | ND |
| N20 | sgCIT06_N20_1 | ND | ND | ND | ND | ND |
| | sgCIT06_N20_2 | ND | ND | ND | ND | ND |
| | sgCIT06_N20_3 | sequencing failed | | | | |
| F21 | sgCIT06_F21_1 | 36% A⁴ > G | F21L | 43% A⁵ > G | N20N | ND |
| | sgCIT06_F21_2 | 20% A⁷ > G | F21S | ND | ND | ND |
| Q24 | sgCIT06_Q24_1 | 100% A⁶ > G, 48% A⁷ > G | Q24R | ND | ND | 100% A⁶ > G, 52% A⁷ > G |
| | sgCIT06_Q24_2 | 100% A⁸ > G | Q24R | ND | ND | 100% A⁸ > G |
| F44 | sgCIT06_F44_1 | 10% A⁹ > G | F44S | 100% A⁵ > G | H45H | 42% A⁵ > G |
| | sgCIT06_F44_2 | 100% A⁵ > G, 100% A⁶ > G | F44P | 80% A⁸ > G | F43S | 100% A⁶ > G, 56% A⁸ > G |
| H45 | sgCIT06_H45_1 | 91% A⁵ > G | H45R | 20% A¹⁰ > G, 36% A¹² > G | I47V/M | 35% A⁵ > G |
| Y49 | sgCIT06_Y49_1 | 87% A⁶ > G | Y49H | 58% A³ > G | Y50H | ND |
| | | | | 68% A¹¹ > G | I47T | |
| Y50 | sgCIT06_Y50_1 | 100% A⁵ > G | Y50C | 54% A⁸ > G | D51G | ND |
| | | | | 24% A¹⁰ > G | K52E | |
| | sgCIT06_Y50_2 | 100% A⁷ > G | Y50H | 92% A¹⁰ > G | Y49H | 100% A⁷ > G, 65% A¹⁰ > G |
| | sgCIT06_Y50_3 | 100% A⁶ > G | Y50H | 92% A⁹ > G | Y49H | 100% A⁶ > G, 78% A⁹ > G |

### Example 11: Base-Edited CD33 Variants of Primary CD34-Positive Human Cells Show a Loss of Binding to Anti-CD33 Antibodies

For base editing of CD33 leukopaks were purchased from CytoCare and hCD34+ HSPCs were isolated by the LP-34 process using the CliniMACS Prodigy (Miltenyi). hCD34+ HSPCs were thawed and resuspended in pre-warmed HSPC medium (StemSpan SFEM II (StemCell #09655)+1 μL/mL hSCF (Miltenyi #130-096-695)+1 μL/mL hFlt3-Ligand (Miltenyi #130-096-479)+1 μL/mL hTPO (Miltenyi #130-095-752). After two days of culture, per base editing condition, 1 million hCD34+ HSPCs were mixed with 7.5 μg of a SpRy Cas9 variant (Science 2020 (368: 190-6); TebuBio), 13.6 μg of sgRNA (Synthego; 1:100 BE:sgRNA molar ratio), 82 μL of P3 primary and 18 μuL of supplement 1 in large-sized nucleo-cuvettes (Lonza #V4XP-3024) and electroporated with a Lonza 4D-Nucleofector using the Lonza CA-137 pulse program. Alternatively, 200,000 hCD34+ HSPCs cells were used with appropriate scaling of the other components. For this scale nucleo-cuvettes were used (Lonza #V4XP-3032). Immediately after electroporation, 900 or 80 μL of pre-warmed stem cell medium was added directly in the nucleo-cuvettes and they were incubated for 20 minutes at 37° C. for cells to recover. Electroporated hCD34+ HSPCs were kept in culture at 500,000 cells per mL in 6-well or 48-well flat-bottom plate in stem cell medium supplemented with hSCF, hFLT3-Ligand and hTPO (see above) renewed every 5 days. gDNA from bulk cells was sequenced 5 days post-electroporation to assess base editing efficiencies.

In order to generate W22R variant of CD33 by base editing, 21 different sgRNA's were tested.

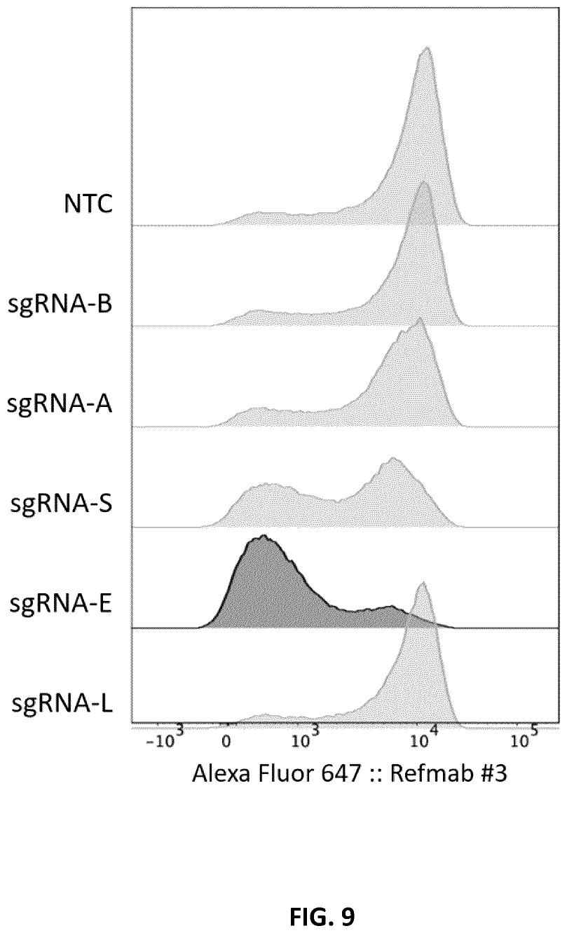

Histograms of binding to Refmab #3 after base editing of human CD34+ HSPC with representative sgRNAs in comparison to non-target control (NTC) are shown in FIG. 9. It can be seen that the majority of cells were edited with sgRNA-E. Sequences of the sgRNAs used are shown in Table 17:

| sgRNA | Nucleic acid sequence | SEQ ID No. |
|---|---|---|
| NTC | GCACTACCAGAGCTAACTCA | 83 |
| sgRNA-B | TTGCAGCCAGAAATTTGGAT | 84 |
| sgRNA-A | TGCAGCCAGAAATTTGGATC | 85 |
| sgRNA-S | GCAGCCAGAAATTTGGATCC | 86 |
| sgRNA-E | CAGCCAGAAATTTGGATCCA | 87 |
| sgRNA-L | AGCCAGAAATTTGGATCCAT | 88 |

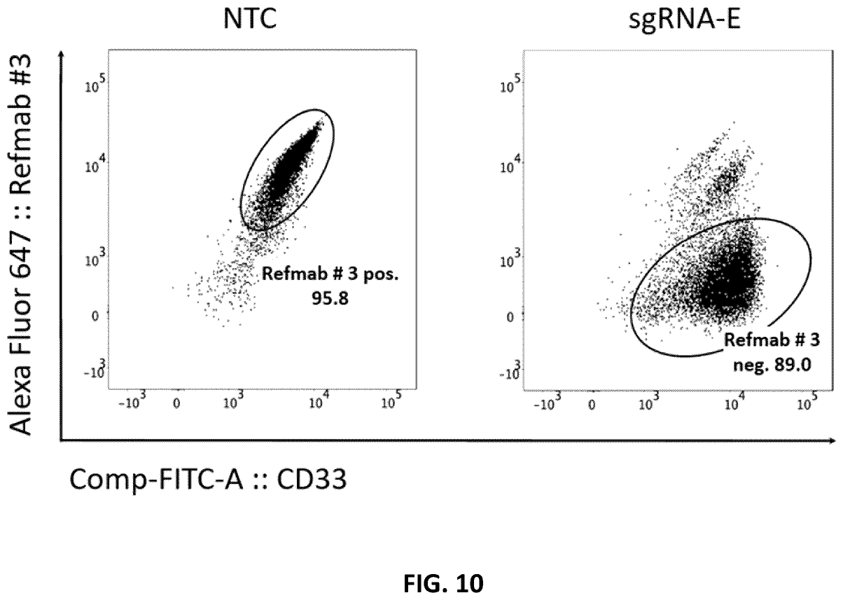

Edited human HSPCs were tested for binding to Refmab #3 and a control antibody by flow cytometry. Cells were washed with PBS, then a viability staining was performed with eFluor780 (dilution 1:1000, eBioscience, #65-0865-14). After an additional washing step, cells were labelled with Refmab #3 with AF647 and FITC-HIM3-4 (dilution 1:25, BioLegend, #303304). Results are shown in FIG. 10 for sgRNA-E. It can be seen that 95.8% of the non-transformed control cell did react with Refmab #3, whereas 89.0% of the cells transfected with sgRNA-E did not react with Refmab #3. Binding to FITC HIM 3-4 is unaffected by the editing.

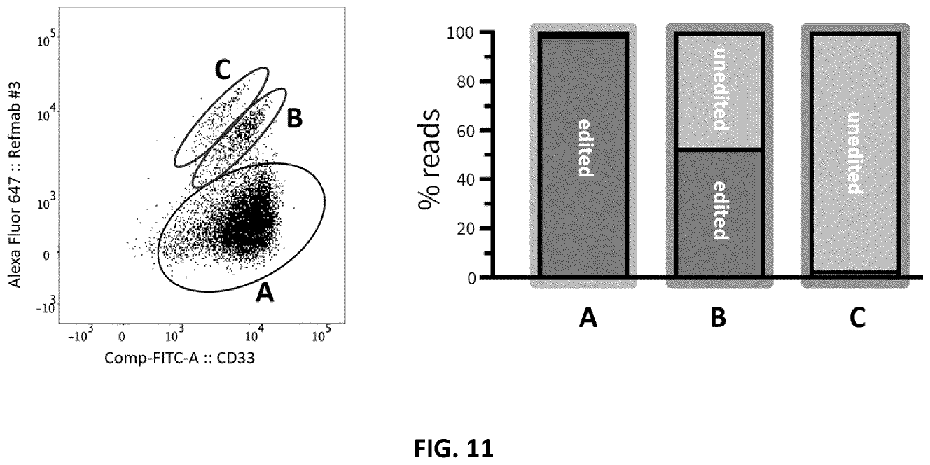

To further confirm that successful base editing is linked to a loss of binding to Refmab #3, NGS was performed on sorted cells. Results are shown in FIG. 11. Essentially all cells which lost binding to Refmab #3 (A) show homozygous editing (target edit/target edit), whereas essentially all cells reactive to Refmab #3 (C) did show homozygous wild type sequence. The intermediate cell population (B) showed heterozygous editing (WT/target edit).

### Example 12: Base-Edited CD33 Variants of Primary CD34-Positive Human Cells are Functionally Indistinguishable from Unedited Cells

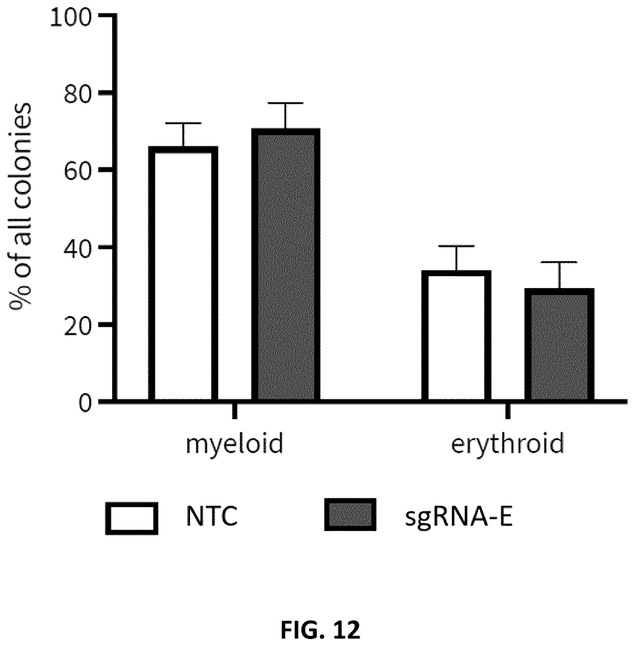

Human CD34-positive HSPCs that were edited with sgRNAs NTC (control) and sgRNA-E (variant W22R) were tested in a colony-forming unit (CFU) assay as follows: sgRNA-E and NTC (control) edited HSC were washed and resuspended in IMDM 2% FBS medium (200 cells/well) and added to MethoCult (Stemcell #044349). 1 ml of the suspension is then carefully dispensed into a plate (Stemcell SmartDish™) and incubated at 37° C. for 14 days. Then images were taken and colonies were quantified. Results are shown in FIG. 12.

As can be seen cells differentiated to the same degree into myeloid cell and erythroid cells, respectively, demonstrating that gene editing had no impact on the differentiation of cells.

Sanger sequencing showed that about 65% of HSPCs edited with sgRNA-E showed homozygous edits, and about 30% show heterozygous edits. The remaining cells could not clearly be grouped.

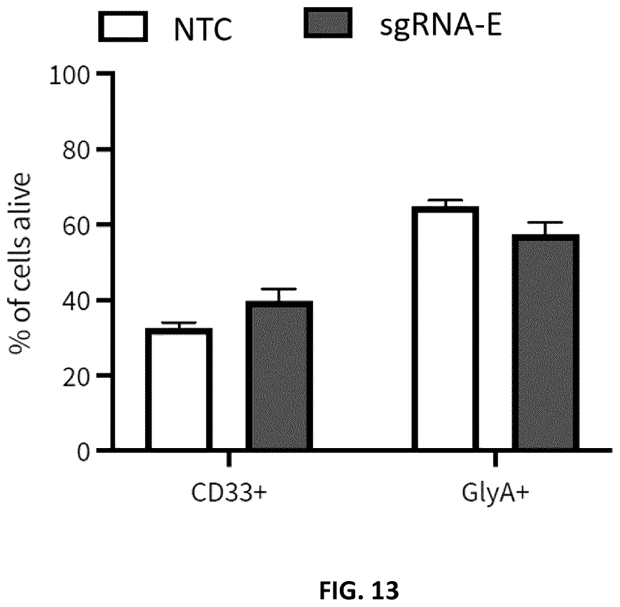

To further confirm, that gene editing had no impact on the function of the edited HSPCs, HSPCs that were edited with sgRNAs NTC (control) and sgRNA-E (variant W22R) were tested in an in vitro differentiation assay. In this assay, 2000 cells each were seeded in a 96-well plate in 100 μl StemPor-34 SFM Kit (Gibco, #10639-011) supplemented with 1% L-Glutamine, 1% Penicillin and a cytokine mix (Flt3 ligand (20 ng/μl, Miltenyi biotech #130-096-479), TPO (50 ng/μl, Milteny biotech #130-095-752), SCF 100 ng/μl (Milteny biotech #130-096-695), 1L3 (10 ng/μl, Milteny biotech #130-095-071), 1L6 (50 ng/μl, Milteny biotech #130-093-934), EPO (3 ng/μl, Stemcell Technologies #78007), 1L2 (10 ng/μl, Milteny biotech #130-097-743), IL7 (20 ng/μl, Milteny biotech #130-095-743), GM-CSF (20 ng/μl, Fisher #PHC2015), human LDL (500 ng/ml, Stemcell Technologies, #2698). After 4 days, additional 100 μl of cytokine medium was added, cells were split after 8 and 10 days. Differentiation of cells was assessed after 14 days via FACS. Results are shown in FIG. 13. As can be seen no difference in the differentiation could be shown.

### Example 13: Introduction of CD33 Variants by Gene Editing Renders Human CD34+ Cells Resistant to Killing by Refmabs Conjugated to a Toxin

Human CD34+ cells are gene edited as described above using base editing, prime editing or HDR to introduce the relevant variants. The edited CD34+ cells are used as either bulk cells or sorted to enrich edited cells carrying the respective CD33 variant.

Approximately 10³ to 10⁴ engineered and sorted human CD34+ cells are seeded in 96 well plates in 100 μl of medium supplemented with cytokines hSCF, hFLT3L and hTPO. Refmabs are directly coupled to a toxic payload such as PBD/tesirine or SN38. Cells are incubated for 3-5 days at 37° C. with serial dilutions of the Refmab antibody drug conjugate (ADC). At the end of the incubation period, 100 μl of CellTiter Glow (Promega Cat Nr: G9241) is added to each well. Luminescence is read with an integration time of 1 s. Unedited CD34+ cells expressing wildtype CD33 cells serve as positive control for maximum killing.

Alternatively, edited bulk CD34+ cells are incubated for 3-5 days at 37° C. with serial dilutions of the Refmab antibody drug conjugate (ADC). After incubation, the cells are collected, stained with the appropriate fluorescent labeled Refmabs and resuspended in 200 μL of FACS buffer for flow cytometry analysis. The cell suspension is then analyzed using a flow cytometer. Wildtype CD33 cells bind both CD33 antibodies, whereas edited, shielded CD33 cells bind only the irrelevant antibody/Refmab. Compared to the PBS control (no ADC), depletion of Refmab-positive cells is observed when the antibody-toxin is added. Moreover, Refmab negative cells persist and are shielded from the ADC mediated killing.

### Example 14: Gene Editing of CD33+ Human Cell Line Shields from Antibody Binding and Killing

For base editing of CD33, human myeloid cell lines such as U937, KG-1 or THP-1 are electroporated with ABE8e-NG mRNA (TriLink) and sgRNA (Synthego) following the manufacturer's recommendations. Electroporated cells are expanded for 5 days with media renewal every 48 hours.

CD33 variants which cannot be introduced by base editing are generated by homology-directed repair (HDR) and/or primed editing of human CD34+ cells, using appropriate tools. Binding and killing assays are performed as described below.

CD33 edited U937 and THP-1 cells are sorted by flow cytometry to generated homogenously edited pools or single cell clones.

To assess if gene edited human cells are shielded from antibody binding cells are collected, stained with the appropriate fluorescent-labeled Refmabs and anti-CD33 antibodies and resuspended in 200 μL of FACS buffer for flow cytometry analysis. The cell suspensions are analyzed using a cell analyzer (flow cytometer). Compared to the isotype control, edited and shielded cells will bind an irrelevant Refmab/anti-CD33 antibody, but not the Refmab paired with the shielding mutation. Wildtype, unedited cells will bind both antibodies. The irrelevant RefMab/CD33 antibody serves as an expression control for CD33.

In order to see if gene edited human cells are shielded from antibody-toxin conjugate depletion, gene edited, sorted cells are incubated for 3-5 days in 100 μL of human medium with addition of increasing concentrations of Refmab-based antibody-drug conjugates. At the end of the incubation period, 100 μl of CellTiter Glow (Promega Cat Nr: G9241) is added to each well. Luminescence is read with an integration time of 1 s. Unedited cells expressing wildtype CD33 as well as CD33-negative cells serve as positive (maximum killing) and negative (background killing) controls.

Compared to the PBS control, depletion of Refmab-positive cells is observed when the antibody-toxin is added. Moreover, Refmab negative cells remain present and are shielded from the Antibody-toxin mediated killing.

Variants which cannot be introduced by base editing will be generated by homology-directed repair and/or primed editing using appropriate tools. Binding and killing assays are performed as described above.

### Example 15: Overexpression of Recombinant CD33 Variants in Cell Lines

Mammalian cell lines such as HEK293 or chicken DF-1 cells, which show expression of endogenous CD33 are suitable for the overexpression of recombinant human wildtype or mutant CD33 variants. Cells are transfected with a construct expressing wildtype CD33 or with constructs expressing mutant CD33 variants. Two to three days after transfection cells are selected with antibiotics (G418/Neomycin) in order to generate stable cell lines. Alternatively, the transient CD33 expressing cells can also be used directly for FACS analysis. Transient cells or stable cells lines are stained using 2 different human CD33 antibodies. One antibody binds to the region of the mutation and the second antibody binds a different region. Using this antibody combination, it is possible to evaluate the loss of binding of the antibody of interest to a specific, paired CD33 mutation. The retention of binding of the second antibody is important to show that the mutant CD33 is still expressed by the cells.

### Example 16: Binding of CD33 Wildtype and Variants to Sialic Acid Ligands

Analysis of the elected variants functionality was assessed through the binding to a sialic acid in an ELISA assay. MaxiSorp plates (Thermo Fisher 43954) were coated overnight with CD33 wildtype and CD33 variants at 30 ng/well and blocked with 2% Bovin Serum Albumin. Biotin-conjugated polyacrilamide probe substituted with Neu5Aca3′Lac-Gly (Neu5Acα2-3Galβ1-4Glcβ-sp4-PAA-biot “5.3SLL”, GlycoNZ, 0060-BP) or Neu5Acα6′Lac-C2 ((Neu5Acα2-6Galβ1-4Glcβ-sp2-PAA-biot “5.6SLL”, GlycoNZ, 0063a-BP) were incubated 2 h at 250 ng/well and washed three times. Binding signal was detected using 50 uL/well of streptavidin HRP (Biotechne DY998) diluted 200-fold for 30 min, followed by adding 50 uL/well of 3,3′,5,5′-Tetramethylbenzidine (TMB, Thermo Fisher 002023) and incubation for 10 min. Reaction was stopped with Stop Solution (Thermo Fisher N600). Absorbances at 450 nm was measured for binding signal and 570 nm for background. Samples were compared using an unpaired T-test.

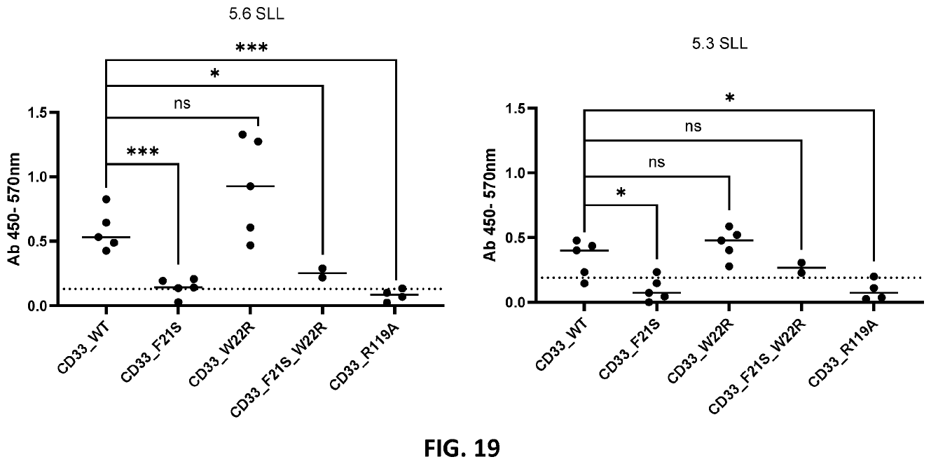

Results are depicted in FIG. 19. CD33 R119A does not bind sialic acid, it is a negative control variant, as the R119 residue is conserved among the Siglec family and plays an important role for the binding to sialic acid. CD33 wildtype and W22R variant are binding to sialic acid, stronger to Neu5Acα6′Lac-C2 compared to Neu5Acα3′Lac-Gly suggesting a preference to 5.6 linkage over 5.3 linkage. W22R mutation has no significant impact on the binding to sialic acid compared to wildtype. F21S mutant does not bind sialic acid or much weakly compared to wildtype. Double variant F21S_W22R binds weakly to sialic acid, stronger than F21S and weaker than W22R.

### Example 17: Inhibition of Phagocytosis by CD33 Wildtype and Variants

CD33 is described to be an anti-phagocytic receptor, restricting phagocytosis of cells like, e.g., human microglia and macrophages. As expected, the knock-out or functional blockade of human CD33 result in increased phagocytosis, thus the functional activity of CD33 variants can be assessed by quantifying the phagocytic activity of wildtype CD33 versus variant CD33 cells. Human myeloid cell lines such as THP-1 and U937 are used to assess CD33 mediated phagocytosis.

Therefore, CD33 variants are introduced into the genome of THP-1 and U937 cells using the methods described above and sorted to generated homogenously edited pools or single cell clones (see also above).

Prior to the assay, wildtype and gene edited variant CD33 THP-1 and U937 cells are cultivated, harvested, centrifuged, resuspended in medium, and seeded in 96-well plates. Fluorescent cargo, such as fluorescent polystyrene beads, pHrodo bioparticles (E. coli, zymosan), tetramethylrhodamine isocyanate-dextran, pHrodo-labelled myelin, fluorescent-labelled amyloid beta and others are added to the cells and incubated for 30 mins to 4 hrs at 37° C. For the bead-based cargo, the % of cells taking up at least one bead is used for quantification in a flow cytometer, whereas for non-bead-based cargo the extent of phagocytosis is determined by assessing the median fluorescence intensity (MFI) of the fluorescent signal with a background subtraction (cells without cargo). Alternatively, real-time monitoring and quantification of phagocytosis can be performed using the Incucyte device.

### Example 18: CD33 Variants and Refmab #1

Among all the variants tested amino acid residues N20, F21 and W22 seem to be particularly crucial for the binding of Refmab #1 to CD33. Mutations of these residues lead to a strong decrease of binding. Among these three residues, N20 and F21 are particularly important.

As for N20, certain amino acid exchanges seem to be particularly useful for inhibiting the binding of Refmab #1 to CD33. This includes substitutions N20G, N20D, N20E, N20K, N20H and N20R. Among these variants N20G and N20K are preferred variants, also due to their favorable yields in production and biophysical properties, which are essentially indistinguishable from wild-type CD33.

Also for F21, certain amino acid exchanges seem to be particular useful for inhibiting the binding of Refmab #1 to CD33. This includes substitutions F21V, F21L, F211, F21S, F21M, F21N, F21Q and F21H. Among these variants F21V, F21L, F211 and F21S are preferred variants, also due to their favorable yields in production and biophysical properties, which are essentially indistinguishable from wild-type CD33. Variants F21V, F21S and F211 are most preferred due to their favorable aggregation behavior.

### Example 19: CD33 Variants and Refmab #3

Among all the variants tested amino acid residues N20, F21 and W22 seem to be particularly crucial for the binding of Refmab #3 to CD33. Mutations of these residues leads to a strong decrease of binding. Among these three residues, N20 and F21 are particularly important.

As for N20, certain amino acid exchanges seem to be particular useful for inhibiting the binding of Refmab #3 to CD33. This includes substitutions N20G, N20E, N20D, N20R, N20H and N20K. Among these variants N20G, N20K and N20D are preferred variants, also due to their favorable yields in production and biophysical properties which are essentially indistinguishable from wild-type CD33. Even more preferred are N20K and N20D due to their lower aggregation propensity.

Also for F21, certain amino acid exchanges seem to be particular useful for inhibiting the binding of Refmab #3 to CD33. This includes substitutions F21V, F21L, F211, F21M, F21N, F21Q, F21H and F21S. Among these variants F211, F21V, F21L and F21S are preferred variants, also due to their favorable yields in production and biophysical properties which are essentially indistinguishable from wild-type CD33. Even more preferred are F211, F21V and F21S due to their lower aggregation propensity.

For W22, certain amino acid exchanges seem to be particularly useful for inhibiting the binding of Refmab #3 to CD33. This includes substitutions W22R, W22D, W22K, W22H and W22T. Among these variants W22R is a preferred variant, also due to their favorable yields in production and biophysical properties which are essentially indistinguishable from wild-type CD33. W22T has good production yield, biophysical properties and shielding from Refmab #3, but it will introduce a potential N-linked glycosylation site which could change the glycosylation pattern of CD33 and is therefore not preferred.

### Example 20: CD33 Variants and Refmab #4

Among all the variants tested amino acid residue Y50 seems to be particularly crucial for the binding of Refmab #4 to CD33. Mutations of this residue lead to a strong decrease of binding.

Certain amino acid exchanges seem to be particularly useful for inhibiting the binding of Refmab #4 to CD33. This includes substitutions Y50D, Y50E, Y50K, Y50H and Y50R. Among these variants Y50D and Y50R are preferred variants, also due to their favorable yields in production and biophysical properties, which are essentially indistinguishable from wild-type CD33.

### Example 21: Biophysical Properties of CD33 Variants

Low aggregation propensity and high melting temperature are characteristics of the structural integrity and stability of biomolecules, such as the extracellular domains of protein receptors. Therefore, the extracellular region of wildtype and variant CD33 (amino acids D18 to H259; Uniprot P20138-1) were expressed with an N-terminal His6 tag in CHOEBNALT85 cell line at 50-100 ml. Capture from the cell culture supernatants was done using 5 mL HisTrap Excel columns, in some cases followed by gel filtration using Superdex 75 and subsequent sterile filtration. The extracellular domains of CD33 R119A and of the double variant F21S_W22R were purified using IMAC chromatography before buffer exchange to PBS pH 7.4. The recombinant purified CD33 extracellular domains were then tested in various biophysical assays to assess protein integrity and stability versus the wildtype protein.

Elected variants with associated point mutations should not affect, or only marginally affect, the structural integrity of the protein and therefore retain the biophysical properties of the surface antigen. Comparison of each parameter to wildtype CD33 makes sure that properties close to the non-mutated CD33 are retained.

To test CD33 variants, CD33 wildtype and variants were diluted to 1 mg/mL, combined with Sypro Orange (Sigma-Adrich, S5692) and exposed to a temperature gradient from 25° C. to 90° C. utilizing a RT-PCR machine (Biorad, C1000 Thermal Cycler). The fluorescence is monitored as a function of the temperature. Initial fluorescence at 25° c. comes from the raw data and the first derivative curve provides the melting temperature in degrees Celsius.

Monomeric content of all variants was assessed after capture purification by HPLC Size Exclusion Chromatography (Waters BioSuite 250 4 um UHR SEC 4.6×300 mm or AdvanceBio SEC 300A 2.7 um 4.6×300 mm). Protein peaks were monitored at 220 and 280 nm and the relative peak areas were measured in %.

Results are shown in Table 18:

| | % of | % of | RFU at | Melting |
|---|---|---|---|---|
| | monomer | monomer | 25° C. at | temperature |
| Variants | post capture | post SEC | 1 mg · ml | (° c.) |
| WT | 100 | 100 | 1295.15 | 58.0 |
| N20G | 64.3 | 99.41 | 2282.17 | 59.0 |
| N20S | 69 | 100 | 2840.02 | 58.5 |
| N20D | 78 | 100 | 2502.81 | 58.5 |
| N20E | 9 | 100 | 3879.46 | 59.0 |
| N20K | 94 | 100 | 1289.30 | 58.5 |
| N20V | 92 | 100 | 2432.14 | 58.0 |
| N20R | 69 | 100 | 2573 | 58.8 |
| N20H | 91 | 100 | 2579 | 58.0 |
| F21V | 89 | 100 | 1945.39 | 60.0 |
| F21L | 69.4 | 100 | 1091.30 | 57.5 |
| F21I | 94 | 100 | 655.41 | 60.3 |
| F21S | 96 | 100 | 248.59 | 59.0 |
| F21M | 94 | 100 | 2599.08 | 57.25 |
| F21N | 95 | 100 | 2567.22 | 56.0 |
| F21Q | 94 | 100 | 2574.63 | 56.5 |
| F21H | 96 | 100 | 2568.02 | 56.5 |
| W22S | 91 | 100 | 2695.88 | 56.8 |
| W22T | 94 | 99.41 | 2800.89 | 57.0 |
| W22E | 96 | 100 | 793.18 | 55.0 |
| W22R | 92 | 100 | 926.82 | 57.0 |
| W22D | 95 | 100 | 2568.45 | 53.5 |
| W22K | 95 | 100 | 2578.17 | 56.3 |
| W22H | 95 | 100 | 2554.64 | 55.5 |
| Q24R | 92 | 100 | 397.20 | 58.0 |
| F43S | 66 | 95 | 14213.96 | 44.5 |
| F44P | 82 | 100 | 8606.80 | 44.5 |
| F44S | 95 | 100 | 774.32 | 58.0 |
| H45Y | 97 | 100 | 2250.91 | 63.5 |
| P48F | 96 | 100 | 2022.42 | 57.5 |
| P48D | 97 | 100 | 1300.10 | 57.5 |
| P48S | 96 | 100 | 1388.18 | 58.0 |
| Y50S | 97 | 100 | 979.46 | 58.0 |
| Y50D | 97 | 100 | 908.99 | 58.0 |
| Y50R | 96 | 100 | 790.52 | 58.5 |
| Y50L | 96 | 100 | 1039.20 | 57.0 |
| Y50E | 95 | 100 | 2555.15 | 59.5 |
| Y50K | 96 | 100 | 2574.75 | 58.8 |
| Y50H | 97 | 100 | 2590.68 | 58.5 |
| Y50H_Y49H | 93 | 100 | 604.32 | 59.0 |
| N100Q | 71 | 100 | 2583.17 | 55.5 |
| R119A | 97 | Not tested | 2848 | 58.0 |
| F21S_W22R | 95 | Not tested | 2847 | 56.3 |

All CD33 variants except for N20S, N20D, N20E, N20G, F21L and F43S show low aggregation propensity similar to wildtype CD33 with a monomer fraction of >90% and are thus preferred variants regarding aggregation.

All CD33 variants except for F43S and F44P show high thermal stability as measured by their Tm (° C.), exhibiting a protein stability similar to wildtype CD33 and are thus preferred variants regarding thermal stability.

### Example 22: Engraftment and Differentiation of Edited Cells in Mice

Human HSPCs were edited as described in Example 11 using sgRNA-E. Cells were frozen in CryoStor CS10 medium (Stem Cell Technologies #07930) two days after electroporation. After thawing and washing, ^{sgRNA-E}HSPCs (each 0.78 million) and ^{NTC}HSPCs (each 0.72 million cells) or saline were immediately injected into the tail vein of female 4-week old NBSGW mice (Jackson Laboratory; stock #026622). After 13 weeks, the peripheral blood and the bone marrow of the mice were analyzed to see if the edited HSPCs differentiate or engraft differently than unedited HSPCs. To do so, after euthanasia of the mice, 0.2 ml of blood and both posterior legs were collected from each mouse. Cell suspensions were generated, red blood cells were lysed using ACK lysis buffer and then the cell suspension was filtered. All cells were stained with a viability dye (Zombie UV Fixable Viabilty Kit 1:1000, Biolegend #423108).

Bone marrow cells were then stained with CD33 FITC (1:50, Biolegend #303304), CD34 PerCP-Cy5.5 (1:10, BD Biosciences #347222), CD14 PE (1:50, Biolegend #982508), CD10 PECF594 (1:50, BD Biosciences #562396), CD90 PE-Cy7 (1:50, BD Biosciences #561558), CD33 APC (Refmab #3), CD38 AF700 (Beckman Coulter #B23489), CD45 RA APC/Fire (1:50, Biolegend #304152), CD3 BV421 (1:50, Biolegend #300434), CD45 V500 (1:50, BD Biosciences #560777), mCD45 BV605 (1:200, BD Biolegend #103155), CD123 BV650 (1:50, Biolegend #306020) and CD117 BV711 (1:50, Biolegend #313230), FcR blocking agent human (1:200, Miltenyi #130-059-901) was added.

Peripheral blood cells were then stained with CD33 FITC (1:50, Biolegend #303304), CD34 PerCP-Cy5.5 (1:100, BD Biosciences #347222), CD335 PE (1:50, BD Biosciences #557991), CD11c PECF594 (1:50, BD Biosciences #562393), CD13 PE-Cy7 (1:50, Biolegend #301712), CD33 APC (Refmab #3), mCD45 AF700 (Biolegend #103128), CD14 APC-Cy7 (1:50, Biolegend #301820), CD3 BV421 (1:50, Biolegend #300434), CD45 V500 (1:50, BD Biosciences #560777), CD123 BV650 (1:50, Biolegend #306020), CD117 BV711 (1:50, Biolegend #313230) and CD19 BV786 (1:50, BD Biosciences #363028), FcR blocking agent human (1:200, Miltenyi #130-059-901) was added. Data was recorded using BD SSRFortessa instruments with the BD FACSDiva software and analyzed with FlowJo software.

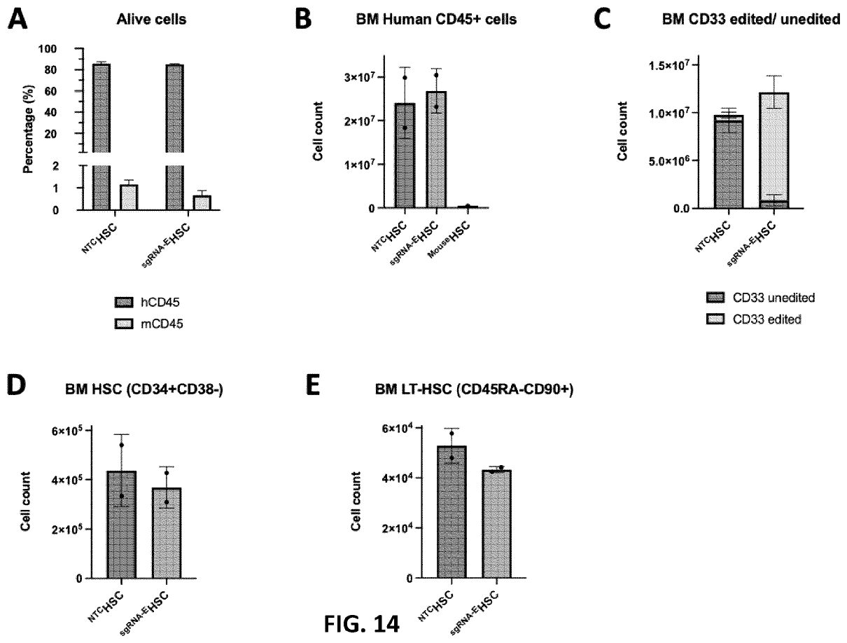

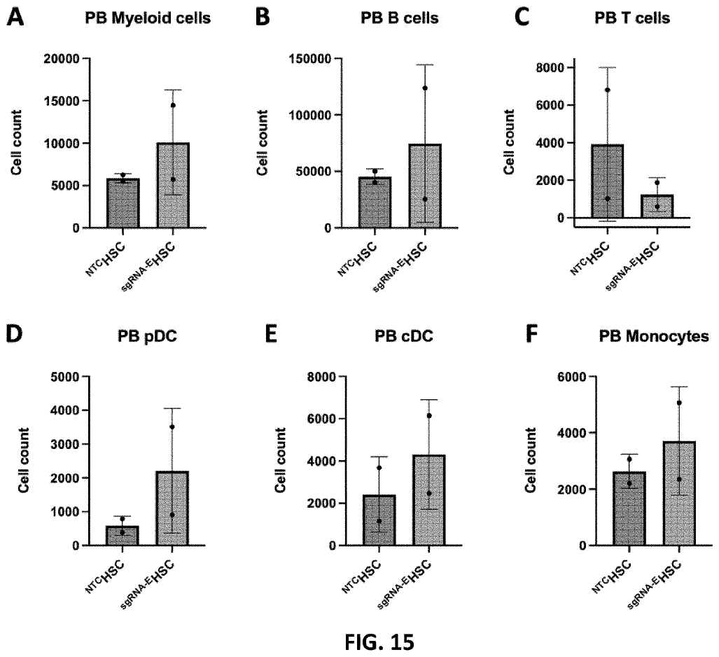

Results can be seen in FIGS. 14 and 15. ^{sgRNA-E}HSPCs and ^{NTC}HSPCs nearly completely replace the mouse hematopoietic system. Similar frequencies of HSC (CD34+, CD38−) and LT-HSC (CD34+, CD38−, CD45 RA−, CD90+) are seen in both conditions (FIG. 14 D, E). There is no selective advantage for unedited HSPCs as frequency of edited cells within ^{sgRNA-E}HSPCs after 13 weeks (FIG. 14C) is comparable to distribution before injection. Differentiation of ^{sgRNA-E}HSPCs and ^{NTC}HSPCs is indistinguishable (FIG. 15).

In conclusion, it was observed that edited and unedited HSCP show indistinguishable differentiation profiles and comparable amounts of engraftment.

### Example 23: In Vivo Efficacy of an Anti-CD33 ADC in Mice Transplanted with AML Cell Lines

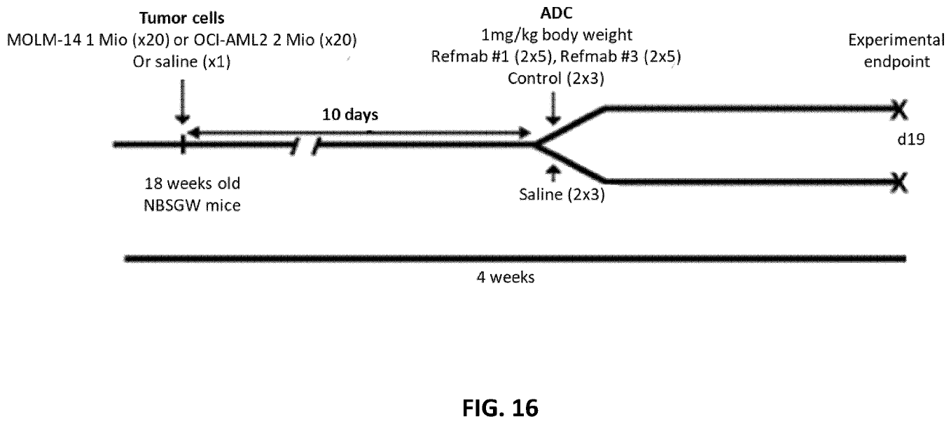

In this experiment, AML cell lines mCherry-luc labelled MOLM-14 (1 million cells; RRID:CVCL_7916) and mCherry-luc labelled OCI-AML2 cells (2 million cells; RRID:CVCL_1619) were transplanted into the tail vein of 18 week old NBSGW mice (Jackson Laboratory; stock #026622). After 10 days mice were treated with saline, or ADC (Mylotarg, Refmab #3-ADC or a control antibody against chicken lysozyme at 1 mg/kg body weight. An experimental outline is shown in FIG. 16.

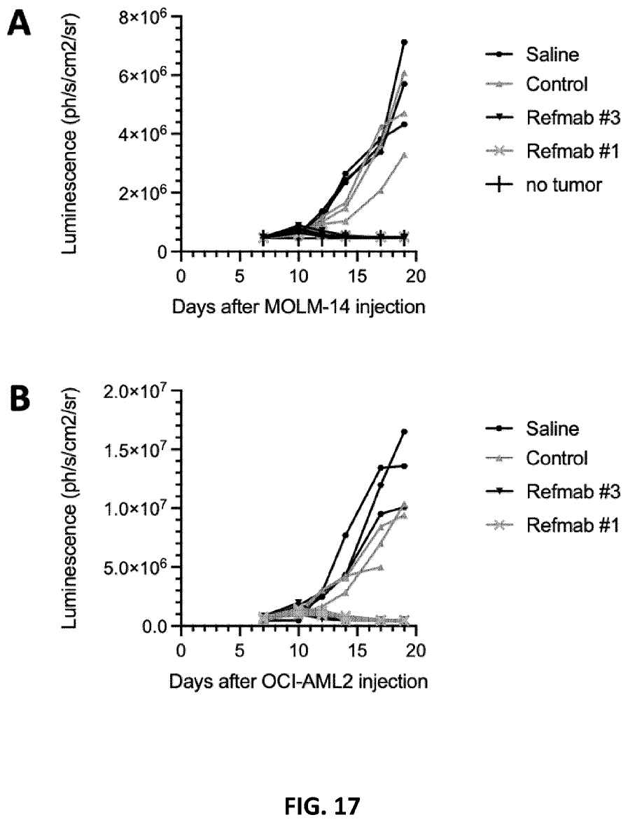

Growth of the tumor cell lines was measured by repetitive luminescence measurement after intraperitoneal injection of 100 μl D-Luciferin (BioSynth #L-8220) using the Newton7.0 imaging (Vilber). Results are shown in FIG. 17. It can be clearly seen that tumor growth continues in mice treated with saline or the control ADC, but tumor growth is effectively eliminated in mice treated with antibodies Refmab #1 or Refmab #3. The same observation could be made for both AML cell lines tested.

Tumor cells, as measured by positive staining for hCD45 cells were quantified in blood and bone marrow at the experimental endpoint at day 19. After euthanasia of the mice, 0.2 ml of blood and both posterior legs were collected from each mouse. Cell suspensions were generated, red blood cells were lysed using ACK lysis buffer and then the cell suspension was filtered. All cells were stained with a viability dye (Zombie UV Fixable Viabilty Kit 1:1000, Biolegend #423108).

Bone marrow cells were stained with the following antibodies: CD33 FITC (1:50, Biolegend #303304), CD34 PerCP-Cy5.5 (1:10, BD Biosciences #347222), CD45 PE (1:200, Biolegend #393412), CD90 PE-Cy7 (1:50, BD Biosciences #561558), CD33 APC (Refmab #3), CD38 AF700 (Beckman Coulter #B23489), CD45 RA APC/Fire (1:50, Biolegend #304152), CD3 BV421 (1:50, Biolegend #300434), CD45 V500 (1:50, BD Biosciences #560777), mCD45 BV605 (1:200, BD Biolegend #103155), CD123 BV650 (1:50, Biolegend #306020) and CD117 BV711 (1:50, Biolegend #313230), FcR blocking agent human (1:200, Miltenyi #130-059-901) was added.

Peripheral blood cells were then stained with CD33 FITC (1:50, Biolegend #303304), CD34 PerCP-Cy5.5 (1:100, BD Biosciences #347222), CD45 PE (1:200, Biolegend #393412), CD13 PE-Cy7 (1:50, Biolegend #301712), CD33 APC (Refmab #3), mCD45 AF700 (Biolegend #103128), CD14 APC-Cy7 (1:50, Biolegend #301820), CD3 BV421 (1:50, Biolegend #300434), CD45 V500 (1:50, BD Biosciences #560777), CD123 BV650 (1:50, Biolegend #306020), CD117 BV711 (1:50, Biolegend #313230) and CD19 BV786 (1:50, BD Biosciences #363028), FcR blocking agent human (1:200, Miltenyi #130-059-901) was added.

Data acquisition and analysis was performed as described for example 22.

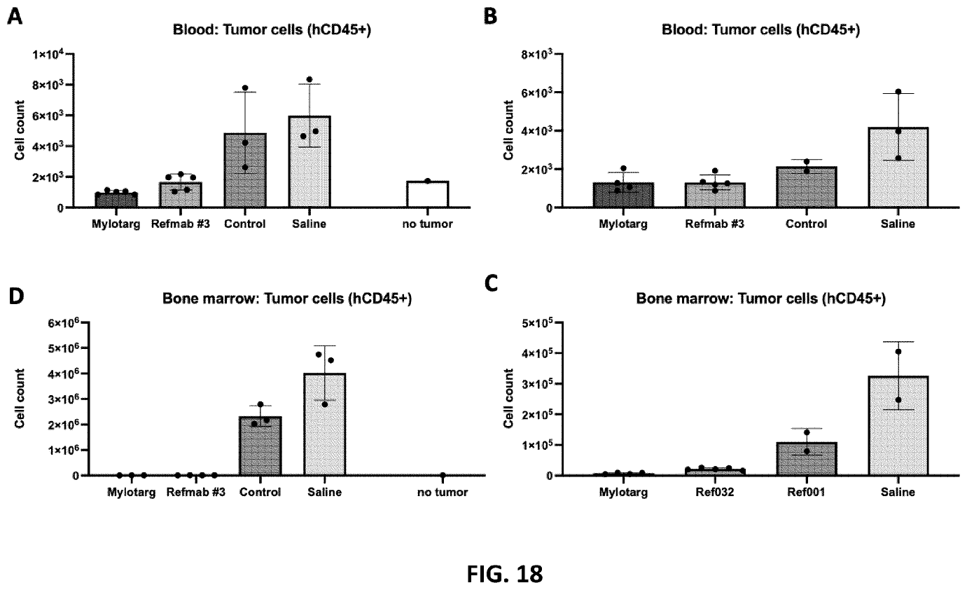

Results are shown in FIG. 18. Treatment with Refmab #1 and Refmab #3 led to cell numbers comparable to mice into which no tumors had been transplanted. ADC's of Refmab #1 and Refmab #3 ADC effectively eliminated AML-cell lines MOLM-14 and OCI-AML2 in vivo.

### Example 24: In Vitro Killing Assay

In order to show selective killing of ^{sgRNA-E}HSPCs, killing assays are performed starting 5 days after electroporation of the HSCPs to allow for completed editing and expression of the CD33 variant on the edited cells. MOLM-14, ^{NTC}HSPCs and sgRNA-EHSPCs are incubated with increasing concentrations of Refmab #3 ADC.

After 7 days, cells are harvested and stained with a viability dye as well as Refmab #3 and the control antibody CD33 HIM3-4. MOLM-14 and ^{NTC}HSPCs are killed, whereas sgRNA-EHSPCs are shielded from the ADC toxicity due to the lost binding of Refmab #3.

### Example 25: In Vivo Experiments

To show shielding against Refmab #3 targeting therapies of ^{sgRNA-E}HSPCs in contrast to ^{NTC}HSPCs, in vivo experiments with humanized NBSGW mice are performed. Stable engraftment is confirmed by flow cytometry of the peripheral blood. Animals are then treated with either Refmab #3 ADC or CAR-T. Three weeks post-treatment, mice will be sacrificed and bone marrow, spleen and peripheral blood will be assessed for engraftment and differentiation.

50% of the bone marrow of the previously described experiment are then used for secondary transplants in NSG-SGM3 mice to show successful editing of LT-HSC, which are able to engraft in a secondary host.

The model will also be used prove persistent human hematopoiesis after treatment with Refmab #3 in humanized NBSGW mice challenged by injection of tumor cells.

1: A mammalian cell or a population of cells expressing a first isoform of CD33 for use in a medical treatment in a patient in need thereof, said patient having cells expressing a second isoform of CD33,
wherein said cell expressing said first isoform comprises a polymorphism or genetically engineered allele that is not present in the genome of the patient having cells expressing said second isoform of CD33, wherein said first and second isoform are substantially functionally identical and/or are expressed at the same level or substantially the same level, and
wherein said polymorphic or genetically engineered allele is characterized by at least one substitution of an amino acid at position N20, F21, W22, Q24, F43, F44, H45, P48, Y49 and/or Y50 of SEQ ID NO: 1.

2: The mammalian cell or population of cells for use according to claim 1, wherein said polymorphic or genetically engineered allele is characterized by at least one substitution of an amino acid at position N20, F21, W22 and/or Y50 of SEQ ID NO: 1.

3: The mammalian cell or population of cells for use according to claim 1, wherein said function is the binding to sialic acids, the stimulation by sialic acids, phosphorylation upon binding to sialic acids, the capability to act as a docking site for Src homology 2 (SH2) domain-containing proteins and/or are the capability to trigger the inhibition of phagocytosis.

4: The mammalian cell or population of cells for use according to claim 3, wherein said residue N20 is substituted with G, S, D, E, K, V, R or H, and/or residue F21 is substituted with V, L, I, S, M, N Q or H, and/or residue W22 is substituted with S, T, E, R, D, K or H, and/or Q24 is substituted with R, and/or F43 is substituted with S, and/or F44 is substituted with P or S, and/or H45 is substituted with Y, and/or residue P48 is substituted with F, D or S, and/or residue Y49 is substituted with A, and/or residue Y50 is substituted with S, L, R, D, E, A, K or H.

5: The mammalian cell or population of cells for use according to claim 1, wherein said first isoform of CD33 is obtained by in vivo or ex vivo modifying the nucleic acid sequence encoding said first isoform of CD33 by gene editing, preferably by introducing into a cell a gene editing enzyme capable of inducing site-specific mutations(s) within a target sequence encoding a surface protein region involved in the binding of agent comprising at least a first antigen-binding region.

6: The mammalian cell or population of cells according to claim 1 wherein said medical treatment comprises administering a therapeutically efficient amount of said cell or population of cells expressing said first isoform of CD33 to said patient in need thereof, in combination with a therapeutically efficient amount of a depleting agent comprising at least a first antigen-binding region that binds specifically to said second isoform of CD33 to specifically deplete patient cells expressing said second isoform of CD33.

7: The mammalian cell or population of cells for use according to claim 6, wherein said first antigen-binding region of said depleting agent binds specifically to an epitope including the amino acids N20, F21 and/or W22 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9.

8: The mammalian cell or population of cells for use according to claim 6, wherein said first antigen-binding region of said depleting agent binds specifically to an epitope including the amino acids N20, F21 and/or W22 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24.

9: The mammalian cell or population of cells for use according to claim 6, wherein said first antigen-binding region of said depleting agent binds specifically to an epitope including amino acid P48 and/or Y50 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32.

10: A pharmaceutical composition comprising the mammalian cell or population of cells of claim 1 and a pharmaceutically acceptable carrier.

11: A depleting agent for use in preventing or reducing the risk of severe side effects in a patient having received a cell expressing a first isoform of CD33, wherein said patient's native cells express a second isoform of CD33, and wherein said depleting agent comprises at least a second antigen-binding region which binds specifically to said first isoform of CD33 and does not bind or binds substantially weaker to said second isoform of CD33.

12: A depleting agent for use in selectively depleting host cells in a patient in need thereof wherein said patient's host cells express a second isoform of CD33 and wherein said depleting agent comprises at least a first antigen-binding region which binds specifically to said second isoform of CD33, wherein said first antigen-binding region of said depleting agent binds specifically to an epitope including the amino acids N20, F21 and/or W22 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 4, VHCDR2 is SEQ ID NO: 5 and VHCDR3 is SEQ ID NO: 6; and
b) an antibody light chain variable domain (VL) comprising the three CDRs VLCDR1, VLCDR2 and VLCDR3 wherein VLCDR1 is SEQ ID NO: 7, VLCDR2 is SEQ ID NO: 8, VLCDR3 is SEQ ID NO: 9.

13: A depleting agent for use in selectively depleting host cells in a patient in need thereof wherein said patient's host cells express a second isoform of CD33 and wherein said depleting agent comprises at least a first antigen-binding region which binds specifically to said second isoform of CD33, wherein said first antigen-binding region of said depleting agent binds specifically to an epitope including the amino acids N20, F21, and/or W22 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 20, VHCDR2 is SEQ ID NO: 21 and VHCDR3 is SEQ ID NO: 22; and
b) an antibody light chain variable domain (VL) comprising the three CDRs VLCDR1, VLCDR2 and VLCDR3 wherein VLCDR1 is SEQ ID NO: 23, VLCDR2 is SEQ ID NO: 8, VLCDR3 is SEQ ID NO: 24.

14: A depleting agent for use in selectively depleting host cells in a patient in need thereof wherein said patient's host cells express a second isoform of CD33 and wherein said depleting agent comprises at least a first antigen-binding region which binds specifically to said second isoform of CD33, wherein said first antigen-binding region of said depleting agent binds specifically to an epitope including amino acid Y50 of SEQ ID NO: 1, and wherein said first antigen-binding region comprises an antigen binding region which has the same epitope specificity as an antigen binding region comprising:
a) an antibody heavy chain variable domain (VH) comprising the three CDRs VHCDR1, VHCDR2 and VHCDR3 wherein VHCDR1 is SEQ ID NO: 27, VHCDR2 is SEQ ID NO: 28 and VHCDR3 is SEQ ID NO: 29; and
b) an antibody light chain variable domain (VL) comprising the three CDRs VLCDR1, VLCDR2 and VLCDR3 wherein VLCDR1 is SEQ ID NO: 30, VLCDR2 is SEQ ID NO: 31, VLCDR3 is SEQ ID NO: 32.

15: The mammalian cell or population of cells for use according to claim 1 comprising hematopoietic stem cells.

16: The mammalian cell or population of cells for use according to claim 6 which restores normal haematopoiesis after immunotherapy in the treatment of hematopoietic disease.

17: The mammalian cell or population of cells for use according to claim 16 wherein the hematopoietic disease is acute myeloid leukemia (AML), myelodysplastic syndrome (MDS), chronic myeloid leukemia (CML), chronic myelomonocytic leukemia (CMML), polycythemia vera (PV), essential thrombocythemia (ET), primary myelofibrosis (PMF), blastic plasmacytoid dendritic cell neoplasm (BPDCN) or another myeloproliferative neoplasm.

18: The mammalian cell or population of cells for use according to claim 6 wherein said depleting agent is an antibody, antibody-drug conjugate or an immune cell.

19: The mammalian cell or population of cells for use according to claim 6 wherein said depleting agent a T-cell bearing a chimeric antigen receptor (CAR) comprising a first antigen-binding region which binds specifically to said second isoform and does not bind or binds substantially weaker to said first isoform.

20: The pharmaceutical composition of claim 10 further comprising a depleting agent comprising at least a first antigen-binding region that binds specifically to said second isoform of CD33 to specifically deplete patient cells expressing said second isoform of CD33.
